(19) United States
(12) Patent Application Publication
(10) Pub. No.: US 2026/0167636 A1
(43) Pub. Date: Jun. 18, 2026

(54) NOVEL COMPOUNDS FOR THE DIAGNOSIS OF TDP-43 PROTEINOPATHIES

(71) Applicant: AC IMMUNE SA, Lausanne (CH)

(72) Inventor: Nicolas Dreyfus, Lausanne (CH)

(21) Appl. No.: 19/126,934

(22) PCT Filed: Oct. 31, 2023
(86) PCT No.: PCT/EP2023/080426

§ 371 Date: May 2, 2025

(30) Foreign Application Priority Data
Nov. 2, 2022 (EP) 22205175.7

(51) Int. Cl. C07D 417/14; A61K 51/04; C07B 59/00; G01N 33/68

(52) U.S. Cl. CPC C07D 417/14; A61K 51/0455; A61K 51/0459; C07B 59/002; G01N 33/6896; A61K 2123/00; G01N 2800/2814

(57) ABSTRACT

The present invention relates to compounds which are suitable for imaging TDP-43 (Transactive response (TAR) DNA binding protein 43 kDa) aggregates. The compounds can be used, for example, for diagnosing a disease, disorder or abnormality associated with TDP-43 aggregates or a TDP-43 proteinopathy, such as amyotrophic lateral sclerosis (ALS), Alzheimer's disease (AD), Frontotemporal dementia (FTD) and limbic-predominant age-related TDP-43 encephalopathy (LATE).

### FIELD OF THE INVENTION

The present invention relates to compounds which are suitable for imaging TDP-43 (Transactive response (TAR) DNA binding protein 43 kDa) aggregates. Said compounds can be used, for example, for diagnosing a disease, disorder or abnormality associated with TDP-43 aggregates or a TDP-43 proteinopathy, such as amyotrophic lateral sclerosis (ALS), Alzheimer's disease (AD), Frontotemporal dementia (FTD) and limbic-predominant age-related TDP-43 encephalopathy (LATE). The present invention also relates to processes for the preparation of said compounds, diagnostic compositions comprising said compounds, methods of using said compounds, kits comprising said compounds and the uses thereof.

### BACKGROUND OF THE INVENTION

Age-associated brain disorders characterized by pathological aggregation of proteins in the central nervous system (CNS) (proteinopathies) and peripheral organs represent one of the leading causes of disability and mortality in the world. The best characterized protein that forms extracellular aggregates is amyloid beta (Abeta) in Alzheimer's disease (AD) and Abeta-related disorders. Other disease-associated, aggregation-prone proteins leading to neurodegeneration include but are not limited to, Tau, alpha-synuclein (a-syn), huntingtin, fused in sarcoma (FUS), dipeptide repeat proteins (DPRs) produced by unconventional translation of the C9orf72 repeat expansion, superoxide dismutase 1 (SOD1), and TDP-43. Diseases involving TDP-43 aggregates are generally referred to as TDP-43 proteinopathies and include, but are not limited to, amyotrophic lateral sclerosis (ALS) and frontotemporal lobar degeneration (FTLD), including frontotemporal lobar dementia with TDP-43 pathology (FTLD-TDP, Frontotemporal lobar degeneration with TDP-43 inclusions) and limbic-predominant age-related TDP-43 encephalopathy (LATE).

### TDP-43 Introduction

Transactive response (TAR) DNA binding protein 43 kDa (TDP-43) is a 414-amino acid protein encoded by the TARDBP gene on chromosome 1p36.2 (ALS10). TARDBP is comprised of six exons (exon 1 is non-coding; exons 2-6 are protein-coding). TDP-43 belongs to the family of heterogeneous ribonucleoprotein (hnRNP) RNA binding proteins (Wang et al., Trends in Molecular Medicine, Vol. 14, No. 11, 2008, 479-485; Lagier-Tourenne et al., Human Molecular Genetics, 2010, Vol. 19, Review Issue 1 R46-R64). TDP-43 contains five functional domains (FIG. 1 in Warraich et al., The International Journal of Biochemistry & Cell Biology, 42 (2010) 1606-1609): two RNA recognition motifs (RRM1 and RRM2), which have two highly conserved hexameric ribonucleoprotein 2 (RNP2) and octameric ribonucleioprotein 1 (RNP1) regions, a nuclear export signal (NES) and a nuclear localization signal (NLS) enabling it to shuttle between the nucleus and the cytoplasm transporting bound mRNA, and a glycine rich domain at the C-terminal, which mediates protein-protein interactions. TDP-43 is involved in multiple aspects of RNA processing, including transcription, splicing, transport, and stabilization (Buratti and Baralle, FEBS Journal, 277 (2010) 2268-2281). It is a highly conserved, ubiquitously expressed protein with a tightly autoregulated expression level that shuttles continuously between the nucleus and cytoplasm but is normally localized predominantly to the nucleus. In 2006, TDP-43 was identified as the protein that accumulates in most cases of frontotemporal lobar degeneration (FTLD) with tau-negative, ubiquitin-positive inclusions (then referred to as FTLD-TDP), and in most cases of amyotrophic lateral sclerosis (ALS) (Arai et al., Biochemical and Biophysical Research Communications, 351 (2006) 602-611; Neumann et al., Science, 314, (2006), 130-133).

Thirty-eight negative-dominant mutations in TDP-43 have been identified in sporadic and familial ALS patients as well as in patients with inherited FTD (K263E, N267S), mainly located in the glycine-rich domain (FIG. 1; Lagier-Tourenne and Cleveland, Cell, 136, 2009, 1001-1004). TDP-43 is inherently aggregation-prone, as shown by sedimentation assays, and this propensity is increased by some ALS-associated TARDBP mutations (Ticozzi et al., CNS Neurol. Disord. Drug Targets, 2010, 9(3), 285-296).

### TDP-43 in Neurodegeneration

TDP-43 aggregates have been identified in a growing list of pathological conditions (Lagier-Tourenne et al., Human Molecular Genetics, 2010, Vol. 19, Review Issue 1 R46-R64), including but not limited to: frontotemporal dementia (sporadic or familial with or without motor-neuron disease (MND), with progranulin (GRN) mutation, with TARDBP mutation, with valosine-containing protein (VCP) mutation, linked to chromosome 9p, corticobasal degeneration, frontotemporal lobar degeneration with ubiquitin-positive inclusions, argyrophilic grain disease, Pick's disease and the like), amyotrophic lateral sclerosis (sporadic ALS, with TARDBP mutation, with ANG mutation), Alzheimer's disease (sporadic and familial), Down syndrome, familial British dementia, polyglutamine diseases (Huntington's disease and SCA3), hippocampal sclerosis dementia and myophaties (sporadic inclusion body myositis, inclusion body myopathy with VCP mutation, oculo-pharyngeal muscular dystrophy with rimmed vacuoles, myofibrillar myopathies with MYOT or DES mutation).

Aggregated TDP-43 from patient brains shows a number of abnormal modifications, including hyperphosphorylation, ubiquitination, acetylation and C-terminal fragments through proteolytic cleavage (Arai et al., Biochemical and Biophysical Research Communications, 351 (2006) 602-611; Neumann et al., Science, 314, (2006), 130-133; Neumann et al., Acta Neuropathol., (2009) 117: 137-149; Hasegawa et al., Annals of Neurology, 2008, Vol 64 No 1, 60-70; Cohen et al., Nat Commun.; 2015, 6: 5845). Another characteristic feature of TDP-43 pathology is redistribution and accumulation of TDP-43 from nucleus to cytoplasm. The hallmark lesions of FTLD-TDP are neuronal and glial cytoplasmic inclusions (neuronal cytoplasmic inclusions (NCI) and glial cytoplasmic inclusions (GCI), respectively) and dystrophic neurites (DN) that are immunoreactive for TDP-43, as well as ubiquitin and p62, but negative for other neurodegenerative disease-related proteins. Differences in inclusion morphology and tissue distribution thereof are associated with specific mutations and/or clinical representations. Four types of TDP-43 pathology have been described so far by histological methods (Mackenzie and Neumann, J. Neurochem., (2016), 138 (Suppl. 1), 54-70). FTLD-TDP type A cases are characterized by abundant short DN and compact oval or crescentic NCI, predominantly in layer II of the neocortex (FIG. 2f in Mackenzie et al., J. Neurochem., 2016, 138 (Suppl. 1), 54-70). Cases with this pathology usually present clinically with either behavioral-variant frontotemporal dementia (bvFTD) or nonfluent/agrammatic variants of Primary Progressive Aphasia (nfvPPA) and are associated with progranulin (GRN) mutations. Type B cases show moderate numbers of compact or granular NCI in both superficial and deep cortical layers with relatively few DN and Nil (FIG. 2g in Mackenzie et al., J. Neurochem., 2016, 138 (Suppl. 1), 54-70). Most cases with coappearence of FTD and ALS symptoms are found to have FTLD-TDP type B pathology. Type C cases have an abundance of long tortuous neurites, predominantly in the superficial cortical laminae, with few or no NCI (FIG. 2j in Mackenzie et al., J. Neurochem., 2016, 138 (Suppl. 1), 54-70). This pathology is particularly found in cases presenting with svPPA (semantic variant of primary progressive aphasia). FTLD-TDP type D displays with abundant lentiform neuronal intranuclear inclusions (Nil) and short DN in the neocortex with only rare NCI (FIG. 2k in Mackenzie et al., J. Neurochem., 2016, 138 (Suppl. 1), 54-70). This pattern of pathology is only found in cases with VCP in association with inclusion body myositis.

### TDP-43 in FTD

Frontotemporal dementia (FTD) is a clinical term that covers a wide spectrum of disorders based on the degeneration of frontal and temporal lobes—a pathological feature termed frontotemporal lobar degeneration (FTLD). FTD is the second most abundant cause of early degenerative dementias in the age group below 65 years (Le Ber, Revue Neurologique, 169 (2013), 811-819). FTD is presented by several syndromes including bvFTD which is characterized by changes in personality and behavior; semantic dementia (SD) and progressive nonfluent aphasia (PNFA) characterized by changes in the language function; corticobasal syndrome (CBS), progressive supranuclear palsy syndrome and motor neuron disease (FTD-MND) characterized by movement disfunction. Diagnosis of these syndromes is complicated and final conclusion can only be achieved through postmortem tissue analysis based on immunohistochemistry to detect aggregated protein and description of the affected brain regions. In terms of pathological, proteinaceaous inclusions, about 45% of cases show pathological accumulation of misfolded Tau, 45% of cases have pathological TDP-43 and a smaller subgroup has aggregates of FUS and other proteins. FTLD-TDP is a pathology term describing FTD cases with TDP-43 pathology found predominantly as cytoplasmic or neuritic protein aggregates in neurons and glia containing misfolded, insoluble, phosphorylated and truncated TDP-43.

### TDP-43 in ALS

Amyotrophic lateral sclerosis (ALS) is an adult-onset neurodegenerative disorder characterized by the premature loss of upper and lower motor neurons. The progression of ALS is marked by fatal paralysis and respiratory failure with a disease course from diagnosis to death of 1 to 5 years. In most cases of sporadic ALS, neuropathology is characterized by abnormal cytoplasmic accumulations of TDP-43 in neurons and glia of the primary motor cortex, brainstem motor nuclei, spinal cord and the associated white matter tracts. ALS with dementia involves accumulation of TDP-43 in extramotor neocortex and hippocampus. The role of phosphorylation of TDP-43 in ALS patients has been explored with the help of phospho-specific antibodies that strongly bind to nuclear and cytoplasmic TDP-43 inclusions. Amino acids S379, S403, S404, 5409, and S410 have been identified as the major sites of phosphorylation of TDP-43 (Hasegawa et al., Ann Neurol., 2008; 64: 60-70; Neumann et al., Acta Neuropathol., 2009, 117: 137-149).

### TDP-43 in LATE

Limbic-predominant age-related TDP-43 encephalopathy (LATE) neuropathological change (LATE-NC) is defined by a stereotypical TDP-43 proteinopathy in older adults, with or without coexisting hippocampal sclerosis pathology. LATE-NC is a common TDP-43 proteinopathy, associated with an amnestic dementia syndrome that mimicked Alzheimer's-type dementia in retrospective autopsy studies. LATE is distinguished from frontotemporal lobar degeneration with TDP-43 pathology based on its epidemiology (LATE generally affects older subjects), and relatively restricted neuroanatomical distribution of TDP-43 proteinopathy. There is no molecule-specific biomarker for LATE. A discovery of a TDP-43 PET tracer may enable accurate, potentially earlier diagnosis as well as monitoring of disease progression to facilitate longitudinal drug efficacy measurements in patients during clinical trials (including as a potential exclusion criterion for Alzheimer's disease clinical trials) and longitudinal studies of the clinical and pathological progression of LATE (Nelson et al., Brain, 2019, Vol. 142; issue 6, 1503-1527).

### TDP-43 in AD and Other Diseases

TDP-43 pathology occurs in up to 57% of brains of patients with Alzheimer's disease (Josephs K A et al., Acta Neuropathol., 2014; 127(6): 811-824; Josephs K A et al., Acta Neuropathol., 2014; 127(3): 441-450; McAleese et al., Brain Pathol., 2017 July; 27(4): 472-479). TDP-43 aggregation is associated with cognitive decline, memory loss and medial temporal atrophy in AD. TDP-43 positive patients are 10-fold more likely to be cognitively impaired at death compared to TDP-43 negative subjects. It appears that TDP-43 represents a secondary or independent pathology that shares overlapping features with AD by targeting the medial temporal lobe. Pathologic TDP-43 follows a stereotypical pattern of deposition that was captured by the TDP-43 in AD (TAD) staging scheme: TDP-43 first deposits in the amygdala (stage 1) followed by hippocampus, limbic, temporal, and finally frontostriatum (stage V) (Josephs K A et al., Acta Neuropathol., 2014; 127(6): 811-824; Josephs K A et al., Acta Neuropathol., 2014; 127(3): 441-450).

### Diagnostics in FTD and ALS

The diagnosis of FTD based on clinical manifestations is insufficient since the clinical representation can overlap with other diseases, in particular, in the earlier stages. Therefore, the development of sensitive and specific biomarkers allowing the differentiation between types of pathology within the FTD spectrum is an urgent task. Such tools will allow better detecting and understanding the specific type of pathology causing neurodegeneration. Eventually this will lead to the development of diagnostic biomarkers enabling more efficient and precise patient selection for longitudinal monitoring in clinical studies, supporting the development of novel therapeutics for ALS and FTD.

A number of approaches aim at the development of biochemical biomarkers to distinguish different types of FTD pathology. Some studies showed that TDP-43 concentration is increased in cerebrospinal fluid (CSF) of clinically defined FTD or FTD-MND populations, although there is a significant overlap with control or AD subjects and it remains unclear if such an approach will prove clinically useful (Foulds et al., Acta Neuropathol., 2008, 116:141-146; Steinacker et al., Arch. Neurol., 2008; 65(11): 1481-1487). Levels of total Tau or Thr181-phosphorylated Tau do not discriminate FTLD-Tau from control. A possible diagnostic tool for the differentiation of FTLD-Tau and FTLD-TDP is the reduced CSF p-Tau181 to Tau ratio below a value of 0.37 (Hu et al., Neurology., 2013; 81(22): 1945-1952). Another study showed that CSF phosphorylated Tau levels are positively associated with cerebral Tau burden in FTD and might help to distinguish TDP-43 proteinopathy from tauopathy (Irwin et al., Ann. Neurol., 2017 August; 82(2):247-258).

In parallel to biochemical biomarkers, the development of imaging biomarkers will enable early and specific detection of the pathology in FTD, ALS and other neurodegenerative disorders. The ability to image TDP-43 deposition in the brain will be a substantial achievement for diagnosis and drug development for FTD, ALS and other neurodegenerative disorders. Progressive TDP-43 accumulation in the CNS is associated with disease progression and represents an obvious target for development of novel therapeutics and diagnostic tools to study pharmacodynamics and disease progression. Given the relative novelty of TDP-43 as a target, the development of a PET-tracer targeting this protein is at its beginning. However, most of the compounds reported so far are not specific for TDP-43 and no direct binding to the target was demonstrated for any of these compounds.

A number of challenges are associated with the development of a TDP-43-specific PET-tracer including low abundance and heterogenic distribution of the target in the patient's brain as well as the lack of reference compounds. In order to reduce background signal interference resulting from non-specific, off-target binding and to reduce dosing requirements, TDP-43 imaging compounds should bind with high affinity and selectivity to the target. For imaging of TDP-43 aggregates associated with neurological disorders such as FTD and ALS, imaging compounds need to penetrate the blood brain barrier and pass into the relevant regions of the brain. For targeting intracellular amyloid-like inclusions such as TDP-43 aggregates, cell permeability is a further requirement of imaging compounds. A further prerequisite in order to avoid accumulation of the compound in the tissue, which may result in increased risk of unwanted side effects, is a fast compound wash-out from the brain (or other target organ).

It was an object of the present invention to provide compounds which are able to bind to TDP-43 aggregates. In particular, the compounds of the present invention should be useful for identification and differentiation of patients and patient groups with TDP-43 proteinopathies (such as FTD, FTLD-TDP, LATE and ALS) and for differentiating TDP-43 proteinopathies from other proteinopathies.

The present inventors have surprisingly found that compounds having the formula (I) can recognize and bind to TDP-43 aggregates.

### SUMMARY OF THE INVENTION

The present invention is summarized in the appended claims. In particular, the present invention refers to a compound having the formula (I)

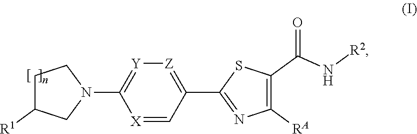

or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N;
R^{A} is H or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S,
and to compounds having the subformula (Ia)

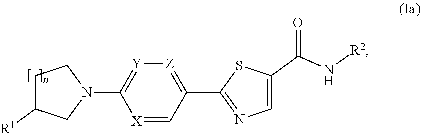

or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.
or the subformula (Ib)

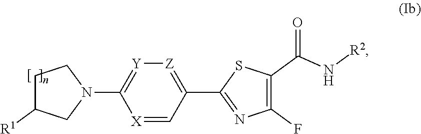

or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.

In another aspect, the present invention provides a diagnostic composition comprising a compound of formula (I), or subformulae thereof, as defined herein, and optionally at least one physiologically acceptable carrier, diluent, adjuvant and/or excipient. Said compounds can be used for imaging of TDP-43 aggregates, particularly wherein the imaging is conducted by positron emission tomography, or for diagnosing a disease, disorder or abnormality associated with TDP-43 aggregates or a TDP-43 proteinopathy, particularly wherein the diagnosis is conducted by positron emission tomography.

In another aspect, the invention provides a compound of formula (I), or subformulae thereof, which can be used in the following methods:
A method of diagnosing a disease, disorder or abnormality associated with TDP-43 aggregates or a TDP-43 proteinopathy, or a predisposition thereto, in a subject;
A method of positron emission tomography (PET) imaging of TDP-43 aggregates in a tissue of a subject;
A method for the detection and optional quantification of TDP-43 aggregates in a tissue of a subject;
A method of collecting data for the diagnosis of a disease, disorder or abnormality associated with TDP-43 aggregates or for the diagnosis of a TDP-43 proteinopathy;
A method of collecting data for determining a predisposition to a disease, disorder or abnormality associated with TDP-43 aggregates or a TDP-43 proteinopathy;
A method of collecting data for monitoring the progression of a disease, disorder or abnormality associated with TDP-43 aggregates or for monitoring the progression of a TDP-43 proteinopathy in a patient; and
A method of collecting data for predicting responsiveness of a patient suffering from a disease, disorder or abnormality associated with TDP-43 aggregates or suffering from a TDP-43 proteinopathy to a treatment with a medicament.

In another aspect, the invention provides a compound of formula (I), or subformulae thereof, that can be used as a TDP-43 aggregates' biomarker or a TDP-43 proteinopathy biomarker, as a TDP-43 proteinopathy diagnostic agent or diagnostic tool or as an in vitro analytical reference or an in vitro screening tool.

Precursors of the compound having the formula (I) or subformulae thereof, which have the formulae (II), (III) and (IV), or subformulae thereof, are also aspects of the invention.

In yet another aspect, the present invention relates to kit for preparing a radiopharmaceutical preparation, said kit comprising a precursor of a compound of formula (I), or subformulae thereof.

### BRIEF DESCRIPTION OF THE FIGURES

FIG. 1: Saturation binding curve of [³H]Compound 1 on human FTD sarkosyl insoluble brain extracts. Each point represents the average of two independent experiments ±standard error of the mean (SEM).

FIG. 2: Saturation binding curve of [³H]Compound 11 on human FTD sarkosyl insoluble brain extracts.

FIG. 3: Saturation binding curve of [³H]Compound 12 on human FTD sarkosyl insoluble brain extracts.

FIG. 4: Saturation binding curve of [³H]Compound 4 on human FTD sarkosyl insoluble brain extracts.

FIG. 5: Saturation binding curve of [³H]Compound 8 on human FTD sarkosyl insoluble brain extracts.

### DEFINITIONS

Unless defined otherwise, within the meaning of the present application the following definitions apply, and, when appropriate, a term used in the singular will also include the plural and vice versa: Compounds of the invention may have one or more optically active carbons that can exist as racemates and racemic mixtures, stereoisomers (including diastereomeric mixtures and individual diastereomers, enantiomeric mixtures and single enantiomers, mixtures of conformers and single conformers), tautomers, atropoisomers, and rotamers. All isomeric forms are included in the present invention. Compounds described in this specification containing olefinic double bonds include E and Z geometric isomers. Also included in this invention are all salt forms, such as pharmaceutically acceptable salts, polymorphs, hydrates, solvates, and mixtures thereof. Unless specified otherwise, the terms “compound of formula (X)” or “compound of the (present) invention” refer to a “compound of formula (X), or a detectably labelled compound, stereoisomer, polymorph, racemic mixture, tautomer, pharmaceutically acceptable salt, hydrate, or solvate thereof, or mixtures thereof”. Unless specified otherwise, the terms “compound of formula (X)” or “compound of the (present) invention” refer to compounds of formula (X), and subformulae thereof, and isotopically labelled compounds (including, but not limited to ¹⁸F and ³H substitutions). The terms “compound of formula (X)” or “compound of the (present) invention” refer to a compound as defined in any one of embodiments mentioned herein below.

Unless specified otherwise,

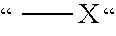

indicates that “X” is an optional substituent, i.e. “X” may be present or absent.

The term “polymorphs” refers to the various crystalline structures of the compounds of the invention. This may include, but is not limited to, crystal morphologies (and amorphous materials) and all crystal lattice forms. Salts can also be crystalline and may exist as more than one polymorph.

Solvates, hydrates as well as anhydrous forms of the salt are also encompassed by the invention. The solvent included in the solvates is not particularly limited and can be any pharmaceutically acceptable solvent. Examples include C₁₋₄ alcohols (such as methanol or ethanol).

“Pharmaceutically acceptable salts” are defined as derivatives of the compounds of the present invention wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compound of formula (I) can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, PA, 1990, p. 1445, the disclosure of which is hereby incorporated by reference. Typically, the pharmaceutically acceptable salts are salts of amine residues in the compounds of the present invention.

The “patients” or “subjects” in the present invention are typically animals, particularly mammals, more particularly humans and mice, and even more particularly humans.

A “diagnostic composition” is defined in the present invention as a composition comprising the compound of the invention in a form suitable for administration to a patient, wherein the patient is e.g. a mammal such as a human.

“TDP-43 aggregates” are TDP-43-positive multimeric rich assemblies of TDP-43. They can be found in intracellular deposits in a range of diseases termed TDP-43 proteinopathies, in particular in amyotrophic lateral sclerosis (ALS), Alzheimer's disease (AD), Frontotemporal dementia (FTD) and limbic-predominant age-related TDP-43 encephalopathy (LATE). TDP-43 aggregates can be found in the following morphologies: compact oval or crescentic neuronal cytoplasmic inclusions (NCI), lentiform neuronal intranuclear inclusions (Nil), glial cytoplasmic inclusions (GCI), dystrophic neurites (DN) and long tortous neurites. In pathological aggregates TDP-43 often displays a substantial increase in post-translational modifications such as phosphorylation, ubiquitination, acetylation, sumoylation and proteolytic cleavage to generate C-terminal fragments.

The “preclinical state” of disease is defined as the phase of disease where disease-associated changes on the molecular level are not leading to overt clinical representation in the patient.

The “clinical state” of a disease is defined as the phase of a disease where disease-associated changes on the molecular level lead to overt clinical representation in the patient.

The terms “diagnosing” or “diagnosis” generally refer to the process or act of recognizing, deciding on or concluding on a disease or condition in a patient on the basis of symptoms and signs and/or from results of a diagnostic procedure.

A “normal control value” is determined by conducting the respective method with a plurality of healthy subjects, measuring the amount of the compound bound to the TDP-43 aggregates, if any, for each healthy subject and calculating an average thereof.

A “healthy control subject (or patient)” or “healthy subject (or patient)” is a human showing no clinical evidence of neurodegenerative disease. Said human needs to meet the following criteria:
Males and females' subjects which are healthy with no clinically relevant findings upon physical examination.
No (family) history of TDP-43 proteinopathy, TDP-43 aggregates formation, or other early-onset neurological diseases associated with dementia.
No (personal) history of clinically significant neurologic (and/or psychiatric disorders for humans).
No clinical signs or symptoms of a current neurological deficit such as cognitive impairment or motor deficit.

A “preclinical control value” is determined by conducting the respective method with a plurality of subjects who are in a preclinical state, measuring the amount of the compound bound to the TDP-43 aggregates, if any, for each subject and calculating an average thereof.

A “clinical control value” is determined by conducting the respective method with a plurality of subjects who are in a clinical state, measuring the amount of the compound bound to the TDP-43 aggregates, if any, for each subject and calculating an average thereof.

The terms “predicting” or “prediction” generally refer to an advance declaration, indication or foretelling of a disease or condition in a patient not having a disease, disorder or abnormality. For example, a prediction of a disease, disorder or abnormality in a patient may indicate a probability, chance or risk that the patient will contract the disease, disorder or abnormality, for example within a certain time period or by a certain age.

Detectable labels include suitable isotopes such as radioisotopes, in particular positron emitters or gamma emitters, and include ²H, ³H, ¹⁸F, ¹²³I, ¹²⁴I, ¹²⁵I, ¹³¹I, ¹¹C, ¹³N, ¹⁵O, ^{99m}Tc and ⁷⁷Br, preferably ²H, ³H, ¹C ¹³N, ¹⁵O and ¹⁸F, more preferably ²H, ³H and ¹⁸F, even more preferably ³H and ¹⁸F, most preferably ¹⁸F.

The term “Hal”, “halogen” or “halo” means F, Cl, Br or I, particularly Br or I, more particularly Br.

The term “carbocyclic” refers to a 5- or 6-membered carbocyclic ring which is not particularly limited and includes any 5- or 6-membered, saturated or unsaturated carbocyclic ring which may be substituted or unsubstituted. Unsaturated carbocyclic rings include, but are not limited to, aromatic rings. Examples of 5- or 6-membered carbocyclic rings include, for instance, phenyl, cyclopentyl, cyclohexyl, cyclopentenyl, and cyclohexenyl. Phenyl being preferred.

The term “heterocyclic ring” refers to a stable 5- or 6-membered heterocyclic ring, is not particularly limited and includes any 5- or 6-membered, saturated or unsaturated heterocyclic ring, which may be substituted or unsubstituted. Unsaturated heterocyclic rings include, but are not limited to, aromatic rings. The heterocyclic ring contains one or more heteroatoms (for instance, one or two heteroatoms) selected from N, O and S. The heteroatom(s) is/are preferably N or S, more preferably N. Examples of 5- or 6-membered heterocyclic rings include, for instance, pyridinyl, pyrazinyl, pyrimidinyl, pyrrolyl, pyrrolidinyl, furanyl, tetrahydrofuranyl, thiophenyl, imidazolidinyl, pyrazolidinyl, imidazolyl, pyrazolyl, oxathiolidinyl, isoxthiolidinyl, oxathiolyl, isoxathiolyl, thiazolidinyl, iosthiazolidinyl, thiazolyl, and isothiazolyl, preferably pyridinyl, pyrazinyl, pyrimidinyl and isothiazolyl.

The term “leaving group” (LG) as employed herein is any leaving group and means an atom or group of atoms that can be replaced by another atom or group of atoms. Examples are given, e.g., in Synthesis (1982), p. 85-125, table 2, Carey and Sundberg, Organische Synthese, (1995), pages 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33). Preferably, the “leaving group” (LG) is selected from C₁₋₄ alkyl sulfonate, C₆₋₁₀ aryl sulfonate or nitro. More preferably, the Leaving Group (LG) is mesylate, tosylate, nosylate or nitro. Even more preferably, the Leaving Group (LG) is mesylate or nitro, most preferably mesylate.

The term “detecting” as used herein encompasses quantitative and/or qualitative detection.

The compounds of the present invention can be used as an analytical reference or an in vitro screening tool.

For example, the non-labelled compounds of formula (I) according to of the present invention can be used as an analytical reference for the quality control and release of a corresponding labelled compound of the present invention, for example a corresponding ¹⁸F labelled compound of Formula (IV), or a subformulae thereof. This quality control is conducted in an in vitro method.

The compounds of the present invention can be used as an in vitro screening tool for characterization of tissue with TDP-43 pathology and for testing of compounds targeting TDP-43 pathology on such tissue.

The preferred definitions given in the “Definition”-section apply to all of the embodiments described below unless stated otherwise. Various embodiments of the invention are described herein, it will be recognized that features specified in each embodiment may be combined with other specified features to provide further embodiments of the present invention.

### DETAILED DESCRIPTION OF THE INVENTION

Various embodiments of the invention are described herein, it will be recognized that features specified in each embodiment may be combined with other specified features to provide further embodiments of the present invention.

It is understood that all of the definitions which are given with respect to the formula (I) apply to all of the subgenuses thereof, including the formulae (I-a), (I-b), (II), (II′), (III), (IIIa), (IIIb), (IV), and (IVa).

In a first aspect, the present invention relates to a compound having the formula (I)

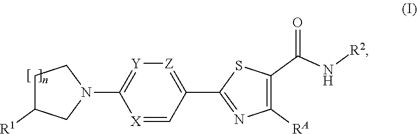

or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N;
R^{A} is H or F; and
R² is a 5- or 6-membered carbocyclic or heterocyclic ring which can be optionally substituted with F, NH₂, CN and/or CH₃, wherein the heterocyclic ring contains one or more (preferably one or two more preferably one) heteroatoms selected from N, O and/or S,
and to compounds having the subformula (Ia)

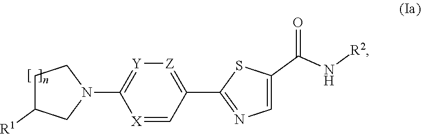

or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.
or the subformula (Ib)

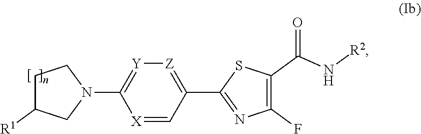

or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.

The present invention relates to a compound of formula (I) wherein R^{A} is H or F. In some preferred embodiments the present invention relates to a compound of formula (I) wherein R^{A} is H (subformula (Ia)). In other preferred embodiments the present invention relates to a compound of formula (I) wherein R^{A} is F (subformula (Ib)).

The present invention relates to a compound of formula (I), or subformula (Ia) or (Ib), wherein n is 1 or 2. In a preferred embodiment n is 1. In another embodiment n is 2.

The present invention relates to a compound of formula (I), or subformula (Ia) or (Ib), wherein R¹ is H or F. In a preferred embodiment R¹ is H. In another most preferred embodiment R¹ is F.

The present invention relates to a compound of formula (I), or subformula (Ia) or (Ib), wherein n is 1, and wherein

| | | |
|---|---|---|
| X is N; | Y is CH; | and Z is CH; or |
| X is N; | Y is CH; | and Z is N; or |
| X is N; | Y is N; | and Z is CH; or |
| X is CH; | Y is CH; | and Z is CH. |

The present invention refers to a compound of formula (I), or subformula (Ia) or (Ib), wherein R² is a 5- or 6-membered carbocyclic or heterocyclic ring which can be optionally substituted with F, NH₂, CN and/or CH₃. Thus, one or more of the same or different substituents can be present. The substituents can be present at any available position. The number of substituents is not particularly limited and can range from 1 to the maximum number of available positions. Preferably the number of substituents, if present, is one or two. The heterocyclic ring contains one or more heteroatoms selected from N, O and S, preferably N or S. The heteroatoms can be the same or different.

In different embodiments, R² is
a 5- or 6-membered carbocyclic ring, preferably an aryl ring, which can be optionally substituted with F, NH₂, CN and/or CH₃,
a 5-membered heteroaryl ring which can be optionally substituted with F, NH₂, CN and/or CH₃, wherein the 5-membered heteroaryl ring contains one or more heteroatoms selected from N, O and/or S, or
a 6-membered heteroaryl ring which can be optionally substituted with F, NH₂, CN and/or CH₃, wherein the 6-membered heteroaryl ring contains one or two heteroatoms selected from O, N and/or S.

Preferred examples of the ring of R² are given in the Definitions section above. Preferably, R² is phenyl, pyridyl, pyrimidinyl, pyrazinyl, pyrazolyl or isothiazolyl, any of which can be optionally substituted with F, NH₂, CN and/or CH₃, e.g., by F, NH₂ and/or CH₃, preferably by F and optionally by NH₂. In one preferred embodiment the phenyl is substituted by F and can optionally be substituted by NH₂ and/or CN. In one preferred embodiment the pyrimidinyl is substituted by F. In another embodiment the pyrimidinyl is unsubstituted. In one preferred embodiment the pyridyl is unsubstituted. In a preferred embodiment, the pyridyl is substituted by F. In one preferred embodiment the isothiazolyl is unsubstituted. In one preferred embodiment the pyrazinyl is substituted by F.

In one embodiment, the present invention relates to a compound of formula (I), or subformula (Ia) or (Ib), wherein R² is a 5- or 6-membered carbocyclic or heterocyclic ring which can be optionally substituted with F, NH₂ and/or CH₃, wherein the heterocyclic ring contains one or more heteroatoms selected from N, O and S. Preferably the 5- or 6-membered carbocyclic or heterocyclic ring is substituted with one or more of F, NH₂ and/or CH₃.

In a preferred embodiment, R² is a 6-membered heteroaryl ring substituted with F and/or NH₂ wherein the 6-membered heteroaryl ring contains at least one heteroatom which is N. In a preferred embodiment, R² is a pyridinyl ring which is substituted with F.

In an embodiment, the present invention relates to a compound of formula (I), or subformula (Ia) or
R² is

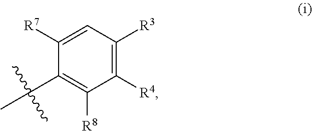

wherein

| | | | |
|---|---|---|---|
| R³ is F, | R⁴ is NH₂, | R⁷ is H, and | R⁸ is H; or |
| R³ is NH₂, | R⁴ is F, | R⁷ is H, and | R⁸ is H; or |
| R³ is CN, | R⁴ is NH₂, | R⁷ is H, and | R⁸ is H; or |
| R³ is H, | R⁴ is NH₂, | R⁷ is H, and | R⁸ is CN; or |
| R³ is H, | R⁴ is NH₂, | R⁷ is H, and | R⁸ is F. |

In a preferred embodiment, R³ is F, R⁴ is NH₂, R⁷ is H, and R⁸ is H.

In another embodiment, the present invention relates to a compound of formula (I), or subformula (Ia) or (Ib), wherein
R² is

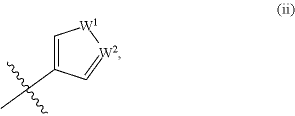

wherein W₁ is N, S or O and W₂ is N.

In another embodiment, the present invention relates to a compound of formula (I), wherein R² is

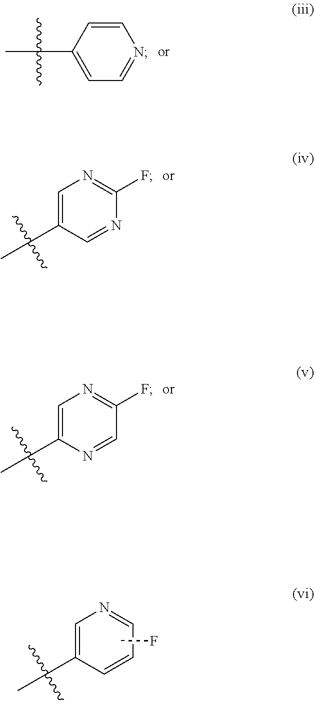

wherein

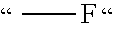

indicates that “F” may be present or absent; or

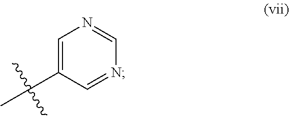 [SMILES: *c1cncnc1]

In one embodiment, the compound of formula (I) or subformula (Ia) or (Ib), is defined as n is 1; R¹ is F; and X, Y and Z are independently CH or N; preferably at least one of X or Y is N, the other is CH; and Z is CH; and R² is

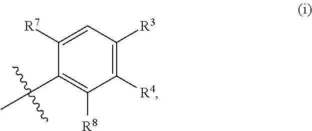

wherein R³ is F, R⁴ is NH₂, R⁷ and R⁸ are H or CH₃, preferably H.

In one embodiment, the compound of formula (I), or subformula (Ia) or (Ib), is defined as n is 1; R¹ is F; X, Y and Z are independently CH or N; preferably at least one of X or Y (preferably X) is N, the other is CH; and Z is CH; and R² is

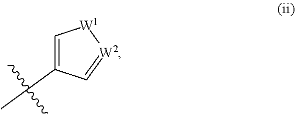

wherein W₁ is S and W₂ is N.

In one embodiment, the compound of formula (I), or subformula (Ia) or (Ib), is defined as n is 1; R¹ is F; X, Y and Z are independently CH or N; preferably at least two of X, Y and Z are N, the other is CH (preferably X and Y are N and Z is CH); and R² is

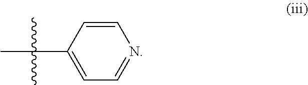 [SMILES: *c1ccncc1]

In one embodiment, the compound of formula (I), or subformula (Ia) or (Ib), is defined as n is 1; R¹ is F; X, Y and Z are independently CH or N; preferably at least one of X or Y (preferably X) is N, the other is CH; and Z is CH; and R² is

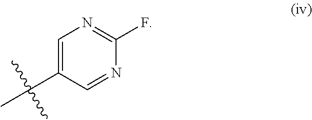 [SMILES: *c1cnc(F)nc1]

In one embodiment, the compound of formula (I), or subformula (Ia) or (Ib), is defined as n is 1; R¹ is F; X, Y and Z are independently OH or N; preferably at least one of X or Y (preferably X) is N, the other is CH; and Z is CH; and R² is

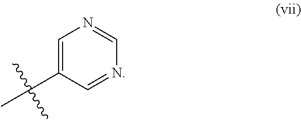 [SMILES: *c1cncnc1]

In one embodiment, the compound of formula (I), or subformula (Ia) or (Ib), is defined as n is 1; R¹ is F; X, Y and Z are independently CH or N; preferably at least one of X or Y (preferably X) is N, the other is CH; and Z is CH; and R² is

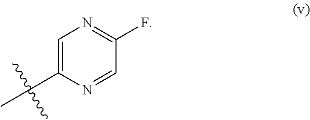 [SMILES: *c1cnc(F)cn1]

In one embodiment, the compound of formula (I), or subformula (Ia) or (Ib), is defined as n is 1; R¹ is F; X, Y and Z are independently CH or N; preferably at least one of X or Y (preferably X) is N, the other is CH; and Z is CH; or X, Y and Z are CH; and R² is

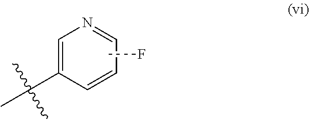

wherein

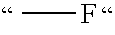

indicates that “F” may be present or absent.

Preferred compounds of formula (I) include:

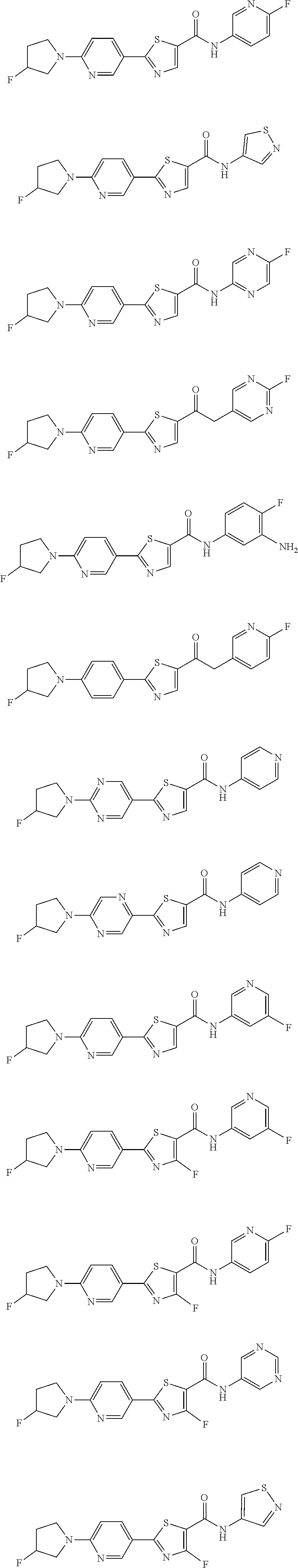

In one embodiment preferred compounds of formula (I) can be selected from the following stereoisomers:

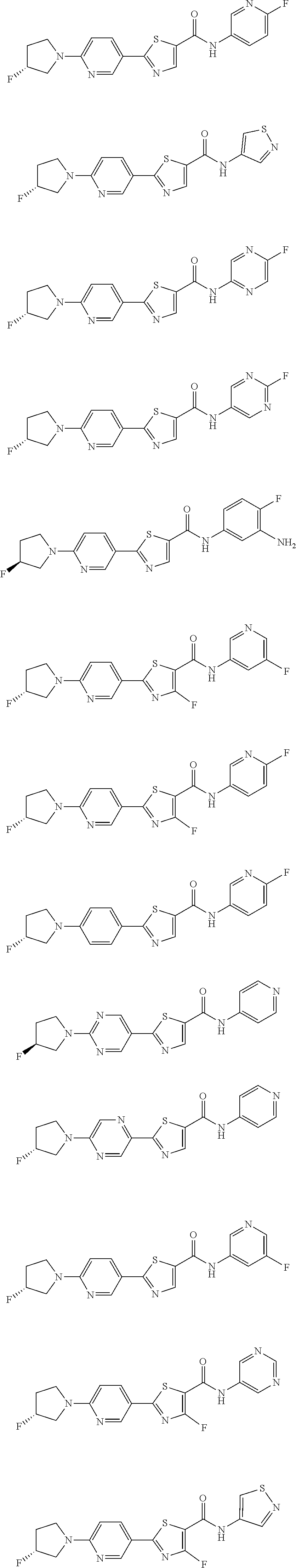

In one embodiment, the present invention relates to a compound of formula (I), or subformula (Ia) or (Ib), which comprises at least one detectable label, which means, the compound of formula (I), or subformula (Ia) or (Ib), comprises one or more detectable labels.

The type of the detectable label is not specifically limited and will depend on the detection method chosen. Examples of possible detectable labels include isotopes such as radioisotopes (namely radionuclides), in particular positron emitters or gamma emitters. The detectable label such as the radioisotope, in particular the positron emitter or gamma emitter, should be present in an amount, which is not identical to the natural amount of the respective isotope. Furthermore, the employed amount should allow detection thereof by the chosen detection method.

In a preferred embodiment, the detectable label is selected from ³H and/or ¹⁸F, most preferably ¹⁸F. The detectable label can be present at any available position. Typically, the detectable label is a radioactive isotope of one of the atoms which are present in the compound of formula (I). For instance, any reference to “F” in the present invention covers ¹⁹F (stable) or ¹⁸F (detectable label). Any reference to “H” covers ¹H (stable) or ³H (detectable label, so called tritium and represented herein as “T”).

Isotopic variations of the compounds of the invention can generally be prepared by conventional procedures such as by the illustrative methods or by the preparations described in the Examples and Preparative Examples hereinafter using appropriate isotopic variations of suitable reagents, commercially available or prepared by known synthetic techniques. Radioisotopes, in particular positron emitters or gamma emitters, can be included into the compounds of the invention by methods which are usual in the field of organic synthesis. Typically, they will be introduced by using a correspondingly labeled starting material. Illustrative methods of introducing detectable labels are described, for instance, in U.S. Pat. No. 8,932,557 which is incorporated herein by reference. ¹⁸F can be attached at any position which is suitable for attaching a fluorine. ¹⁸F-labeled compounds are particularly suitable for imaging applications such as positron emission tomography (PET). The corresponding compounds which include natural fluorine isotope ¹⁹F are also of particular interest as they can be used as analytical standards and references during manufacturing, quality control, release, and clinical use of their ¹⁸F-analogs.

In the compounds having the formula (I), ¹⁸F can be present, for instance, as the F substituent of R² or as R¹. Preferably it is present as R¹ (R¹ is ¹⁸F).

If ³H is employed as a detectable label it is preferably attached in the form of —CT₃ (T means ³H) at any position at which a CH₃ group can be attached. Substitution with radioisotopes such as ³H may afford certain diagnostic advantages resulting from greater metabolic stability by reducing, for example, defluorination, increasing in vivo half-life or reducing dosage requirements, while keeping or improving the original compound efficacy.

In one embodiment, the present invention relates to Tritium (³H) detectably labeled compounds having the formula (I), and subformulae (la) or (Ib), as described above, wherein at least one Hydrogen (H) is replaced by a detectable label selected from Tritium (³H). Tritium (³H) detectably labeled compounds having the formula (I) are preferably defined wherein 1 to 3 Hydrogens (H) are replaced by Tritium (³H). Tritium (³H) detectably labeled compounds having the formula (I) are more preferably defined wherein 2 or 3 Hydrogens (H) are replaced by Tritium (³H). Tritium (³H) detectably labeled compounds having the formula (I), and subformulae (la) or (Ib), are even more preferably defined wherein 2 Hydrogens (H) are replaced by Tritium (³H).

In one embodiment, the present invention relates to Tritium (³H) detectably labeled compounds having the formula (I-T)

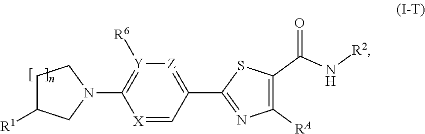

or a stereoisomer, polymorph, racemic mixture, tautomer, pharmaceutically acceptable salt, hydrate, or solvate thereof, or mixtures thereof; wherein n, R^{A}, R¹, R², X and Z are as defined herein with respect to compound of formula (I); Y is CR⁶, R⁶ is T or H, and wherein R² is substituted by at least one CT₃ and/or at least one of the hydrogen atoms in R² is replaced by T. T is ³H.

In one embodiment, the present invention relates to Tritium (³H) detectably labeled compounds having the formula (I-Ta)

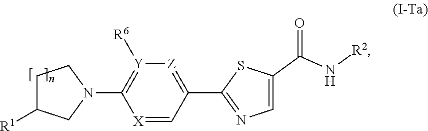

or a stereoisomer, polymorph, racemic mixture, tautomer, pharmaceutically acceptable salt, hydrate, or solvate thereof, or mixtures thereof; wherein n, R¹, R², X and Z are as defined herein with respect to compound of formula (I); Y is CR⁶, R⁶ is T or H, and wherein R² is substituted by at least one CT₃ and/or at least one of the hydrogen atoms in R² is replaced by T. T is ³H.

In another embodiment, the present invention relates to Tritium (³H) detectably labeled compounds having the formula (I-Tb)

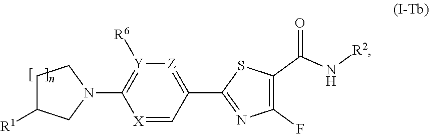

or a stereoisomer, polymorph, racemic mixture, tautomer, pharmaceutically acceptable salt, hydrate, or solvate thereof, or mixtures thereof; wherein n, R¹, R², X and Z are as defined herein with respect to compound of formula (I); Y is CR¹, R⁶ is T or H, and wherein R² is substituted by at least one CT₃ and/or at least one of the hydrogen atoms in R² is replaced by T. T is ³H.

In some embodiments R⁶ is T. In other embodiments R⁶ is H.

In a preferred embodiment, at least one of the hydrogen atoms in R² is replaced by T. In a preferred embodiment, R⁶ is T and at least one of the hydrogen atoms in R² is replaced by T.

In a preferred embodiment, the present invention relates to a compound of formula (I-T), wherein wherein
R² is

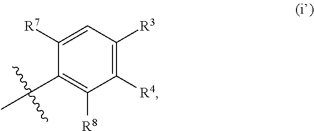

wherein R³ is F, R⁴ is —NH₂, and at least one of R⁷ and R⁸ is T and, if applicable, the other is H; preferably R⁷ and R⁸ are T; and R⁶ is T; or wherein
R² is

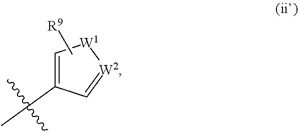

wherein W¹ is S and W² is N and R⁹ is T and R⁶ is T; or wherein
R² is

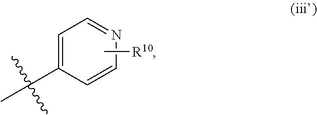

wherein R¹⁰ is T and R⁶ is T; or wherein
R² is

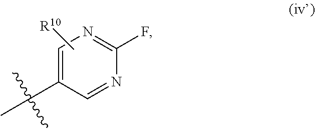

wherein R¹⁰ is T and R⁶ is T; or wherein
R² is

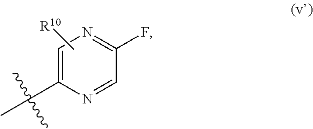

wherein R¹⁰ is T and R⁶ is T; or wherein
R² is

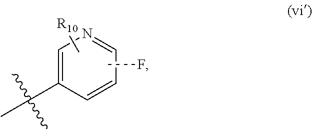

wherein

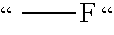

indicates that “F” may be present or absent, and wherein R¹⁰ is T and R⁶ is T; or wherein
R² is

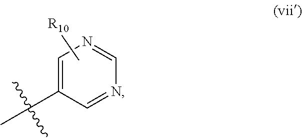

wherein R¹⁰ is T and R⁶ is T; or wherein
R² is

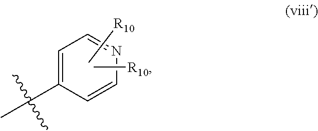

wherein R¹⁰ is T and R⁶ is H; or wherein;
R² is

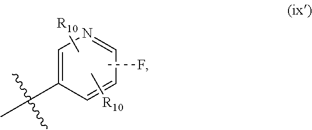

R¹⁰, wherein

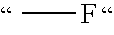

indicates that “F” may be present or absent, and wherein R¹⁰ is T and R⁶ is H.

In one embodiment, the present invention relates to a compound of formula (I-T), wherein
R¹ is H;
R⁶ is T; and
R² is

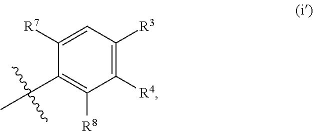

wherein R³ is F; R⁴ is —NH₂; and at least one of R⁷ and R⁸ is T and, if applicable, the other is H. Preferably R⁷ is T; and R⁸ is T.

In one embodiment, the present invention relates to a compound of formula (I-T) wherein
R¹ is F;
R⁶ is T; and
R² is

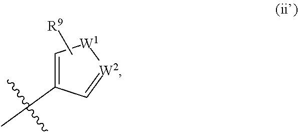

wherein W¹ is S and W² is N and R⁹ is T.

In one embodiment, the present invention relates to a compound of formula (I-T), wherein
R¹ is H or F;
R⁶ is T; and
R² is

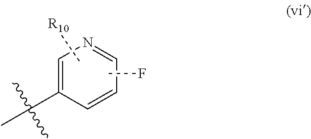

wherein R¹⁰ is T.

In a preferred embodiment, the present invention relates to a compound of formula (I-T)

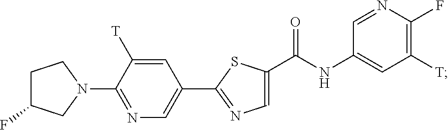

wherein T is ³H.

Preferred Tritium (³H) detectably labeled compounds of formula (I-T) according to present invention include (wherein T means ³H):

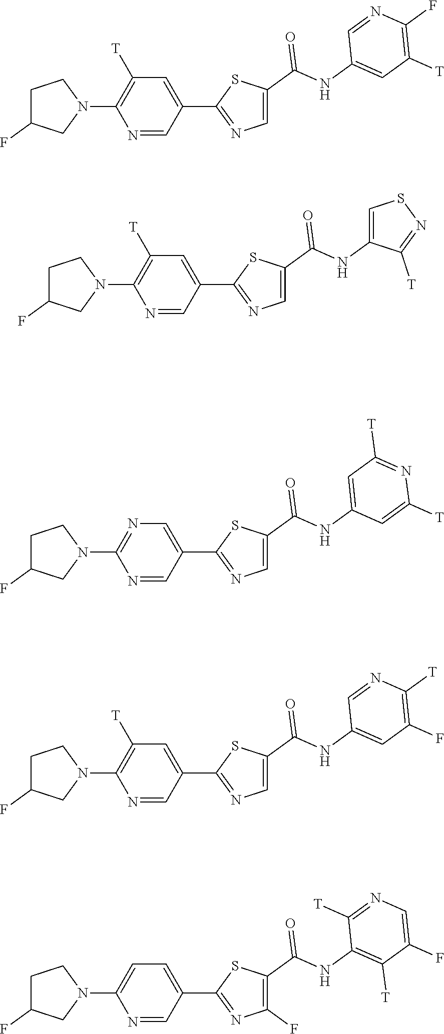

More preferably, the Tritium (³H) detectably labeled compound for formula (I-T) according to the present invention can be the stereoisomer (wherein T means ³H)

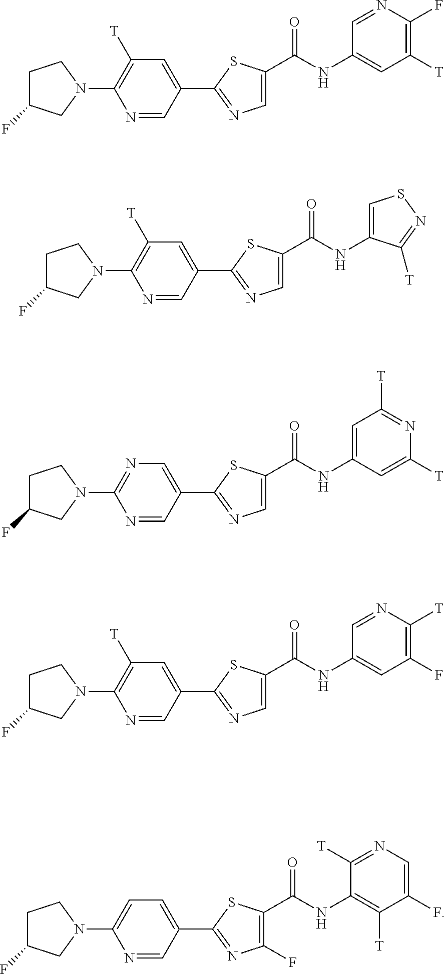

In one embodiment, the present invention provides a ¹⁸F detectably labeled compound of formula (I-F)

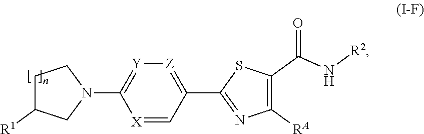

or a stereoisomer, polymorph, racemic mixture, tautomer, pharmaceutically acceptable salt, hydrate, or solvate thereof, or mixtures thereof;
wherein
R^{1′} is ¹⁸F; and n, X, Y, Z, R^{A} and R² are as defined herein with respect to compound of formula (I) and preferably n is 1.

In one embodiment, the ¹⁸F detectably labeled compound of formula (I-F) has the formula (I-Fa):

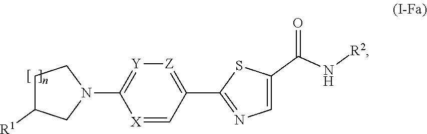

or a stereoisomer, polymorph, racemic mixture, tautomer, pharmaceutically acceptable salt, hydrate, or solvate thereof, or mixtures thereof;
wherein
R^{1′} is ¹⁸F; and n, X, Y, Z and R² are as defined herein with respect to compound of formula (I) and preferably n is 1.

In one embodiment, ¹⁸F detectably labeled compound of formula (I-F) has the formula (I-Fb):

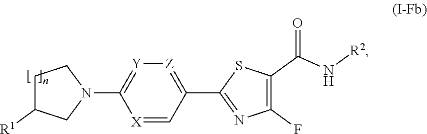

or a stereoisomer, polymorph, racemic mixture, tautomer, pharmaceutically acceptable salt, hydrate, or solvate thereof, or mixtures thereof;
wherein
R¹ is ¹⁸F; and n, X, Y, Z and R² are as defined herein with respect to compound of formula (I) and preferably n is 1.

In one embodiment, (I-F) is the following compound (R¹ is ¹⁸F (detectable label);

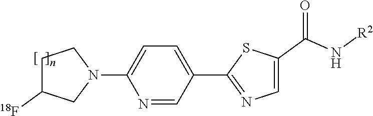

wherein n and R² are as defined herein with respect to compound of formula (I).

In another embodiment, (I-F) is the following compound

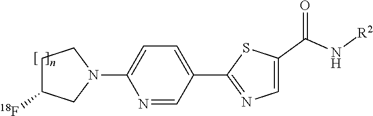

wherein n and R² are as defined herein with respect to compound of formula (I).

In preferred embodiments of Formula (I-F), (I-Fa) or (I-Fb), n is 1 or 2; and
R² is

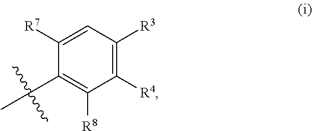

wherein R³ is F, R⁴ is NH₂, R¹ is H, and R⁸ is H; or wherein
R² is

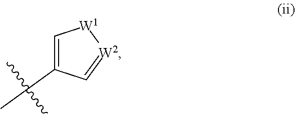

wherein W₁ is S and W₂ is N; or wherein
R² is

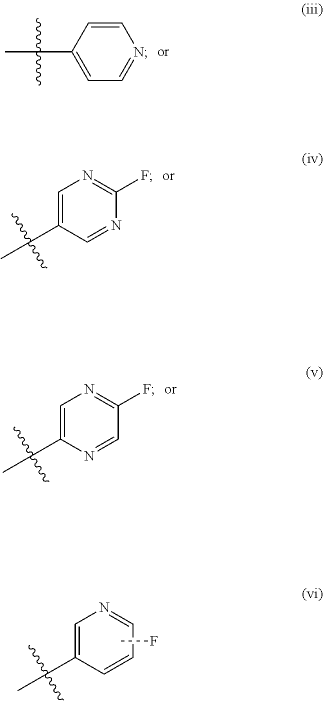

wherein

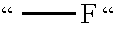

indicates that “F” may be present or absent; or

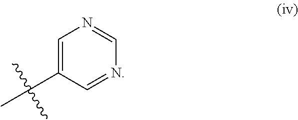 [SMILES: *c1cncnc1]

In a preferred embodiment, the present invention relates to a compound of formula (I-F), wherein
n is 1 or 2;
R¹ is ¹⁸F (detectable label);
R² is

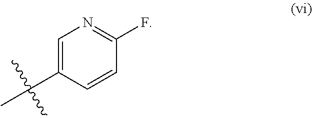 [SMILES: *c1ccc(F)nc1]

In a preferred embodiment n is 1.

A preferred ¹⁸F detectably labeled compound of formula (I-F) according to the present invention is

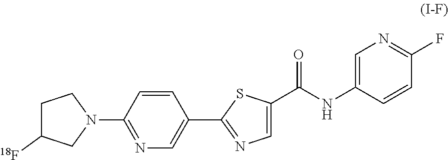 [SMILES: O=C(Nc1ccc(F)nc1)c1cnc(-c2ccc(N3CCC([18F])C3)nc2)s1]

More preferably, the ¹⁸F detectably labeled compound of formula (I-F) according to the present invention can be the stereoisomer

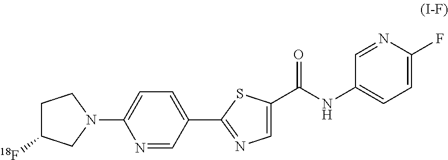 [SMILES: O=C(Nc1ccc(F)nc1)c1cnc(-c2ccc(N3CC[C@@H]([18F])C3)nc2)s1]

### Diagnostic Compositions

In a second aspect, the present invention relates to a diagnostic composition comprising a compound of formula (I), as described above, and optionally at least one physiologically acceptable carrier, diluent, adjuvant and/or excipient.

The compounds of the present invention are particularly suitable for imaging TDP-43 aggregates.

The imaging can be conducted in mammals, preferably in humans. The imaging is preferably in vitro imaging, ex vivo imaging, or in vivo imaging. More preferably the imaging is in vivo imaging. Even more preferably, the imaging is brain imaging. The imaging can also be eye/retinal imaging or imaging of tissue of the central nervous system.

The compounds or composition of the present invention are particularly suitable for use in diagnostics. The diagnostics can be conducted for mammals, preferably for humans. The tissue of interest on which the diagnostics is conducted can be brain, tissue of the central nervous system, tissue of the eye (such as retinal tissue) or other tissues, or body fluids such as cerebrospinal fluid (CSF). The tissue is preferably brain tissue.

A “diagnostic composition” is defined in the present invention as a composition comprising one or more compounds of the present invention in a form suitable for administration to a patient (e.g., a mammal such as a human), and which is suitable for use in the diagnosis of the specific disease, disorder or abnormality at issue. In one embodiment, the diagnostic composition comprises a detectably labeled compound of the invention as described above and optionally at least one physiologically acceptable carrier, diluent, adjuvant and/or excipient.

Preferred detectably labeled compounds of the invention are of formula (I-T) or subformulae (l-Ta) and (I-Tb), or (I-F) or subformulae (I-Fa) and (I-Fb).

The diagnostic composition or the compounds are suitable for use in the imaging of TDP-43 aggregates, particularly by positron emission tomography.

The diagnostic composition is suitable for use in the diagnosis of a disease, disorder or abnormality associated with TDP-43 aggregates or a TDP-43 proteinopathy, particularly wherein the diagnosis is conducted by positron emission tomography. as defined herein below.

Preferably a diagnostic composition further comprises a physiologically acceptable excipient, carrier, diluent, or adjuvant. Administration is preferably carried out as defined below, more preferably by injection of the composition as an aqueous solution. The diagnostic composition may optionally contain further ingredients such as buffers; pharmaceutically acceptable solubilizers (e.g., cyclodextrins or surfactants such as Pluronic, Tween or phospholipids); and pharmaceutically acceptable stabilizers or antioxidants (such as ascorbic acid, gentisic acid or para-aminobenzoic acid). The dose of the compound of the invention will vary depending on the exact compound to be administered, the weight of the patient, and other variables as would be apparent to a physician skilled in the art.

While it is possible for the compounds of the invention to be administered alone, it is preferable to formulate them into a diagnostic composition in accordance with standard pharmaceutical practice.

Thus, a diagnostic composition which comprises a diagnostically effective amount of a compound of the invention in combination with a pharmaceutically acceptable carrier, diluent, adjuvant and/or excipient is part of the invention. The preferred pharmaceutically acceptable carrier, diluent, adjuvant and/or excipient is one that is physiologically compatible with the diagnostic composition according to the present invention.

Pharmaceutically acceptable excipients are well known in the pharmaceutical art, and are described, for example, in Remington's Pharmaceutical Sciences, 18^{th} Ed. (Alfonso R. Gennaro, ed.; Mack Publishing Company, Easton, PA, 1990). The pharmaceutically acceptable excipient can be selected with regard to the intended route of administration and standard pharmaceutical practice. The excipient must be acceptable in the sense of being not deleterious to the recipient thereof.

Pharmaceutically useful excipients, carriers, adjuvants and diluents that may be used in the formulation of the diagnostic composition of the present invention may comprise, for example, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate, binders, adjuvants, solubilizers, thickening agents, stabilizers, disintegrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-g-cyclodextrin, polyvinylpyrrolidone, low melting waxes, and ion exchange resins.

The routes for administration (delivery) of the compounds of the invention include, but are not limited to, one or more of: intravenous, gastrointestinal, intraspinal, intraperitoneal, intramuscular, oral (e. g. as a tablet, capsule, or as an ingestible solution), topical, mucosal (e. g. as a nasal spray or aerosol for inhalation), nasal, parenteral (e. g. by an injectable form), intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. Preferably, the route for administration (delivery) of the compounds of the invention is parenteral.

If the compounds of the present invention (for instance, detectably labeled compounds such as those with a 3H or ¹⁸F detectable label) are administered parenterally, then examples of such routes of administration include one or more of: intravenously, intraarterially, intraperitoneally, intrathecally, intraventricularly, intraurethrally, intrasternally, intracranially, intramuscularly or subcutaneously and/or using infusion techniques. For parenteral administration, the compounds are best used in the form of a sterile aqueous solution which may contain other excipients. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Typically, a physician will determine the actual dosage which will be most suitable for an individual patient. The dose of the compounds of the present invention (for instance, detectably labeled compounds such as those with a 3H or ¹⁸F detectable label) will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

Due to their design and their binding characteristics, the compounds of the present invention, as defined herein, can be used in the diagnosis of diseases, disorders and abnormalities associated with TDP-43 aggregates. The compounds of the present invention are particularly suitable for positron emission tomography imaging of TDP-43 aggregates.

The compounds or the diagnostic composition or the methods of the present invention, as disclosed herein, are particularly suitable for use in the diagnosis of diseases, disorders or abnormalities associated with TDP-43 aggregates or the TDP-43 proteinopathy, such as disease, disorder or abnormality selected from, but not limited to, Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with ubiquitin-positive TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

In one embodiment, the diseases, disorders or abnormalities associated with TDP-43 aggregates or the TDP-43 proteinopathy is amyotrophic lateral sclerosis (ALS).

In one embodiment, the diagnosis of diseases, disorders or abnormalities associated with TDP-43 aggregates or the TDP-43 proteinopathy is Alzheimer's disease (AD).

In one embodiment, the diagnosis of diseases, disorders or abnormalities associated with TDP-43 aggregates or the TDP-43 proteinopathy is Frontotemporal dementia (FTD) including Frontotemporal lobar dementia TDP-43 or Frontotemporal lobar degeneration with TDP-43 inclusions (FTLD-TDP).

In one embodiment, the diagnosis of diseases, disorders or abnormalities associated with TDP-43 aggregates or the TDP-43 proteinopathy is limbic-predominant age-related TDP-43 encephalopathy (LATE).

### Methods and Uses

In a third aspect, the present invention relates to the methods and uses as listed below
A method of diagnosing a disease, disorder or abnormality associated with TDP-43 aggregates or a TDP-43 proteinopathy, or a predisposition thereto, in a subject;
A method of positron emission tomography (PET) imaging of TDP-43 aggregates in a tissue of a subject;
A method for the detection and optional quantification of TDP-43 aggregates in a tissue of a subject;
A method of determining an amount of TDP-43 aggregates in a sample or a specific body part or body area;
A method of collecting data for the diagnosis of a disease, disorder or abnormality associated with TDP-43 aggregates or for the diagnosis of a TDP-43 proteinopathy;
A method of collecting data for determining a predisposition to a disease, disorder or abnormality associated with TDP-43 aggregates or a TDP-43 proteinopathy;
A method of collecting data for monitoring the progression of a disease, disorder or abnormality associated with TDP-43 aggregates or for monitoring the progression of a TDP-43 proteinopathy in a patient,
A method of collecting data for predicting responsiveness of a patient suffering from a disease, disorder or abnormality associated with TDP-43 aggregates or suffering from a TDP-43 proteinopathy to a treatment with a medicament;
Use of a compound of the invention as a TDP-43 aggregates' biomarker or a TDP-43 proteinopathy biomarker,
Use of a compound of the invention as a TDP-43 proteinopathy diagnostic agent or diagnostic tool,
Use of a compound of the invention as an in vitro analytical reference or an in vitro screening tool.

Any of the compounds of the present invention (e.g. compound of formula (I-A) to (I-J), (I′-A) to (I′-J), (I-T), and (I-F) can be used in the above summarized methods. Preferably, said compounds are detectably labeled compounds (e.g. such as those with a ³H or ¹⁸F detectable label). The compounds of formulae (II), (III), and (IV) are precursors of the compounds of formula (I).

The methods of the invention can include the step of bringing a sample, a specific body part or a body area suspected to contain TDP-43 aggregates into contact with a compound of the invention or a diagnostic composition which comprises a compound according to the invention.

The body is preferably of a mammal, more preferably of a human, including the full body or partial body area/part of the patient suspected to contain TDP-43 aggregates.

The sample can be selected from tissue or body fluids suspected to contain TDP-43 aggregates, the sample being obtained from the patient. Preferably, the tissue is selected from tissue of the central nervous system (CNS), eye tissue or brain tissue, more preferably brain tissue. Examples of body fluids include cerebrospinal fluid (CSF) or blood. The sample can be obtained from a mammal, more preferably a human. Preferably, the sample is an in vitro sample from a patient.

An in vitro sample or a specific body part or body area obtained from a patient can be brought into contact with a compound of the invention by direct incubation.

In an in vivo method, the specific body part or body area can be brought into contact with a compound of the invention by administering an effective amount of a compound of the invention to the patient. The effective amount of a compound of the invention is an amount which is suitable for allowing the presence or absence of TDP-43 aggregates in the specific body part or body area to be determined using the chosen analytical technique.

The step of allowing the compound of the invention to bind to the TDP-43 aggregates includes allowing sufficient time for said binding to happen. The amount of time required for binding will depend on the type of test (e.g., in vitro or in vivo) and can be determined by a person skilled in the field by routine experiments. In an in vitro method the amount of time will depend on the sample or specific body part or body area and can range, for instance, from about 30 minutes to about 120 minutes. In an in vivo method, the amount of time will depend on the time which is required for the compound of the invention to reach the specific body part or body area suspected to contain TDP-43 aggregates. The amount of time should not be too long to avoid washout and/or metabolism of the compound of the invention. The duration can range, for instance, from about 0 minutes to about 240 minutes (which is the duration of a PET scan during initial compound characterization (NHP PET and later FiH-study)).

The method of detecting the compound of the invention bound to the TDP-43 aggregates is not particularly limited and depends, among others, on the detectable label, the type of sample, specific body part or body area and whether the method is an in vitro or in vivo method. Possible detection methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). The fluorescence imaging technique and/or nuclear imaging technique can be employed for monitoring and/or visualizing the distribution of the compound of the invention within the sample or the body. The imaging system is such to provide an image of bound detectably label such as radioisotopes, in particular positron emitters or gamma emitters, as present in the tested sample, the tested specific body part or the tested body area. Preferably, the compound of the invention bound to the TDP-43 aggregates is detected by an imaging apparatus such as PET or a SPECT scanner. The amount of the compound bound to the TDP-43 can be determined by visual or quantitative analysis, for example, using PET scan images.

In one embodiment, the presence or absence of a compound of the invention bound to the TDP-43 aggregates can be correlated with the presence or absence of TDP-43 aggregates in the sample or specific body part or body area. The correlation can be qualitative or quantitative. In a preferred embodiment, this step (d) comprises:
Determining the amount of the compound of the invention bound to the TDP-43 aggregates, for instance, by measuring the radioactive signal of the compound which is bound to the TDP-43 aggregates;
Correlating the amount of the compound of the invention bound to the TDP-43 aggregates with the amount of TDP-43 aggregates in the sample or specific body part or body area; and
Optionally comparing the amount of the compound bound to the TDP-43 aggregates in the sample or specific body part or body area to a normal control value in a healthy control subject.

The amount of the compound bound to the TDP-43 aggregates can be determined by any appropriate method. A preferred method is positron emission tomography (PET).

In another embodiment, the presence or absence of the compound of the invention bound to TDP-43 aggregates can be correlated with the disease, disorder or abnormality associated with TDP-43 aggregates or with the TDP-43 proteinopathy or a predisposition thereto. The correlation can be qualitative or quantitative. In a preferred embodiment, this step (d) comprises:
Determining the amount of the compound of the invention bound to the TDP-43 aggregates; for instance by measuring the radioactive signal of the compound, which is bound to the TDP-43 aggregates;
Correlating the amount of the compound of the invention bound to the TDP-43 aggregates with the amount of TDP-43 aggregates in the sample or specific body part or body area; and
Optionally comparing the amount of the compound bound to the TDP-43 aggregates in the sample or specific body part or body area to a normal control value in a healthy control subject.

In any of the methods disclosed herein, steps (a) to (c) and, if present, optional steps (d) and (e), can be repeated at least one time. The repetition of the steps is particularly useful in the method of collecting data for monitoring the progression and the method of collecting data for predicting responsiveness. In these methods, it may be expedient to monitor the patient over time and repeat the above steps after a certain period of time has elapsed. The time interval before the above-mentioned steps are repeated can be determined by a physician depending on the severity of the disease, disorder or abnormality associated with TDP-43 aggregates or the TDP-43 proteinopathy.

In one embodiment, the present invention relates to a method of detecting a neurological disease, disorder or abnormality associated with TDP-43 aggregates or a TDP-43 proteinopathy, or a predisposition thereto, in a subject, the method comprising the steps:
(a) Administering a compound of the invention, or a diagnostic composition comprising a compound of the invention as disclosed herein, to the subject;
(b) Allowing the compound to bind to the TDP-43 aggregates; and
(c) Detecting the compound which is bound to the TDP-43 aggregates.

In one embodiment, the present invention relates to a method (e.g. in vivo or in vitro method) for the detection and optional quantification of TDP-43 aggregates in a tissue of a subject, the method comprising the steps:
(a) Administering a compound of the invention as disclosed herein; or a diagnostic composition comprising a compound of the invention as disclosed herein, to the subject;
(b) Allowing the compound to bind to the TDP-43 aggregates; and
(c) Detecting and optionally quantifying the compound bound to the TDP-43 aggregates using positron emission tomography.

In one embodiment, the present invention relates to a method of the diagnostic imaging of the brain of a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates by collecting a positron emission tomography (PET) image of the brain of the subject.

### Imaging:

The present invention relates to a method of imaging TDP-43 aggregates using the compounds of the invention. Imaging can be conducted, for example, using any of the above-mentioned methods, particularly by PET.

In one embodiment, the present invention relates to a method of imaging TDP-43 aggregates in a sample or a patient, in particular in a brain or a sample taken from the patient's brain, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates.

In one embodiment, the present invention relates to a method of imaging or diagnosing a disease, disorder or abnormality associated with TDP-43 aggregates or a TDP-43 proteinopathy, or a predisposition thereto, in a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates in the brain of the subject.

In one embodiment, the present invention relates to a method of diagnosing a disease, disorder or abnormality associated with TDP-43 aggregates or a TDP-43 proteinopathy, or a predisposition thereto, in a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates.

In one embodiment the method of diagnosing a disease, disorder or abnormality associated with TDP-43 aggregates or a TDP-43 proteinopathy, or a predisposition thereto, further comprises the steps of:
(d) Generating an image representative of the location and/or amount of the compound bound to the TDP-43 aggregates,
(e) Optionally comparing the generated image with the control image of a healthy control subject, wherein an increase in binding signal indicates that the subject is suffering from or at risk of developing a disease, disorder or abnormality associated with TDP-43 aggregates or a TDP-43 proteinopathy.

In one embodiment, the present invention relates to a method of diagnosing a disease, disorder or abnormality associated with TDP-43 aggregates or a TDP-43 proteinopathy, or a predisposition thereto, in a subject, the method comprising the steps:
(a) Administering a compound of the invention; or a diagnostic composition comprising a compound of the invention as disclosed herein, to the subject;
(b) Allowing the compound to bind to the TDP-43 aggregates;
(c) Detecting the compound bound to the TDP-43 aggregates; and
(d) Generating an image representative of the location and/or amount of the compound bound to the TDP-43 aggregates.

In one embodiment, the present invention relates to a method of positron emission tomography (PET) imaging of TDP-43 aggregates in a tissue of a subject, the method comprising the steps:
(a) Administering a compound of the invention; or a diagnostic composition comprising a compound of the invention as disclosed herein, to the subject;
(b) Allowing the compound to bind to the TDP-43 aggregates; and
(c) Detecting the compound bound to the TDP-43 aggregates by collecting a positron emission tomography (PET) image of the tissue of the subject.

Preferably, the tissue is a tissue of the central nervous system (CNS), an eye tissue or a brain tissue.

More preferably, the tissue is brain tissue.

In one embodiment, the present invention relates to a method of imaging TDP-43 aggregates in a sample or a patient, the method comprising the steps:
(a) Bringing a sample, a specific body part or body area suspected to contain TDP-43 aggregates into contact with a compound of the invention; or with a diagnostic composition comprising a compound of the invention as disclosed herein;
(b) Allowing the compound to bind to the TDP-43 aggregates; and
(c) Detecting the compound bound to the TDP-43 aggregates by imaging the sample, the specific body part or the body area with an imaging system.

In one embodiment, the present invention relates to a method for imaging TDP-43 aggregates in an in vitro sample of a patient, the method comprising the steps:
(a) Bringing the in vitro sample suspected to contain TDP-43 aggregated into contact with a compound of the invention; or with a diagnostic composition comprising a compound of the invention as disclosed herein;
(b) Allowing the compound to bind to the TDP-43 aggregates; and
(c) Detecting the compound bound to the TDP-43 aggregates by imaging the in vitro sample with an imaging system.

In one embodiment, the present invention relates to a method of imaging TDP-43 aggregates in a patient or a specific body part or a body area of a patient, the method comprising the steps:
(a) Bringing a sample or a specific body part or body area suspected to contain TDP-43 aggregates into contact with a compound of the invention, preferably with a compound of formula (I-T) or subformula (I-Ta) or (I-Tb), or of formula (I-F) or subformula (I-Fa) or (I-Fb); or with diagnostic composition comprising a compound of the invention as disclosed herein, preferably a compound of formula (I-T) or of formula (I-F);
(b) Allowing the compound to bind to the TDP-43 aggregates; and
(c) Detecting the compound bound to the TDP-43 aggregates by imaging the sample or the specific body part or the body area of the patient with an imaging system.

The step of imaging the sample, the patient, the specific body part or the body area of the patient with an imaging system includes detecting the compound of the invention bound to the TDP-43 aggregates using an imaging system as disclosed herein. Detecting the compound of the invention bound to the TDP-43 aggregates allows to identify by imaging the distribution of TDP-43 aggregates in the tested sample, the patient, the specific body part or body area. The PET imaging should be conducted when the compound has penetrated the tissue and the compound has bound to the TDP-43 aggregates.

### Determining the Amount of TDP-43 Aggregates:

In one embodiment, the present invention relates to a method of determining the amount of TDP-43 aggregates in a sample, a specific body part or body area suspected to contain TDP-43 aggregates using a compound of the invention.

In one embodiment the present invention provides a method for determining the amount of TDP-43 aggregates in the sample, the specific body part or the body area suspected to contain TDP-43 aggregates, wherein the method comprises the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates;
(d) Determining the amount of compound of the invention bound to the TDP-43 aggregates by measuring the radioactive signal of the compound; and
(e) Optionally calculating the amount of TDP-43 aggregates in the sample, the specific body part or body area.

The amount of TDP-43 aggregates can for example be calculated by determining the Bmax (the maximal number of binding sites).

A radioactive signal is observed when a detectably labelled compound of the invention, which comprises at least one radiolabeled atom (e.g. ³H, ²H, or ¹⁸F), is bound to the TDP-43 aggregates.

### Diagnosing:

In one embodiment, the present invention relates to a method of diagnosing a disease, disorder or abnormality associated with TDP-43 aggregates or a TDP-43 proteinopathy or a predisposition thereto, the method comprising the steps of:
(c) Detecting the compound of the invention bound to the TDP-43 aggregates; and
(d) Correlating the presence or absence of the compound of the invention bound to TDP-43 aggregates with the disease, disorder or abnormality associated with TDP-43 aggregates or with the TDP-43 proteinopathy.

Preferably, the method of diagnosing the disease, disorder or abnormality associated with TDP-43 aggregates or the TDP-43 proteinopathy or a predisposition thereto comprises the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Correlating the presence or absence of the compound of the invention bound to TDP-43 aggregates with the disease, disorder or abnormality associated with TDP-43 aggregates or with the TDP-43 proteinopathy in the sample or specific body part or body area.

In one embodiment, the present invention relates to a method of collecting data for the diagnosis of a disease, disorder or abnormality associated with TDP-43 aggregates or a TDP-43 proteinopathy or a predisposition thereto, the method comprising the following steps:
(a) Bringing a sample or a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Optionally correlating the presence or absence of the compound of the invention bound to the TDP-43 aggregates with the presence or absence of TDP-43 aggregates in the sample or specific body part or body area.

After the sample or a specific body part or body area has been brought into contact with the compound of the present invention, the compound is allowed to bind to the TDP-43 aggregates. The amount of time required for binding will depend on the type of test (e.g., in vitro or in vivo) and can be determined by a person skilled in the field by routine experiments. The compound which has bound to the TDP-43 aggregates can be subsequently detected by any appropriate method. The specific method chosen will depend on the detectable label which has been chosen. Examples of possible methods include, but are not limited to, a fluorescence imaging technique or a nuclear imaging technique such as positron emission tomography (PET), single photon emission computed tomography (SPECT), magnetic resonance imaging (MRI), and contrast-enhanced magnetic resonance imaging (MRI). The fluorescence imaging technique and/or nuclear imaging technique can be employed for monitoring and/or visualizing the distribution of the detectably labelled compound within the sample or a specific body part or body area.

The step of optionally correlating the presence or absence of the compound bound to the TDP-43 aggregates with the presence or absence of TDP-43 aggregates in the sample or specific body part or body area, as mentioned herein above, comprises the steps of
determining the amount of the compound bound to the TDP-43 aggregates;
correlating the amount of the compound bound to the TDP-43 aggregates with the amount of TDP-43 aggregates in the sample or specific body part or body area; and
optionally comparing the amount of the compound bound to the TDP-43 aggregates in the sample or specific body part or body area to a normal control value in a healthy control subject.

The amount of compound bound to the TDP-43 aggregates can be compared to a normal control value which has been determined in a sample or a specific body part or body area of a healthy subject, wherein an increase in the amount of the compound bound to the TDP-43 aggregates compared to a normal control value may indicate that the patient is suffering from or is at risk of developing a disease, disorder or abnormality associated with TDP-43 aggregates or a TDP-43 proteinopathy.

If the amount of the compound bound to the TDP-43 aggregates is higher than the normal control value, as defined herein, then it can be expected that the patient is suffering from or is likely to suffer from a disease, disorder or abnormality associated with TDP-43 aggregates or TDP-43 proteinopathy.

### Determining a Predisposition:

A further aspect of the present invention relates to a method of collecting data for determining a predisposition to a disease, disorder or abnormality associated with TDP-43 aggregates or a TDP-43 proteinopathy. The method comprises the steps:
(a) Bringing a sample or a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates; and
(d) Optionally correlating the presence or absence of the compound of the invention bound to the TDP-43 aggregates with the presence or absence of TDP-43 aggregates in the sample or specific body part or body area.

The step of optionally correlating the presence or absence of the compound bound to the TDP-43 aggregates with the presence or absence of TDP-43 aggregates in the sample or specific body part or body area, as mentioned herein above, comprises the steps of
determining the amount of the compound bound to the TDP-43 aggregates;
correlating the amount of the compound bound to the TDP-43 aggregates with the amount of TDP-43 aggregates in the sample or specific body part or body area; and
optionally comparing the amount of the compound bound to the TDP-43 aggregates in the sample or specific body part or body area to a normal control value in a healthy control subject.

If the amount of the compound bound to the TDP-43 aggregates is higher than a normal control value of a healthy/reference subject this indicates that the patient/subject is suffering from or is at risk of developing a disease, disorder or abnormality associated with TDP-43 aggregates or a TDP-43 proteinopathy. In particular, if the amount of the compound bound to the TDP-43 aggregates is higher than what expected in a person showing no clinical evidence of neurodegenerative disease, it can be assumed that the patient has a disposition to a disease, disorder or abnormality associated with TDP-43 aggregates or to a TDP-43 proteinopathy.

### Monitoring Disease Progression:

In one embodiment, the present invention relates to a method of monitoring the progression of a disease, disorder or abnormality associated with TDP-43 aggregates or a TDP-43 proteinopathy in a patient. Typically, the patient is or has been undergoing treatment of the disease, disorder or abnormality associated with TDP-43 aggregates or the TDP-43 proteinopathy. In particular, the treatment can involve administration of an anti-TDP-43 medicament.

The method of collecting data for monitoring the progression of a disease, disorder or abnormality associated with TDP-43 aggregates or for monitoring the progression of a TDP-43 proteinopathy in a patient comprises the steps:
(a) Bringing a sample, a specific body part or body area suspected to contain TDP-43 aggregates into contact with the compound of the invention;
(b) Allowing the compound of the invention to bind to the TDP-43 aggregates;
(c) Detecting the compound of the invention bound to the TDP-43 aggregates;
(d) Optionally correlating the presence or absence of the compound of the invention bound to the TDP-43 aggregates with the presence or absence of TDP-43 aggregates in the sample or specific body part or body area; and
(e) Optionally repeating steps (a) to (c) and, if present, optional step (d) at least one time.

In order to monitor the progression over time of the disease, disorder or abnormality associated with TDP-43 aggregates or the TDP-43 proteinopathy, steps (a) to (c) and optional step (d) (if present) can be repeated one or more times. Preferably, the steps should be repeated until no further progression of the disease is observed in the patient.

The step of optionally correlating the presence or absence of the compound bound to the TDP-43 aggregates with the presence or absence of TDP-43 aggregates in the sample or specific body part or body area, as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound to the TDP-43 aggregates in the sample or specific body part or body area to a normal control value in a healthy control subject.

In the method of monitoring progression over time the amount of the compound of the invention bound to the TDP-43 aggregates can be optionally compared at various points of time during the treatment, for instance, before and after onset of the treatment and/or at various points of time after the onset of the treatment. A change, especially a decrease, in the amount of the compound of the invention bound to the TDP-43 aggregates may indicate that the disease is not progressing.

### Prognosing a Disease:

In one embodiment, the present invention relates to a method for prognosing a disease, disorder or abnormality associated with TDP-43 aggregates or a TDP-43 proteinopathy in a patient. Typically, the patient is or has been undergoing treatment of the disease, disorder or abnormality associated with TDP-43 aggregates or the TDP-43 proteinopathy. In particular, the treatment can involve administration of an anti-TDP-43 medicament.

The method of collecting data for prognosing a disease, disorder or abnormality associated with TDP-43 aggregates or for prognosing a TDP-43 proteinopathy in a patient comprises the steps:
(a) Bringing a sample, a specific body part or body area suspected to contain TDP-43 aggregates into contact with the compound of the invention; or a diagnostic composition according to the invention which comprises a compound according to the invention;
(b) Allowing the compound of the invention to bind to the TDP-43 aggregates;
(c) Detecting the compound of the invention bound to the TDP-43 aggregates;
(d) Optionally correlating the presence or absence of the compound of the invention bound to the TDP-43 aggregates with the presence or absence of TDP-43 aggregates in the sample or specific body part or body area; and
(e) Optionally repeating steps (a) to (c) and, if present, optional step (d) at least one time.

In order to prognose the progression over time of the disease, disorder or abnormality associated with TDP-43 aggregates or the TDP-43 proteinopathy, steps (a) to (c) and optional step (d) (if present) can be repeated one or more times. Preferably, the steps should be repeated until no further progression of the disease is observed in the patient.

The step of optionally correlating the presence or absence of the compound bound to the TDP-43 aggregates with the presence or absence of TDP-43 aggregates in the sample or specific body part or body area, as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound to the TDP-43 aggregates in the sample or specific body part or body area to a normal control value in a healthy control subject.

In the method of prognosing the progression over time the amount of the compound of the invention bound to the TDP-43 aggregates can be optionally compared at various points of time during the treatment, for instance, before and after onset of the treatment and/or at various points of time after the onset of the treatment. A change, especially a decrease, in the amount of the compound of the invention bound to the TDP-43 aggregates may indicate that the disease is not progressing.

### Predicting Responsiveness:

In one embodiment, the present invention relates to a method of predicting responsiveness of a patient suffering from a disease, disorder or abnormality associated with TDP-43 aggregates, or suffering from a TDP-43 proteinopathy to a treatment of said disease, disorder or abnormality associated with TDP-43 aggregates or TDP-43 proteinopathy.

The method can be used to predict the treatment which is most suitable for the patient. In particular, the treatment can involve administration of an anti-TDP-43 medicament.

The method for predicting responsiveness of a patient suffering from a disease, disorder or abnormality associated with TDP-43 aggregates or suffering from a TDP-43 proteinopathy to a treatment of said disease, disorder or abnormality associated with TDP-43 aggregates or TDP-43 proteinopathy can comprise the steps of:
(a) Bringing a sample, a specific body part or body area suspected to contain TDP-43 aggregates into contact with a compound of the invention; or with a diagnostic composition comprising a compound of the invention as disclosed herein;
(b) Allowing the compound of the invention to bind to the TDP-43 aggregates;
(c) Detecting the compound of the invention bound to the TDP-43 aggregates;
(d) Optionally correlating the presence or absence of the compound of the invention bound to the TDP-43 aggregates with the presence or absence of TDP-43 aggregates in the sample or specific body part or body area; and
(e) Optionally repeating steps (a) to (c) and, if present, optional step (d) at least one time.

Typically, the patient is/has been undergoing treatment of the disease, disorder or abnormality associated with TDP-43 aggregates or the TDP-43 proteinopathy. In particular, the treatment can involve administration of a medicament which is suitable for treating the disease, disorder or abnormality associated with TDP-43 aggregates or the TDP-43 proteinopathy.

The present method allows to predict the responsiveness of a patient to a certain treatment. In one embodiment, the responsiveness can be estimated, e.g., by repeating steps (a) to (c) and, if present, optional step (d) and monitoring the amount of the compound of the invention bound to the TDP-43 aggregates over a period of time during which the patient is undergoing treatment of the disease, disorder or abnormality associated with TDP-43 aggregates or of the TDP-43 proteinopathy. If the amount changes over time, the skilled practioner can deduce whether the patient is responsive to the treatment. Typically, if the amount of the compound of the invention bound to the TDP-43 aggregates decreases over time, it can be assumed that the patient is responsive to the treatment. Typically, if the amount of the compound bound to the TDP-43 aggregates is essentially constant or increases over time, it can be assumed that the patient is non-responsive to the treatment.

Alternatively, the responsiveness can be estimated by determining the amount of the compound of the invention bound to the TDP-43 aggregates. The amount of the compound bound to the TDP-43 aggregates can be compared to a control value such as a normal control value, a preclinical control value or a clinical control value. The control value may refer to the control value of healthy control subjects. Alternatively, the control value may refer to the control value of subjects known to be responsive to a certain therapy, or to the control value may refer to the control value of subjects known to be non-responsive to a certain therapy. The outcome with respect to responsiveness can either be “responsive” to a certain therapy, “non-responsive” to a certain therapy or “response undetermined” to a certain therapy. Response to the therapy may be different for the respective patients.

The step of optionally correlating the presence or absence of the compound bound to the TDP-43 aggregates with the presence or absence of TDP-43 aggregates in the sample or specific body part or body area, as mentioned herein above, comprises the steps of
Determining the amount of the compound bound to the TDP-43 aggregates;
Correlating the amount of the compound bound to the TDP-43 aggregates with the amount of TDP-43 aggregates in the sample or specific body part or body area; and
Optionally comparing the amount of the compound bound to the TDP-43 aggregates in the sample or specific body part or body area to a normal control value in a healthy control subject.

The control value can be, e.g., a normal control value, a preclinical control value and/or a clinical control value. A “healthy control subject” or “healthy subject” is a person showing no clinical evidence of neurodegenerative disease.

If in any of the above summarized methods the amount of the compound bound to the TDP-43 aggregates is higher than the normal control value, then it can be expected that the patient is suffering from or is likely to suffer from a disease, disorder or abnormality associated with TDP-43 aggregates or a TDP-43 proteinopathy.

Any of the compounds of the present invention can be used in the above summarized methods. Preferably detectably labeled compounds of the present invention, as disclosed herein, are employed in the above summarized methods.

In a fourth aspect, the present invention relates to the use of the compounds of the invention as a TDP-43 aggregates' diagnostic agent or diagnostic tool. In one embodiment, the present invention relates to the use of the compounds of the invention as an in vitro analytical reference or an in vitro screening tool. Said compounds of the invention are also useful in in vivo diagnostic methods. In such instances, the compounds of the invention may be detectably labeled compounds or contain cold isotopes.

In another embodiment, the present invention further relates to the use of the compounds of the present invention, more specifically detectably labelled compounds of the invention as defined herein, as diagnostic biomarkers enabling more efficient and precise patient selection, e.g., for longitudinal monitoring in clinical studies, or for supporting the development of novel therapeutics for treating TDP-43 proteinopathies. In another embodiment, the present invention further relates to the use of the compounds of the present invention, more specifically detectably labelled compounds of the invention as defined herein, as a TDP-43 aggregates' biomarker or a TDP-43 proteinopathy biomarker.

In another embodiment, the compounds of the invention may be employed for research use, in particular, as an analytical tool or reference molecule. Said compounds may also be used in detecting TDP-43 aggregates in vitro or in vivo. The compounds of the invention may be used to stain TDP-43 aggregates. For example, compounds of the invention may be used for histochemical detection in post mortem tissue such as brain tissue. The compounds of the invention are preferably detectably labelled compounds and may be directly or indirectly labelled as discussed herein.

### Kit of Parts

In a fifth aspect, the present invention relates further to a kit for use in one or more of the methods of the invention, wherein the kit comprises a compound of the invention as described herein. The kit typically comprises a container holding the compound of the invention and instructions for using the compound of the invention. Preferably, the kit comprises a compound of formula (I), as disclosed herein. More preferably, the compound of the invention is a detectably labelled compound (e.g. compound of formula (I-T) or subformula (I-Ta) or (I-Tb), or (I-F) or subformula (I-Fa) or (I-Fb)).

The term “kit” refers in general to any diagnostic kit known in the art. More specifically, the latter term refers to a diagnostic kit as described in Zrein et al., Clin. Diagn. Lab. Immunol., 1998, 5, 45-49.

The dose of the detectably labelled compounds of the present invention will vary depending on the exact compound to be administered, the weight of the patient, size and type of the sample, and other variables as would be apparent to a physician skilled in the art. Generally, the dose could preferably lie in the range 0.001 μg/kg to 10 μg/kg, preferably 0.01 μg/kg to 1.0 μg/kg. The radioactive dose can be, e.g., 100 to 600 MBq, more preferably 150 to 450 MBq.

In particular, such kits may be useful for performing the methods of the invention (which include, for example, but not limited to, imaging, diagnosing, and monitoring methods), e.g., for diagnosing of a disease, disorder or abnormality associated with TDP-43 aggregates or a TDP-43 proteinopathy. Such kits may comprise all necessary components for performing the herein provided methods. Typically, each component is stored separately in a single overall packaging. Suitable additional components for inclusion in the kits are, for example, buffers, detectable dyes, laboratory equipment, reaction containers, instructions and the like. Instructions for use may be tailored to the specific method for which the kit is to be employed.

The present invention relates further to a kit for the preparation of a detectably labeled compound of the invention, wherein in particular the detectable label is a radioisotope. Thus, the kit comprises a precursor of the detectably labeled compound of the formula (I) and a labeling agent which reacts with the precursor to introduce a detectable (e.g., radioactive) label. Preferred precursors are compounds of the formulae (II), (III) or (IV). The labeling agent which reacts with the precursor can be an agent which introduces a detectable (e.g., radioactive) label such as ¹⁸F or ³H. The labeling agent can be a ¹⁸F-fluorination agent.

The present invention relates further to a kit for preparing a radiopharmaceutical preparation comprising a precursor of the detectably labeled compound of the formula (I), wherein the precursor is a compound of formula (II), or a compound of formula (III), or a compound of formula (IV).

### Method for Preparing a Compound of the Invention

In a sixth aspect, the present invention relates further to a method for preparing a compound of formula (I).

### Cold Isotope Compounds:

In one embodiment, the present invention relates to a method for preparing a compound of formula (Ia), as described above, the method comprising the step of:

Reacting a compound of formula (II) with R¹³ to provide a compound of formula (Ia)

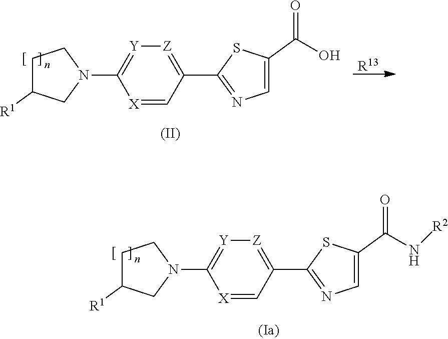

wherein
n, R¹, R², and X, Y and Z are as defined above;
R¹³ is a 5- or 6-membered carbocyclic or heterocyclic compound which is substituted with NH₂ and which can be optionally further substituted with F, NH₂, CN and/or CH₃, wherein the heterocyclic ring contains one or more heteroatoms selected from N, O and/or S.

The compound having the formula (II), or a stereoisomer, polymorph, racemic mixture, tautomer, pharmaceutically acceptable salt, hydrate, or solvate thereof, or mixtures thereof, is an aspect of the present invention:

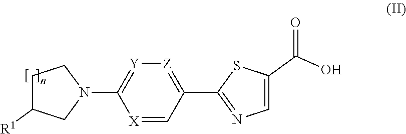

wherein
n is 1 or 2;
X, Y and Z are independently CH or N; and
R¹ is H or F.

In a preferred embodiment, R¹³ is selected from the following compounds:

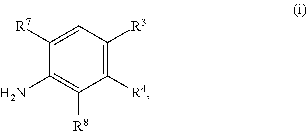

wherein

| | | | |
|---|---|---|---|
| R³ is F, | R⁴ is NH₂, | R⁷ is H, and | R⁸ is H (preferred embodiment); or |
| R³ is NH₂, | R⁴ is F, | R⁷ is H, and | R⁸ is H; or |
| R³ is CN, | R⁴ is NH₂, | R⁷ is H, and | R⁸ is H; or |
| R³ is H, | R⁴ is NH₂, | R⁷ is H, and | R⁸ is CN; or |
| R³ is H, | R⁴ is NH₂, | R⁷ is H, and | R⁸ is F; |

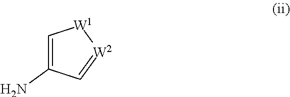

wherein W¹ is S and W² is N;

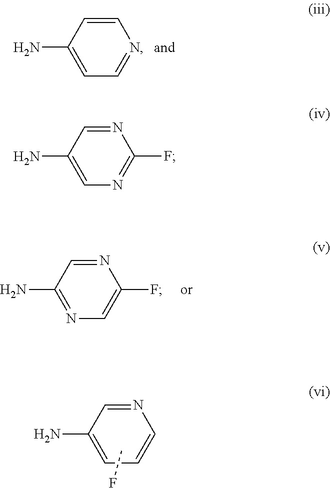

wherein

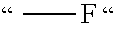

indicates that “F” may be present or absent.

The method of reacting the compound having the formula (II) with R¹³ can be conducted by any suitable method. In one option, the reaction can be conducted in the presence of a diamine chelator such as DMEDA, a base such as potassium carbonate, a catalyst such as CuI, and an aprotic solvent such as dioxane. In another option, the reaction can be conducted under Pd-coupling conditions, in the presence of a Pd catalyst such as Pd[P(Ph)₃]₄ or Pd(OAc)₂, XantPhos.

In another embodiment, the present invention relates to a method for preparing a compound of formula (Ib), as described above, the method comprising the step of:

Reacting a compound of formula (II′) with R¹⁴ to provide a compound of formula (I)

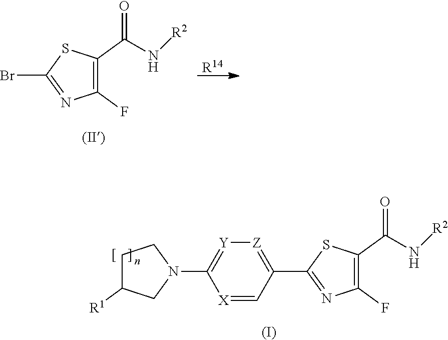

wherein
R¹⁴ is

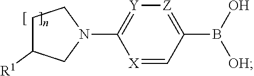

and
n, R¹, R², and X, Y and Z are as defined for formula (I) above.

The compound having the formula (II′), or a stereoisomer, polymorph, racemic mixture, tautomer, pharmaceutically acceptable salt, hydrate, or solvate thereof, or mixtures thereof, is an aspect of the present invention:

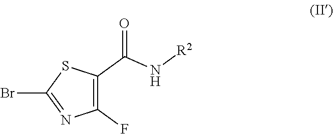

wherein
R² is as defined above.

In a preferred embodiment R¹⁴ is

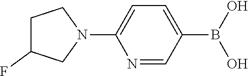 [SMILES: OB(O)c1ccc(N2CCC(F)C2)nc1]

### Tritium (³H) Detectably Labeled Compounds

In one embodiment, the present invention provides a method for preparing the Tritium (³H) detectably labeled compounds of the invention having the formula (I-T), said method comprises the step of radiolabeling a precursor of the compound having the formula (I-T) with a radioisotope, wherein at least one leaving group of the precursor of the compound having the formula (I-T) is replaced by Tritium (³H).

Tritium (³H) detectably labeled compounds having the formula (I-T) are preferably defined wherein at least 1 to 3 Hydrogen (H) are each replaced by Tritium (³H). Tritium (³H) detectably labeled compounds having the formula (I-T) are more preferably defined wherein 1 or 2 Hydrogen (H) are replaced by Tritium (³H).

In another embodiment, the present invention provides a compound of formula (III)

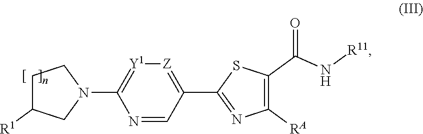

or a stereoisomer, polymorph, racemic mixture, tautomer, pharmaceutically acceptable salt, hydrate, or solvate thereof, or mixtures thereof,
wherein
n is 1 or 2;
R¹ is H or F;
Y¹ is selected from C—Br, C—I, and C—H,
Z is selected from CH or N,
R^{A} is H or F,
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and at least one of Y¹ and/or R¹¹ comprises Br or I.

In one embodiment, the present invention provides a compound of formula (III) having the subformula (IIIa):

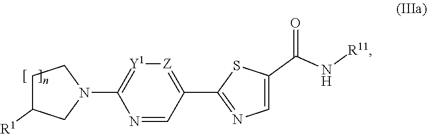

or a stereoisomer, polymorph, racemic mixture, tautomer, pharmaceutically acceptable salt, hydrate, or solvate thereof, or mixtures thereof,
wherein
n is 1 or 2;
R¹ is H or F;
Y¹ is selected from C—Br, C—I, and C—H,
Z is selected from CH or N,
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and at least one of Y¹ and/or R¹¹ comprises Br or I.

In another embodiment, the present invention provides a compound of formula (III) having the subformula (IIIb):

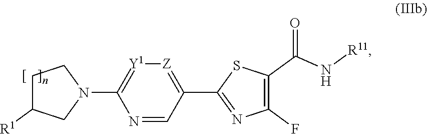

or a stereoisomer, polymorph, racemic mixture, tautomer, pharmaceutically acceptable salt, hydrate, or solvate thereof, or mixtures thereof,
wherein
n is 1 or 2;
R¹ is H or F;
Y¹ is selected from C—Br, C-A, and C—H,
Z is selected from CH or N,
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and at least one of Y¹ and/or R¹¹ comprises Br or 1.

In a preferred embodiment, Y¹ is C—Br or C—I and the Br or I is replaced by T.

In another preferred embodiment, R¹¹ comprises Br or I and the Br or I is replaced by T.

In one preferred embodiment Y¹ is C—Br or C—I and the Br or I is replaced by T; and R¹¹ comprises Br or I and the Br or I is replaced by T.

Preferably, the Tritium (³H) detectably labeled compounds having the formula (I-T) according to the present invention include (wherein T means ³H)

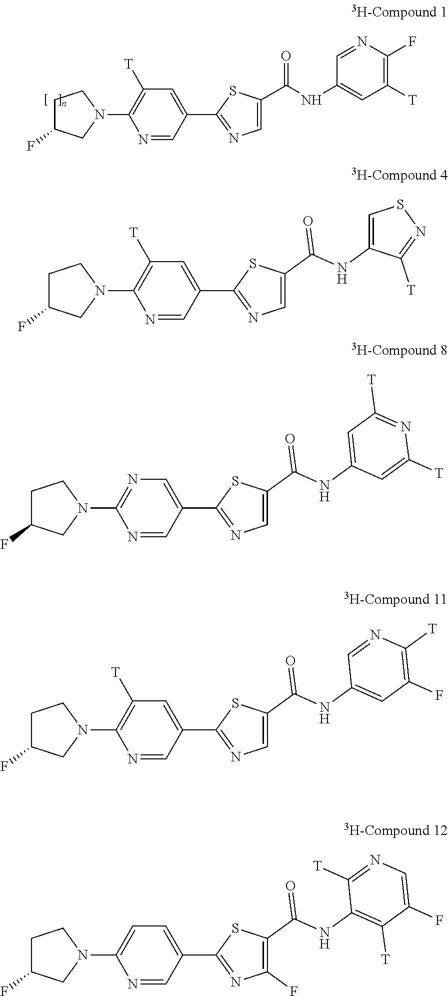

In another embodiment, the present invention provides a method for preparing a compound of formula (I-T), said method comprises the step of:

Radiolabeling a precursor compound having the formula (III) with T (i.e., ³H) by either exchange of Br or I with T using T₂ (preferably using a catalyst, for example Pd/C catalyst) or introduction of a CT₃-group

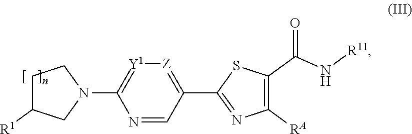

or a stereoisomer, polymorph, racemic mixture, tautomer, pharmaceutically acceptable salt, hydrate, or solvate thereof, or mixtures thereof,
wherein
n is 1 or 2;
R¹ is H or F;
R^{A} is H or F;
Y¹ is selected from C—Br, C—I, and C—H,
Z is selected from CH or N,
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and either at least one of Y¹ and/or R¹¹ comprises Br or I.
wherein the at least one Br or I is replaced by T; or
R¹¹ comprises a 5- or 6-membered carbocyclic or heterocyclic ring containing a NH moiety;
wherein the NH-moiety is substituted by N-CT₃,
and wherein T is ³H.

Preferably, the precursors having the formula (III) according to the present invention can be selected from

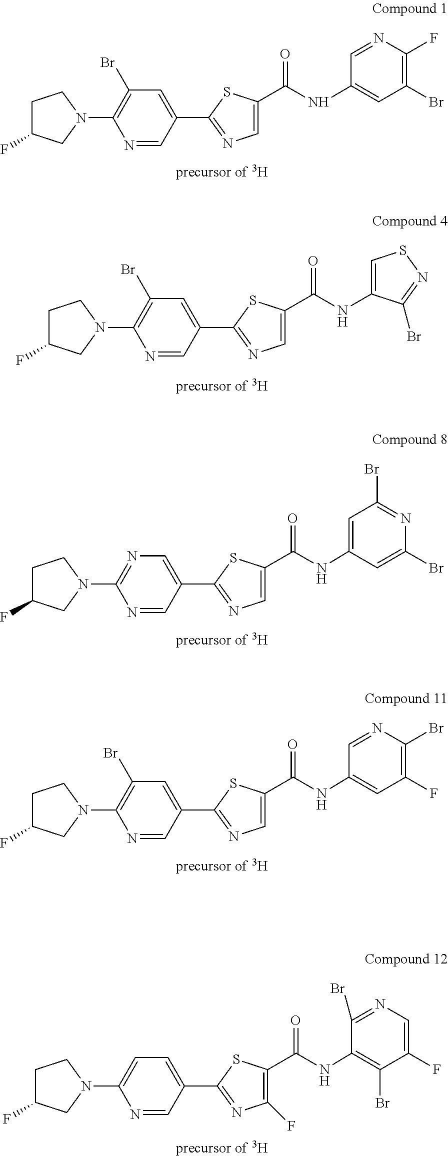 [SMILES: O=C(Nc1cnc(F)c(Br)c1)c1cnc(-c2cnc(N3CC[C@@H](F)C3)c(Br)c2)s1 ; O=C(Nc1csnc1Br)c1cnc(-c2cnc(N3CC[C@@H](F)C3)c(Br)c2)s1 ; O=C(Nc1cc(Br)nc(Br)c1)c1cnc(-c2cnc(N3CC[C@H](F)C3)nc2)s1 ; O=C(Nc1cnc(Br)c(F)c1)c1cnc(-c2cnc(N3CC[C@@H](F)C3)c(Br)c2)s1 ; O=C(Nc1c(Br)ncc(F)c1Br)c1sc(-c2ccc(N3CC[C@@H](F)C3)nc2)nc1F]

The methods used for introducing a radioisotope such as ³H are well known in the art and include the methods described below.

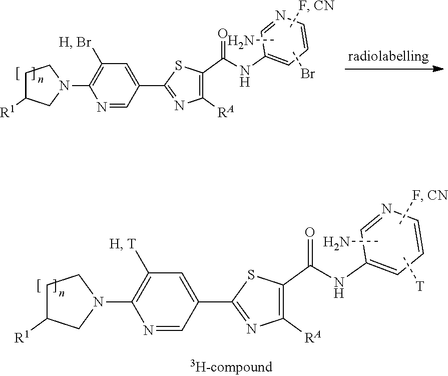

In this scheme, the substituents Br, NH₂, F and CN are just shown as an example. The definitions of formula (I-T) apply in this respect. A further example is shown in the following scheme:

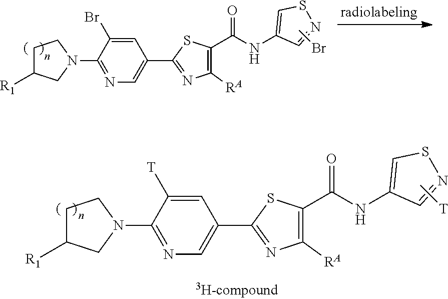

For the introduction of T the ³H radiolabeling agent can be tritium gas. The method can be conducted in the presence of a catalyst such as palladium on carbon (Pd/C) or Lindlar's catalyst, a solvent such as N,N-dimethylformamide (DMF) and a base such as N,N-diisopropylethylamine (DIPEA).

In one embodiment, the present invention relates to a method for preparing a precursor compound of formula (III), as described above, the method comprising the step of: Reacting a compound of formula (II) as defined above with R¹³ to provide a compound of formula (Ia) followed by either NBS bromination or acid cleavage of a trimethylsilylethoxymethyl (SEM)-protecting group. The following example is given as an illustration:

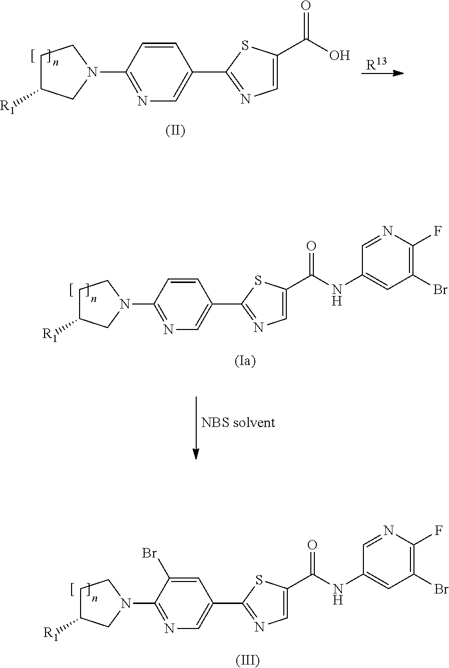

wherein
n, R¹ and R¹³ are as defined above.

Compounds with R¹³ having a different ring structure can be prepared in a similar way.

In a preferred embodiment, R¹³ is selected from the following compounds:

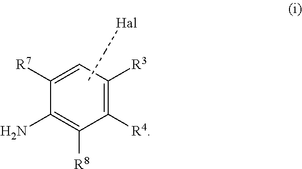

wherein Hal is Br or I; and

| | | | |
|---|---|---|---|
| R³ is F, | R⁴ is NH₂, | R⁷ is H, and | R⁸ is H (preferred embodiment); or |
| R³ is NH₂, | R⁴ is F, | R⁷ is H, and | R⁸ is H; or |
| R³ is CN, | R⁴ is NH₂, | R⁷ is H, and | R⁸ is H; or |
| R³ is H, | R⁴ is NH₂, | R⁷ is H, and | R⁸ is CN; or |
| R³ is H, | R⁴ is NH₂, | R⁷ is H, and | R⁸ is F; |

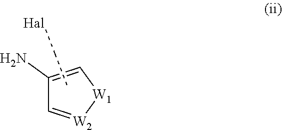

wherein W¹ is S and W² is N; and wherein Hal is Br or I;

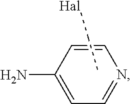

wherein Hal is Br or I; and

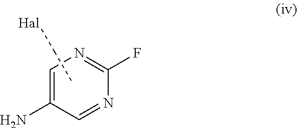

wherein Hal is Br;

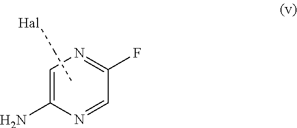

wherein Hal is Br; or

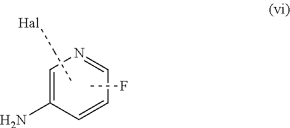

wherein Hal is Br.

In all of the above

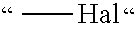

indicates that “Hal” may be present or absent.

The method of reacting the compound having the formula (II) with R¹³ can be conducted by any suitable method. In one option, the reaction can be conducted in the presence of a diamine chelator such as DMEDA, a base such as potassium carbonate, a catalyst such as CuI, and an aprotic solvent such as dioxane. In another option, the reaction can be conducted under Pd-coupling conditions, in the presence of a Pd catalyst such as Pd[P(Ph)₃]₄ or Pd(OAc)₂, XantPhos.

### Fluorine (¹⁸F) Detectably Labeled Compounds:

In one embodiment, the present invention provides a method for preparing the Fluorine (¹⁸F) detectably labeled compounds of the invention, said method comprises radiolabeling a precursor having the formula (IV) with a radioisotope [¹⁸F]:

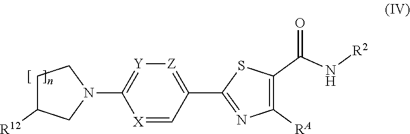

or a stereoisomer, polymorph, racemic mixture, tautomer, pharmaceutically acceptable salt, hydrate, or solvate thereof, or mixtures thereof,
wherein
n, R^{A}, R², and X, Y and Z are as defined for formula (I) above;
R¹² is a leaving group (LG), preferably a mesylate group, which is suitable for being replaced by ¹⁸F in the radiolabeling step.

In one embodiment, the present invention provides a method for preparing the Fluorine (¹⁸F) detectably labeled compounds of the invention, said method comprises radiolabeling a precursor of formula (IV) having the formula (IVa) with a radioisotope [¹⁸F]:

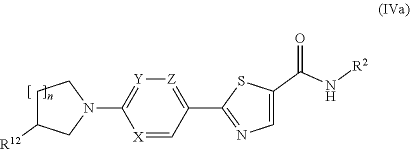

or a stereoisomer, polymorph, racemic mixture, tautomer, pharmaceutically acceptable salt, hydrate, or solvate thereof, or mixtures thereof,
wherein
n is 1 or 2;
R² is

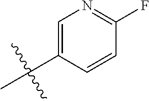 [SMILES: *c1ccc(F)nc1]

R¹² is a leaving group (LG), preferably a mesylate group, which is suitable for being replaced by ¹⁸F in the radiolabeling step.

Compounds with R² having a different ring structures as defined above can be prepared in a similar way.

The fluorination can be conducted in the presence of a ¹⁸F-fluorination agent which can be selected from K[¹⁸F], Cs¹⁸F, Na¹⁸F, Rb¹⁸F, Kryptofix[222]K¹⁸F, tetra(C₁₋₆alkyl) ammonium salt of ¹⁸F, and tetrabutylammonium [¹⁸F]fluoride.

Preferably, the Leaving Group (LG) is C₁₋₄ alkyl sulfonate or C₆₋₁₀ aryl sulfonate or nitro. More preferably, the Leaving Group (LG) is mesylate, tosylate or nosylate or nitro. Even more preferably, the Leaving Group (LG) is mesylate or nitro.

Suitable solvents for the ¹⁸F-fluorination step are known to a skilled person. The solvent can be, for example, selected from the group consisting of DMF, DMSO, acetonitrile, DMA, or mixtures thereof. Preferably, the solvent is acetonitrile or DMSO.

Preferably, the method for preparing the Fluorine (¹⁸F) detectably labeled Compound 1 comprises a radiolabeling step in which the Leaving Group (LG), which in this case is mesylate, of the precursor L1 is replaced with a Fluorine (¹⁸F) in the presence of the ¹⁸F-fluorinating agent, such as K[¹⁸F] or [¹⁸F]TBAF, as shown below:

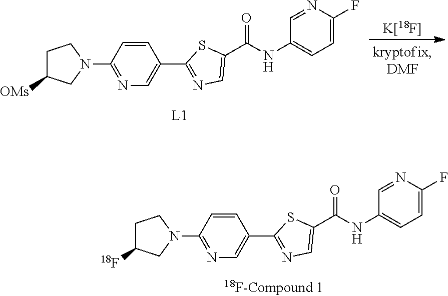

The compounds of the invention can be prepared by one of the general methods shown in the following schemes. These methods are only given for illustrative purposes and should not be construed as limiting.

The precursor compounds having the formulae (II), (III), or (IV) as defined above or the stereoisomer, the polymorph, the racemic mixture, the tautomer, the pharmaceutically acceptable salt, the hydrate, or the solvate thereof are part of the invention.

### Abbreviations

CH₃CN Acetonitrile
CCl₄ Carbon tetrachloride
Cs₂CO₃ Cesium carbonate
CuBr₂ Copper bromide
DCM Dichloromethane
DIPEA N,N-Diisopropylethylamine
DMAP Dimethylaminopyridine
DMEDA 1,2-Dimethylethylenediamine
DMF Dimethylformamide
DMSO Dimethylsulfoxide
EtOH Ethanol
EtOAc Ethyl Acetate
h Hour
HATU Hexafluorophosphate Azabenzotriazole Tetramethyl Uronium
HCl Hydrochloric acid
HPLC High performance liquid chromatography
K₂CO₃ Potassium carbonate
n-BuOH n-butanol
LCMS Liquid chromatography-mass spectrometry
LiOH Lithium hydroxide
MW Microwaves
MeSO₂Cl Methanesulfonyl chloride
MeOH Methanol
NMR Nuclear magnetic resonance
NBS N-bromosuccinimide
Pd(PPh₃)₄ Palladium tetrakistriphenylphosphine
Pd(dppf)Cl₂ 1,1′-Bis(diphenylphosphino)ferrocene dichloropalladium (II)
RT Room Temperature (approx. 25° C.)
TBDMSCl Tert-butyldimethylsilylchloride
TEA Triethylamine
TFA Trifluoroacetic acid
THF Tetrahydrofuran
^{t}BuONO Tert-butyl nitrite
XPhos Pd G₂ Chloro(2-dicyclohexylphosphino-2′,4′,6′-triisopropyl-1,1′-biphenyl)[2-(2′-amino-1,1-biphenyl)]palladium(II)

### General Synthetic Schemes:

### Synthetic Scheme for the Preparation (R)—N-(6-fluoropyridin-3-yl)-2-(6-(3-fluoropyrrolidin-1-yl)pyridin-3-yl)thiazole-5-carboxamide (Compound 1)

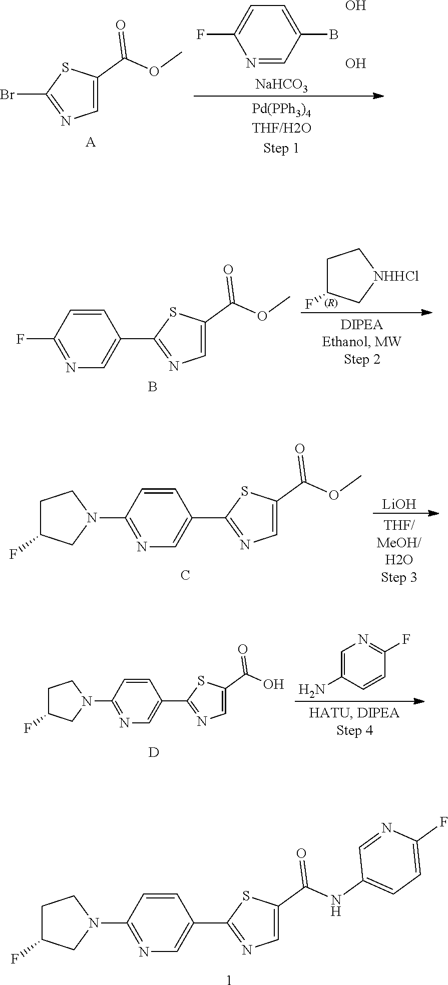

### Synthetic Scheme for the Preparation (R)-4-fluoro-N-(6-fluoropyridin-3-yl)-2-(6-(3-fluoropyrrolidin-1-yl)pyridin-3-yl)thiazole-5-carboxamide (Compound 13)

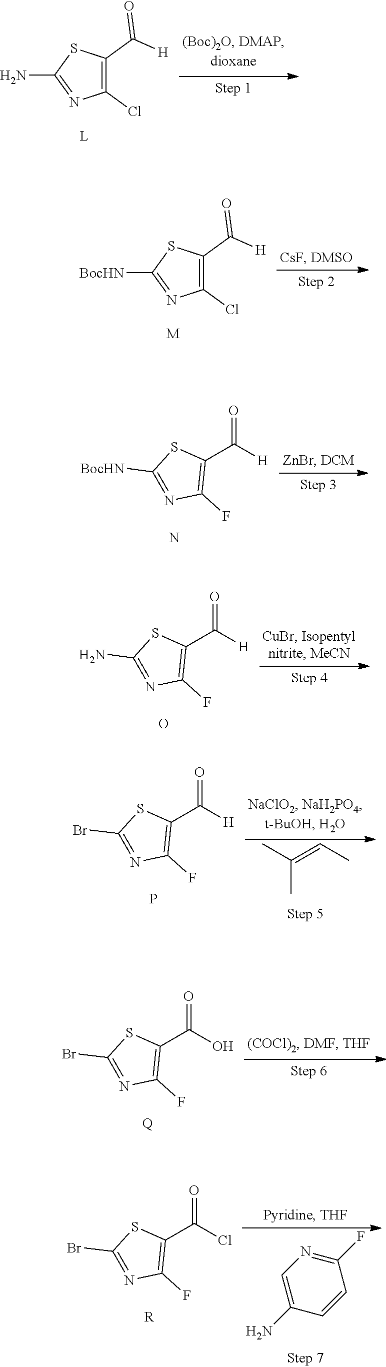

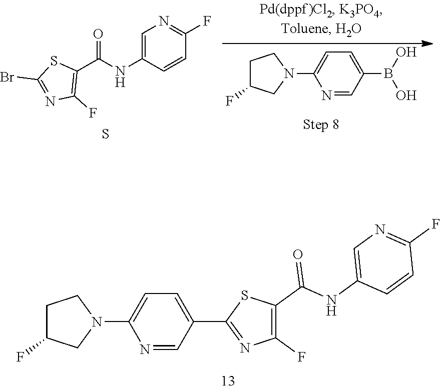

### Synthetic Scheme for the Preparation (R)-4-fluoro-N-(5-fluoropyridin-3-yl)-2-(6-(3-fluoropyrrolidin-1-yl)pyridin-3-yl)thiazole-5-carboxamide (Compound 12)

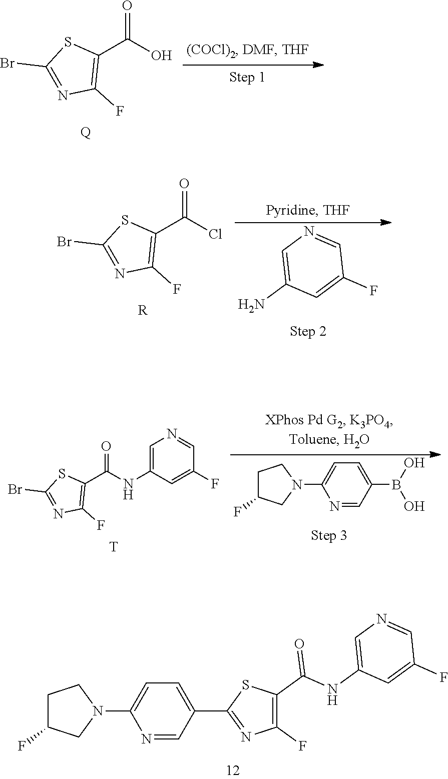

### Synthetic Scheme for the Preparation of [³H] Precursor Compounds

### Synthetic Scheme for the Preparation of (R)-2-(5-bromo-6-(3-fluoropyrrolidin-1-yl)pyridin-3-yl)-N-(5-bromo-6-fluoropyridin-3-yl)thiazole-5-carboxamide (precursor of ³H Compound 1) (Compound 2)

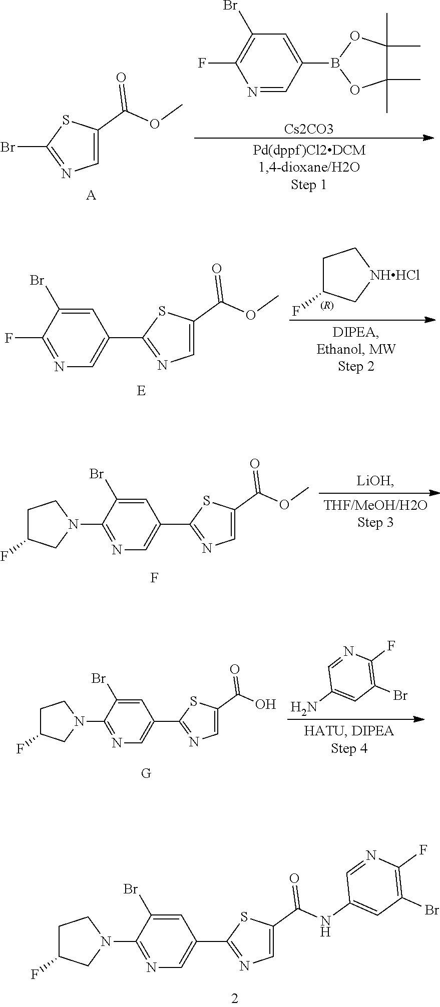

### Synthetic Scheme for the Preparation of (R)—N-(2,4-dibromo-5-fluoropyridin-3-yl)-4-fluoro-2-(6-(3-fluoropyrrolidin-1-yl)pyridin-3-yl)thiazole-5-carboxamide (precursor of ³H Compound 12) (Compound 20)

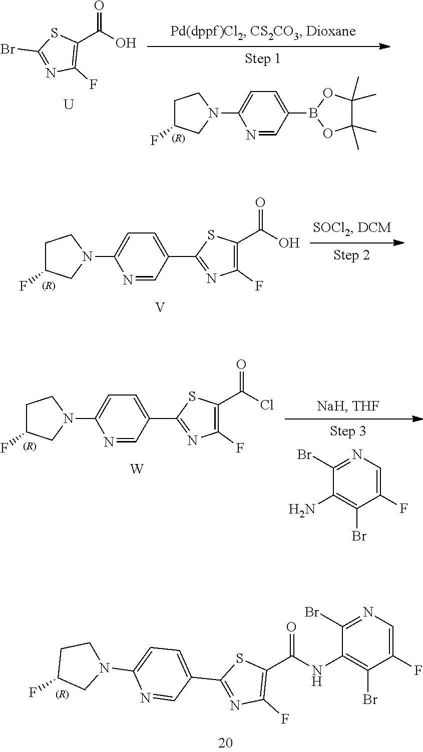

³H labeled compounds can be prepared from a suitable precursor compound containing halogen atoms by catalytic tritiodehalogenation with tritium gas (M. Saljoughian Synthesis (2002), 1781-1801), or from a suitable precursor compound containing a NH moiety by methylation with methyl iodide [³H](Y. Chen, Chemistry 25 (2019):3405-3439). Preferably, the solvents used in the ³H-labeling are DMF or DMA, preferably the solvent is DMF.

### Synthesis Scheme for the Preparation of ³H Labelling

### Synthesis of tritiated 2-[2-fluoro-6-[(3R)-3-fluoropyrrolidin-1-yl]-3-pyridyl]-5-(3-pyridyl)-6,7-dihydrothiazolo[5,4-c]pyridin-4-one ³H-Compound 1

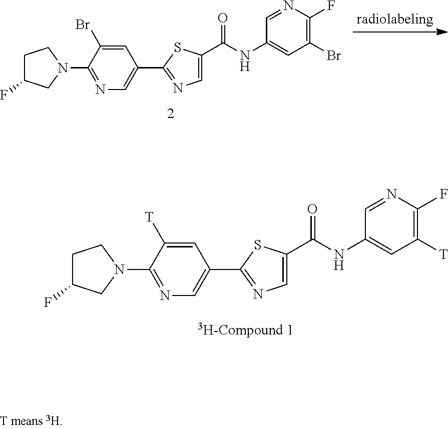

### Synthetic Schemes for the Preparation of Precursors for ¹⁸F-Labeling

### Synthetic Scheme for the Preparation of (S)-1-(5-(5-((6-fluoropyridin-3-yl)carbamoyl)thiazol-2-yl)pyridin-2-yl)pyrrolidin-3-yl methanesulfonate (precursor of ¹⁸F Compound 1) (Compound 3)

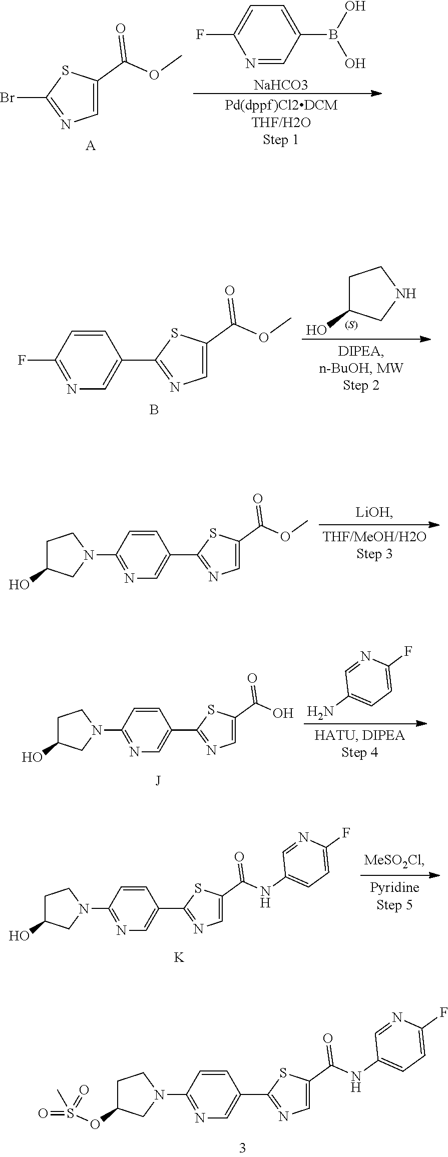

The reactions take place in the presence of a fluorinating agent and typically a solvent.

¹⁸F labeled compounds can be prepared by reacting the precursor compounds containing a LG with an ¹⁸F-fluorinating agent, so that the LG is replaced by ¹⁸F. The ¹⁸F-fluorinating agent can be a tetraalkylammonium salt of ¹⁸F (such as tetra(C₁₋₆ alkyl)ammonium salt of ¹⁸F, e.g., tetrabutylammonium [¹⁸F]fluoride), a tetraalkylphosphonium salt of ¹⁸F (such as tetra(C₁₋₈ alkyl) phosphonium salt of ¹⁸F), K[¹⁸F], Cs¹⁸F, Na¹⁸F, Rb¹⁸F, or Kryptofix[222]K¹⁸F. Preferably, the ¹⁸F-fluorination agent is Cs¹⁸F, K¹⁸F, or tetrabutylammonium [⁸F]fluoride. The reagents, solvents and conditions which can be used for the ¹⁸F-fluorination are well-known to a skilled person in the field (L. Cai, S. Lu, V. Pike, Eur. J. Org. Chem. 2008, 2853-2873; J. Fluorine Chem., 27 (1985):177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50). Preferably, the solvents used in the ¹⁸F-fluorination are DMF, DMSO, acetonitrile, DMA, or mixtures thereof, preferably the solvent is acetonitrile or DMSO.

Although the reaction is shown above with respect to ¹⁸F as a radioactive label, other radioactive labels can be introduced following similar procedures.

The invention is illustrated by the following examples which, however, should not be construed as limiting.

### EXAMPLES

All reagents and solvents were obtained from commercial sources and were used without further purification. Proton (¹H) NMR spectra were recorded on a Bruker DRX-400 MHz NMR spectrometer or on a Bruker AV-400 MHz NMR spectrometer in deuterated solvents. Mass spectra (MS) were recorded on an Advion CMS mass spectrometer. Chromatography was performed using silica gel (Fluka: Silica gel 60, 0.063-0.2 mm) and suitable solvents as indicated in the specific examples. Flash purification was conducted with a Biotage Isolera One flash purification system using HP-Sil or KP-NH SNAP cartridges (Biotage) and the solvent gradient indicated in the specific examples. Thin layer chromatography (TLC) was carried out on silica gel plates with UV detection.

Although some of the present examples do not indicate that the respective compounds were detectably labeled, it is understood that corresponding detectably labeled compounds are intended and can be easily prepared, e.g., by using detectably labeled starting materials, such as starting materials containing C(³H)₃, (¹¹C)H₃ or ¹⁸F.

### Example 1 Synthesis of (R)—N-(6-fluoropyridin-3-yl)-2-(6-(3-fluoropyrrolidin-1-yl)pyridin-3-yl)thiazole-5-carboxamide Compound 1

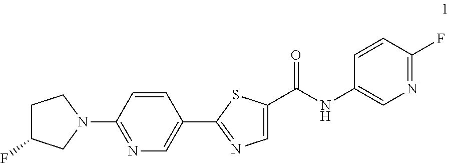 [SMILES: O=C(Nc1ccc(F)nc1)c1cnc(-c2ccc(N3CC[C@@H](F)C3)nc2)s1]

### Step-1: methyl 2-(6-fluoropyridin-3-yl) thiazole-5-carboxylate (B)

In an oven-dried round bottom flask was added methyl 2-bromothiazole-5-carboxylate (3.0 g, 13 mmol), boronic acid (2.8 g, 20 mmol), NaHCO₃ (5.6 g, 65 mmol), and (THF:H₂O) (1:1, 45 mL, 15 vol) under an argon atmosphere. The reaction mixture was degassed with argon for 15 min. Then Pd(PPh₃)₄ (1.5 g, 0.13 mmol) was added, and the mixture was heated to 90° C. for 12 h. The reactants were consumed as monitored by TLC. The reaction mixture was quenched with ice-water (20 mL) and extracted in EtOAc (70 mL×3). The organic layer was dried over Na₂SO₄, concentrated and purified by silica gel chromatography (230-400 mesh) and eluted in 20% EtOAc in hexane to get (B) as a brownish liquid (2.0 g, 62%). MS (ESI): 239.1 [M]⁺. 1H NMR (CDCl₃) δ 8.71 (s, 1H), 8.61 (m, 1H), 8.46 (s, 1H), 7.07 (dd, 1H), 3.95 (s, 3H).

### Step-2: methyl (R)-2-(6-(3-fluoropyrrolidin-1-yl) pyridin-3-yl) thiazole-5-carboxylate (C)

Compound (B) (2.0 g, 8.4 mmol), (R)-3-fluoropyrrolidine hydrogen chloride (1.37 g, 11 mmol), DIPEA (3.0 mL, 16.8 mmol), and NMP (20 mL, 10 vol) were taken in an oven-dried microwave vial under argon atmosphere. The reaction mixture was irradiated at 120° C. for 2 h. After completion, the reaction mixture was quenched with ice cold water (10 mL), extracted with EtOAc (50 mL×3). Combined organic layers were washed with cold brine solution (20 mL×2), dried over Na₂SO₄ and concentrated under vacuum. The obtained crude mass was purified by column chromatography over silica gel (100-200 mesh) and eluted in 20% EtOAc in hexane to afford (C) as a white solid (1.9 g, 76%). MS (ESI): 308.17 [M+H]⁺; 1H NMR (CDCl₃) δ 8.78 (m, 1H), 8.35 (s, 1H), 8.05 (dd, 1H), 6.45 (d, 1H), 5.41 (dt, 1H), 3.92 (s, 3H), 3.70 (m, 3H), 2.44 (m, 1H), 2.19 (m, 3H), 1.51 (m, 1H).

### Step-3: (R)-2-(6-(3-fluoropyrrolidin-1-yl) pyridin-3-yl) thiazole-5-carboxylic acid (D)

To a solution of (C) (1.5 g, 4.5 mmol) in (THF:MeOH:water) (2:2:1, 75 mL, 50 vol) was added lithium hydroxide (233 mg, 9.7 mmol) and the mixture was kept at RT for 3 h. Then the mixture was treated with 2M HCl (aq) solution until the pH reached up to 2-3. The biphasic mixture was filtered through a Büchner funnel. The obtained mass was washed with hexane (5 mL×3), dried under high vacuum to afford (D) as an off-white solid (1.4 g, 78%). MS (ESI): 294.15 [M+H]⁺; 1H NMR (DMSO-D6) δ 13.49 (s, 1H), 8.75 (d, 1H), 8.29 (s, 1H), 8.07 (dd, 1H), 6.63 (d, 1H), 5.54 (s, 1H), 3.73 (m, 3H), 3.49 (m, 1H), 2.23 (m, 2H).

### Step-4: (R)—N-(6-fluoropyridin-3-yl)-2-(6-(3-fluoropyrrolidin-1-yl) pyridin-3-yl) thiazole-5-carboxamide (1)

To a solution of (D) (150 mg, 0.5 mmol) in DMF (3.0 mL, 20 vol) was added HATU (380 mg, 1.0 mmol) followed by DIPEA (0.26 mL, 1.5 mmol) at 0° C. The resulting mixture was stirred at RT for 20 min. The reaction mixture was again cooled to 0° C. and to this was added 6-fluoropyridin-3-amine (68 mg, 0.6 mmol). The resulting mixture was stirred at RT for 16 h. After completion, the reaction mixture was quenched with ice-cool water (3.0 mL). A solid precipitate was formed. The crude reaction mass was filtered through a Büchner funnel. The obtained mass was washed with hexane (3 mL×3), and dried under high vacuum to afford (1) as an off-white solid (120 mg, 62%) LCMS (ESI): 387.8 [M]⁺; 1H NMR (DMSO-D6) δ 10.68 (s, 1H), 8.77 (d, 1H), 8.58 (s, 1H), 8.53 (s, 1H), 8.27 (m, 1H), 8.10 (dd, 1H), 7.24 (dd, 1H), 6.65 (d, 1H), 5.48 (d, 1H), 3.74 (m, 3H), 3.48 (m, 1H), 2.24 (m, 2H).

### Example 2 Synthesis of 5-(3-amino-2,6-dibromo-4-fluorophenyl)-2-(5-bromo-6-(pyrrolidin-1-yl)pyridin-3-yl)-6,7-dihydrothiazolo[5,4-c]pyridin-4(5H)-one Compound 2

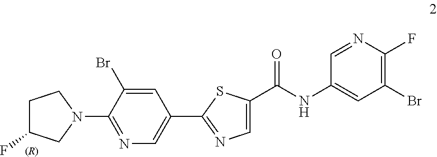

### Step-1: methyl 2-(5-bromo-6-fluoropyridin-3-yl) thiazole-5-carboxylate (E)

In an oven-dried round bottom flask was added (A) (0.6 g, 2.7 mmol), boronic ester (0.9 g, 2.9 mmol), Cs₂CO₃ (1.75 g, 5.4 mmol), and (1,4-dioxane:H₂O) (4:1, 30 mL, 50 vol) under an argon atmosphere. The reaction mixture was degassed with argon for 15 min. Then Pd(dppf)Cl₂·DCM (0.22 g, 0.27 mmol) was added and the mixture was heated to 90° C. for 3 h. The reactants were consumed as monitored by TLC. The reaction mixture was then quenched with ice-water (10 mL) and extracted in DCM (50 mL×3). The organic layer was dried over Na₂SO₄, concentrated under vacuum and purified by silica gel chromatography (100-200 mesh) and eluted in 10% EtOAc in hexane to afford (E) as a brownish liquid (120 mg, 14%). MS (ESI): 317.09 [M]*. 1H NMR (CDCl₃) δ 8.71 (s, 1H), 8.61 (m, 1H), 8.46 (s, 1H), 3.96 (s, 3H), 3.90 (s, 1H).

### Step-2: methyl (R)-2-(5-bromo-6-(3-fluoropyrrolidin-1-yl) pyridin-3-yl) thiazole-5-carboxylate (F)

Compound (E) (120 mg, 0.38 mmol), (R)-3-fluoropyrrolidine hydrogen chloride (72 mg, 0.56 mmol), DIPEA (0.2 mL, 1.1 mmol), and EtOH (2.0 mL, 17 vol) were taken in an oven-dried microwave vial under argon atmosphere. The reaction mixture was irradiated at 120° C. for 2 h. After completion, the reaction mixture was quenched with ice cold water (4 mL) and extracted with EtOAc (10 mL×3). The combined organic layers were washed with cold brine solution (10 mL), dried over Na₂SO₄ and concentrated under vacuum. The obtained crude mass was purified by column chromatography over silica gel (100-200 mesh) and eluted in 20% EtOAc in hexane to obtain (F) as a white solid (100 mg, 69%). MS (ESI): 387.96 [M+H]⁺; 1H NMR (CDCl₃) δ 8.65 (d, 1H), 8.36 (d, 1H), 8.33 (d, 1H), 5.34 (d, 1H), 4.06 (m, 1H), 3.92 (s, 3H), 2.37 (m, 1H), 2.07 (m, 1H).

### Step-3: (R)-2-(5-bromo-6-(3-fluoropyrrolidin-1-yl) pyridin-3-yl) thiazole-5-carboxylic acid (G)

To a solution of (F) (120 mg, 0.3 mmol) in (THF:MeOH:water) (2:2:1, 6.0 mL, 50 vol) was added lithium hydroxide monohydrate (15 mg, 0.6 mmol) and the mixture was kept at RT for 2 h. Then the mixture was treated with 2M HCl (aq) solution until the pH reached up to 2-3. The biphasic mixture was stirred for 5 min and the layers were separated. The aqueous layer was further extracted with 10% MeOH in DCM (2×20 mL) and the combined organic layers were dried over Na₂SO₄ and concentrated under vacuum to get (G) as a yellowish solid (100 mg, 86%). MS (ESI): 372.96 [M+H]⁺; 1H NMR (DMSO-D6) δ 13.52 (s, 1H), 8.74 (d, 1H), 8.35 (m, 2H), 5.43 (d, 1H), 4.06 (dd, 1H), 3.89 (m, 4H), 2.17 (m, 2H).

### Step-4: (R)-2-(5-bromo-6-(3-fluoropyrrolidin-1-yl) pyridin-3-yl)-N-(5-bromo-6-fluoropyridin-3-yl) thiazole-5-carboxamide (2)

To a solution of (G) (50 mg, 0.13 mmol) in DCM (2.5 mL, 50 vol) was added HATU (67 mg, 0.17 mmol) followed by DIPEA (0.07 mL, 0.4 mmol) at 0° C. The resulting mixture was stirred at RT for 20 min. The reaction mixture was again cooled to 0° C. and to this was added 5-bromo-6-fluoropyridin-3-amine (33 mg, 0.17 mmol). The resulting mixture was stirred at RT for 16 h. After completion, the reaction mixture was quenched with ice-cool water and the product was extracted with 10% MeOH in DCM (20 mL×3). The extract was dried over Na₂SO₄ and concentrated under vacuum. The residue was purified by column chromatography over silica gel (230-400 mesh) and eluted in 3% MeOH in DCM to afford the title product (2) as a yellowish solid (35 mg, 47%).

### Example 3 Synthesis of (S)-1-(5-(5-((6-fluoropyridin-3-yl)carbamoyl)thiazol-2-yl)pyridin-2-yl)pyrrolidin-3-yl methanesulfonate Compound 3

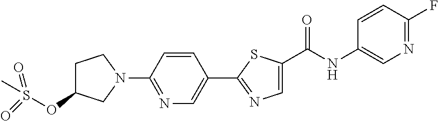 [SMILES: CS(=O)(=O)O[C@H]1CCN(c2ccc(-c3ncc(C(=O)Nc4ccc(F)nc4)s3)cn2)C1]

### Step-1: methyl 2-(6-fluoropyridin-3-yl) thiazole-5-carboxylate (B)

In an oven-dried round bottom flask was added (A) (3.0 g, 13 mmol), boronic acid (2.8 g, 20 mmol), NaHCO₃ (5.6 g, 65 mmol), and (THF:H₂O) (1:1, 45 mL, 15 vol) under an argon atmosphere. The reaction mixture was degassed with argon for 15 min. Then Pd(dppf)Cl₂·DCM (1.1 g, 13 mmol) was added and the mixture was heated to 90° C. for 16 h. The reactants were consumed as monitored by TLC. The reaction mixture was then quenched with ice-water (30 mL) and extracted in DCM (60 mL×3). The organic layer was dried over Na₂SO₄, concentrated and purified by silica gel chromatography (230-400 mesh) and eluted in 20% EtOAc in hexane to get (B) as an off white solid (1.6 g, 50%). MS (ESI): 239.08 [M]⁺ 1H NMR (CDCl₃) δ 8.71 (s, 1H), 8.61 (m, 1H), 8.46 (s, 1H), 7.07 (dd, 1H), 3.95 (s, 3H).

### Step-2: methyl (S)-2-(6-(3-hydroxypyrrolidin-1-yl) pyridin-3-yl) thiazole-5-carboxylate (1)

Compound (B) (1.0 g, 4.2 mmol), (S)-pyrrolidin-3-ol (440 mg, 5.0 mmol), DIPEA (2.3 mL, 12.6 mmol), and n-butanol (10 mL, 10 vol) was taken in an oven-dried microwave vial under argon atmosphere. The reaction mixture was irradiated at 120° C. for 2 h. After completion, solvent was removed under high vacuum. The obtained crude mass was purified by column chromatography over silica gel (230-400 mesh) and eluted in 2% MeOH in DCM to afford (1) as a white solid (100 mg, 69%). MS (ESI): 306.13 [M+H]⁺; 1H NMR (DMSO-D₆) δ 8.75 (d, 1H), 8.39 (d, 1H), 8.04 (dd, 1H), 6.57 (d, 1H), 5.03 (d, 1H), 4.41 (s, 1H), 3.85 (s, 2H), 3.54 (m, 3H), 2.03 (m, 1H), 1.93 (m, 1H), 1.23 (d, 1H).

### Step-3: (S)-2-(6-(3-hydroxypyrrolidin-1-yl) pyridin-3-yl) thiazole-5-carboxylic acid (J)

To a solution of (1) (1.3 g, 4.2 mmol) in (THF:MeOH:water) (2:2:1, 45 mL, 35 vol) was added lithium hydroxide (2.4 mg, 8.5 mmol) and the mixture was kept at RT for 6 h. Then the mixture was treated with 2M HCl (aq) solution until the pH reached up to 2-3. The biphasic mixture was stirred for 5 min and the layers were separated. The aqueous layer was further extracted with 10% MeOH in DCM (3×50 mL) and the combined organic layers were dried over Na₂SO₄ and concentrated under vacuum to get (J) as a brown solid (1.0 g, 83%). MS (ESI): 292.14 [M+H]⁺; 1H NMR (DMSO-D6) δ 13.54 (s, 1H), 8.74 (d, 1H), 8.30 (s, 1H), 8.03 (dd, 1H), 6.57 (d, 1H), 5.03 (s, 1H), 4.41 (s, 1H), 3.50 (m, 4H), 2.04 (m, 1H), 1.92 (m, 1H).

### Step-4: (S)—N-(6-fluoropyridin-3-yl)-2-(6-(3-hydroxypyrrolidin-1-yl) pyridin-3-yl) thiazole-5-carboxamide (K)

To a solution of (J) (80 mg, 0.27 mmol) in DMF (1.6 mL, 20 vol) was added HATU (208 mg, 0.54 mmol), followed by DIPEA (0.14 mL, 0.82 mmol) at 0° C. The resulting mixture was stirred at RT for 20 min. The reaction mixture was again cooled to 0° C. and to this was added 6-fluoropyridin-3-amine (36 mg, 0.33 mmol). The resulting mixture was stirred at RT for 24 h. After completion, the reaction mixture was quenched with ice-cool water and the product was extracted with 10% MeOH in DCM (20 mL×3). The extract was dried over Na₂SO₄ and concentrated under vacuum. The residue was purified by column chromatography over silica gel (230-400 mesh) and eluted in 5% MeOH in DCM to afford the product (K) as a yellowish solid (65 mg, 61%). LCMS (ESI): 386 [M]*; 1H NMR (DMSO-D6) δ 10.66 (s, 1H), 8.74 (d, 1H), 8.56 (s, 1H), 8.53 (s, 1H), 8.27 (m, 1H), 8.05 (dd, 1H), 7.23 (dd, 1H), 6.58 (d, 1H), 5.02 (d, 1H), 4.42 (s, 1H), 3.55 (dd, 3H), 3.41 (m, 1H), 2.04 (s, 1H), 1.94 (s, 1H)

### Step 5: (S)-1-(5-(5-((6-fluoropyridin-3-yl) carbamoyl) thiazol-2-yl) pyridin-2-yl) pyrrolidin-3-yl methane sulfonate (3)

To an ice-cold solution of (K) (65 mg, 0.16 mmol) in pyridine (2.0 mL, 33 vol.) was added methane sulfonyl chloride (0.3 mL, 4.5 mmol) portion wise at 0° C. under N₂ atmosphere and the mixture was stirred at RT for 2 h. The reaction time was monitored by TLC. After completion, the reaction mixture was cooled to 0° C. and saturated NaHCO₃ (2 mL) was added. The solvent was extracted with DCM (15 mL×3). The combined organic layers were washed with cold brine solution (7 mL×2), dried over Na₂SO₄ and concentrated under vacuum. The obtained crude mass was purified by column chromatography over silica gel (230-400 mesh) and eluted in 4% MeOH in DCM to afford (3) as a yellow solid (35 mg, 44%). MS (ESI): 392.26 (M+H)+. 1H NMR (DMSO-D₆) δ 10.68 (s, 1H), 8.77 (d, 1H), 8.58 (s, 1H), 8.53 (s, 1H), 8.27 (m, 1H), 8.11 (dd, 1H), 7.24 (dd, 1H), 6.6 (d, 1H), 5.45 (m, 1H), 3.7 (m, 3H), 3.5 (m, 1H), 3.2 (s, 3H), 2.39 (m, 2H),

### Example 4 Synthesis of tritiated 2-[2-fluoro-6-[(3R)-3-fluoropyrrolidin-1-yl]-3-pyridyl]-5-(3-pyridyl)-6,7-dihydrothiazolo[5,4-c]pyridin-4-one ³H-Compound 1

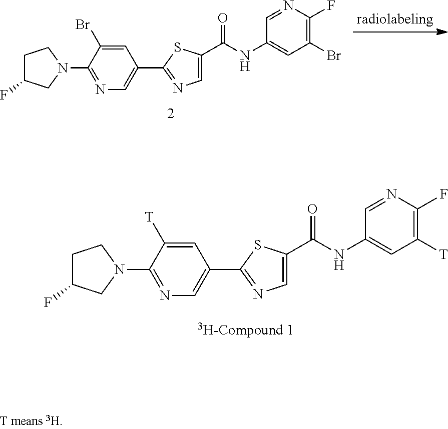

2.88 mg of the dibromo precursor (2), 2.97 mg of Pd/C(10% metal) and 50 μl DIPEA were suspended in 0.3 ml DMF. The suspension was degassed three times at the high vacuum manifold and stirred under an atmosphere of tritium gas (5.3 Ci) for 2.5 h at room temperature. The solvent was removed in vacuo, and labile tritium was exchanged by adding 0.3 ml of methanol, stirring the solution, and removing the solvent again under vacuo. This process was repeated twice. The dried solid was extracted with 10 ml methanol/DCM (1:1) and the suspension was filtered through a 0.2 μm nylon membrane, obtaining a clear solution. For the purification of the compound, the following HPLC conditions were used: Waters Sunfire C18, 10×250 mm; solvents A: water(+0.1% TFA (Sigma-Aldrich T6508); B: acetonitrile+0.1% TFA. The purified product ³H-Compound 1 was obtained (SA 49.1 Ci/mmol, 99% purity).

### Example 5 Synthesis of fluorinated 2-[2-fluoro-6-[(3R)-3-fluoropyrrolidin-1-yl]-3-pyridyl]-5-(3-pyridyl)-6,7-dihydrothiazolo[5,4-c]pyridin-4-one ¹⁸F-Compound 1

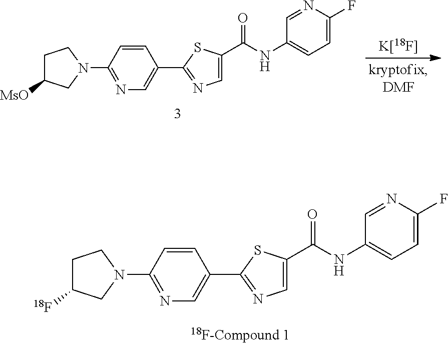

A solution of (S)-1-(5-(5-((6-fluoropyridin-3-yl)carbamoyl)thiazol-2-yl)pyridin-2-yl)pyrrolidin-3-yl methane sulfonate (3) (1 mg in 1 mL anhydrous DMSO) was added to a microwave vial containing dry [¹⁸F]fluoride and the vessel was heated at 110° C. for 20 min. The reaction vessel was cooled to 40° C. and the mixture was diluted with HPLC buffer (4 mL). The resulting mixture was passed through a Sep-Pak® Alumina-N Light Cartridge. Crude material was purified by semi-preparative HPLC and the collected purified fraction was passed through a Strata® C18-E cartridge. The final product ¹⁸F-Compound 1 was filtered through a sterilizing filter onto the final product vial inside the dispensing hot cell. The final product vial was assayed, and sample was removed for QC testing. Confirmation of the identity of the product was determined by co-injection with a sample of the ¹⁹F-reference compound

### Example Compounds 4 to 11

Following the coupling procedure as reported for the preparation of Example 1 and utilizing the building blocks and halogen derivatives indicated in Table 1, the following compounds were prepared.

TABLE 1

| | | | | 1. Yield |
|---|---|---|---|---|
| | | | | 2. ¹H NMR |
| | | | | 3. MH⁺ (ESI) |
| | | Aryl | | 4. Synthesis |
| Example | Building block | amines | Product | procedure |
| 4 | 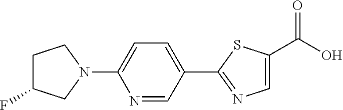 [SMILES: O=C(O)c1cnc(-c2ccc(N3CC[C@@H](F)C3)nc2)s1] | 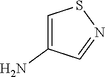 | 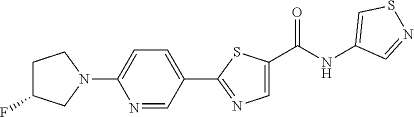 [SMILES: O=C(Nc1cnsc1)c1cnc(-c2ccc(N3CC[C@@H](F)C3)nc2)s1] | 1. Yield % = 17% 2. 1H NMR (500 MHz, DMSO-d6): δ 11.13 (s, 1H), 8.98 (s, 1H), 8.72 (m, 2H), 8.51 (s, 1H), 8.06 (dd, 1H), 6.61 (d, 1H), 5.44 (d, 1H), |
| | | | | 3.71 (m, 3H), 3.47 |
| | | | | (dd, 1H), 2.20 (m, |
| | | | | 2H). |
| | | | | 3. LCMS (ESI) = |
| | | | | 377.8 (M + H)+ |
| 5 | 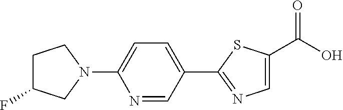 [SMILES: O=C(O)c1cnc(-c2ccc(N3CC[C@@H](F)C3)nc2)s1] | 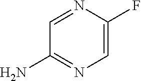 | 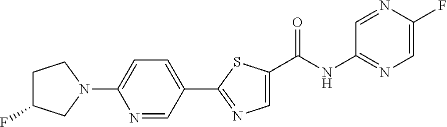 [SMILES: O=C(Nc1cnc(F)cn1)c1cnc(-c2ccc(N3CC[C@@H](F)C3)nc2)s1] | 1. Yield % = 15% 2. ¹H NMR (500 MHz, DMSO-d₆): δ 11.59 (s, 1H), 9.00 (s, 1H), 8.81 (s, 1H), 8.78 (d, 1H), 8.57 (d, 1H), 8.10 (dd, 1H), 6.65 (d 1H), 5.48 (d, 1H), |
| | | | | 3.69 (m, 3H), 3.51 |
| | | | | (dd, 1H), 2.24 (m, |
| | | | | 2H). |
| | | | | 3. LCMS (ESI) = 389 |
| | | | | (M + H)₊ |
| 6 | 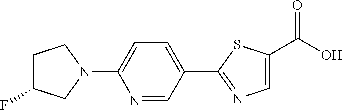 [SMILES: O=C(O)c1cnc(-c2ccc(N3CC[C@@H](F)C3)nc2)s1] | 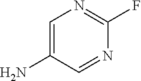 | 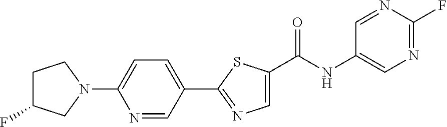 [SMILES: O=C(Nc1cnc(F)nc1)c1cnc(-c2ccc(N3CC[C@@H](F)C3)nc2)s1] | 1. Yield % = 22% 2. ¹H NMR (500 MHz, DMSO-d₆): δ 10.92 (s, 1H), 9.05 (s, 2H), 8.78 (d, 1H), 8.59 (s, 1H), 8.10 (dd, 1H), 6.65 (d, 1H), 5.48 (d, 1H), 3.75 (m, 3H), |
| | | | | 3.51 (td, 1H), 2.28 |
| | | | | (m, 2H). |
| | | | | 3. LCMS (ESI) = 389 |
| | | | | (M + H)₊ |
| 7 | 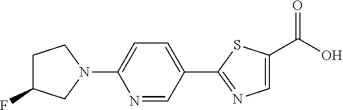 [SMILES: O=C(O)c1cnc(-c2ccc(N3CC[C@H](F)C3)nc2)s1] | 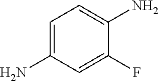 | 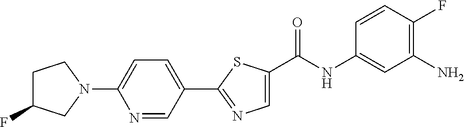 [SMILES: Nc1cc(NC(=O)c2cnc(-c3ccc(N4CC[C@H](F)C4)nc3)s2)ccc1F] | 1. Yield % = 80% 2. ¹H NMR (400 MHz, DMSO-d₆): δ 10.30 (s, 1H), 8.66 (d, 1H), 8.58 (s, 1H), 8.15 (dd, 1H), 7.34 (dd, 1H), 7.01 (dd, 1H), 6.94 (d, 1H), 6.76 (d, |
| | | | | 1H), 5.46 (d, 1H), |
| | | | | 3.95 (s, 2H), 3.65 (m, |
| | | | | 4H), 2.19 (m, 2H). |
| | | | | 3. LCMS (ESI) = 402.2 |
| | | | | (M + H)₊ |
| 8 | 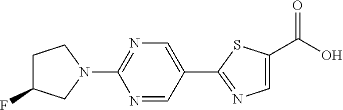 [SMILES: O=C(O)c1cnc(-c2cnc(N3CC[C@H](F)C3)nc2)s1] | 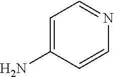 | 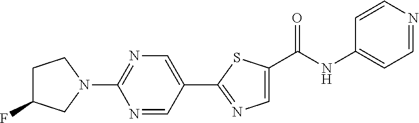 [SMILES: O=C(Nc1ccncc1)c1cnc(-c2cnc(N3CC[C@H](F)C3)nc2)s1] | 1. Yield % = 80% 2. ¹H NMR (500 MHz, DMSO-d₆): δ 10.74 (s, 1H), 8.99 (d, 2H), 8.69 (s, 1H), 8.51 (s, 2H), 7.73 (s, 2H), 5.48 (d, 1H), 3.81 (m, 3H), 3.59 (m, 1H), |
| | | | | 2.24 (m, 2H). |
| | | | | 3. LCMS (ESI) = |
| | | | | 370.9 (M + H)₊ |
| 9 | 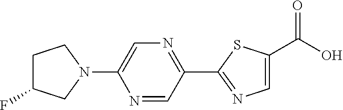 [SMILES: O=C(O)c1cnc(-c2cnc(N3CC[C@@H](F)C3)cn2)s1] | 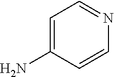 | 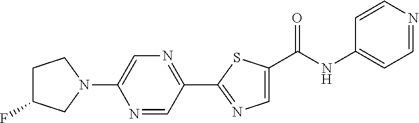 [SMILES: O=C(Nc1ccncc1)c1cnc(-c2cnc(N3CC[C@@H](F)C3)cn2)s1] | 1. Yield % = 80% 2. ¹H NMR (500 MHz, DMSO-d₆): δ 10.71 (s, 1H), 8.85 (s, 1H), 8.67 (s, 1H), 8.50 (d, 2H), 8.14 (s, 1H), 7.73 (d, 2H), 5.52 (d, 1H), 3.82 (m, 3H), |
| | | | | 3.58 (dd, 1H), 2.26 |
| | | | | (m, 2H). |
| | | | | 3. LCMS (ESI) = |
| | | | | 370.9 (M + H)₊ |
| 10 | 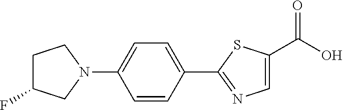 [SMILES: O=C(O)c1cnc(-c2ccc(N3CC[C@@H](F)C3)cc2)s1] | 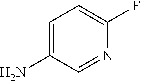 | 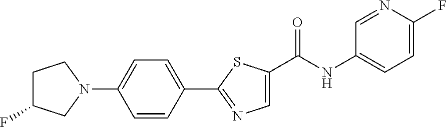 [SMILES: O=C(Nc1ccc(F)nc1)c1cnc(-c2ccc(N3CC[C@@H](F)C3)cc2)s1] | 1. Yield % = 19% 2. ¹H NMR (400 MHz, DMSO-d₆): δ 10.63 (s, 1H), 8.53 (m, 2H), 8.27 (m, 1H), 7.86 (d, 2H), 7.23 (dd, 1H), 6.69 (d, 2H), 5.49 (d, 1H), 3.60 (m, 3H), |
| | | | | 3.44 (m, 1H), 2.21 |
| | | | | (m, 2H). |
| | | | | 3. LCMS (ESI) = |
| | | | | 387.30 (M + H)₊ |
| 11 | 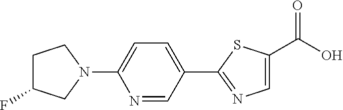 [SMILES: O=C(O)c1cnc(-c2ccc(N3CC[C@@H](F)C3)nc2)s1] | 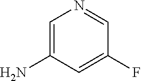 | 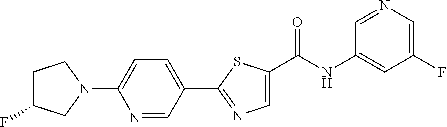 [SMILES: O=C(Nc1cncc(F)c1)c1cnc(-c2ccc(N3CC[C@@H](F)C3)nc2)s1] | 1. Yield % = 87% 2. ¹H NMR (400 MHz, DMSO-d₆): δ 11.13 (s, 1H), 8.84 (t, 1H), 8.81 (d, 1H), 8.70 (d, 1H), 8.38 (d, 1H), 8.24 (m, 2H), 6.90 (d, 1H), 5.52 (m, 1H), |
| | | | | 3.83 (m, 3H), 3.59 |
| | | | | (m, 1H), 2.29 (m, |
| | | | | 2H). |
| | | | | 3. LCMS |
| | | | | (ESI) = 387.8 (M + H)₊ |

### Synthesis of Example Compounds 16 to 18 (Precursors of ³H Compounds 4, 8 and 11 Respectively)

Following the coupling procedure as reported for the preparation of Example 2 and utilizing the building blocks and halogen derivatives indicated in Table 2, the following compounds were prepared.

TABLE 2

| | | | | 1. Yield |
|---|---|---|---|---|
| | | | | 2. ¹H NMR |
| | | | | 3. MH⁺ (ESI) |
| | | Aryl | | 4. Synthesis |
| Example | Building block | amines | Product | procedure |
| 16 | 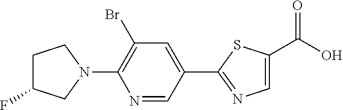 [SMILES: O=C(O)c1cnc(-c2cnc(N3CC[C@@H](F)C3)c(Br)c2)s1] | 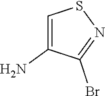 | 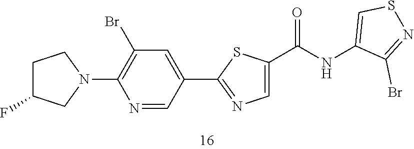 [SMILES: O=C(Nc1csnc1Br)c1cnc(-c2cnc(N3CC[C@@H](F)C3)c(Br)c2)s1] | 1. Yield % = 45% 2. 1H NMR (500 MHz, DMSO-d6): δ 10.44 (s, 1H), 9.09 (s, 1H), 8.75 (d, 1H), 8.63 (s, 1H), 8.37 (d, 1H), 5.44 (d, 1H), 4.06 (dd, 1H), 3.94-3.83 (m, |
| | | | Precursor of ³H-compound 4 | 3H), 2.29-2.05 (m, |
| | | | | 2H) |
| | | | | 3. LCMS (ESI) = |
| | | | | 534.1 (M + H)+ |
| 17 | 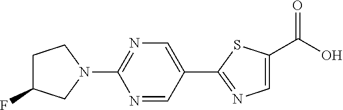 [SMILES: O=C(O)c1cnc(-c2cnc(N3CC[C@H](F)C3)nc2)s1] | 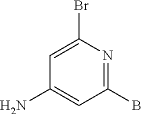 | 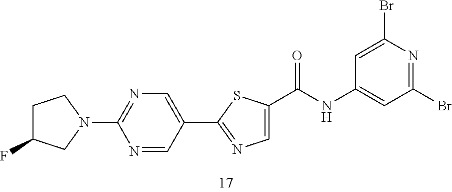 [SMILES: O=C(Nc1cc(Br)nc(Br)c1)c1cnc(-c2cnc(N3CC[C@H](F)C3)nc2)s1] | 1. Yield % = 10% 2. 1H NMR (500 MHz, DMSO-d6): δ 10.98 (s, 1H), 8.99 (d, 2H), 8.65 (s, 1H), 8.00 (s, 2H), 5.54-5.40 (m, 1H), 3.92-3.83 (m, 3H), 3.60-3.58 (m, 1H), 2.31-2.15 (m, 2H). 3. LCMS (ESI) = 528.9 (M + H)+ |
| | | | Precursor of ³H-compound 8 | |
| 18 | 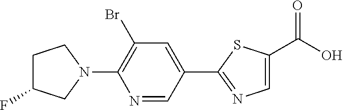 [SMILES: O=C(O)c1cnc(-c2cnc(N3CC[C@@H](F)C3)c(Br)c2)s1] | 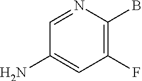 | 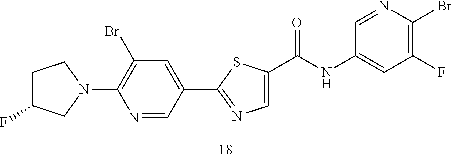 [SMILES: O=C(Nc1cnc(Br)c(F)c1)c1cnc(-c2cnc(N3CC[C@@H](F)C3)c(Br)c2)s1] | 1. Yield % = 18% 2. 1H NMR (500 MHz, DMSO-d6): δ 10.96 (s, 1H), 8.76 (d, 1H), 8.64 (s, 1H), 8.59 (d, 1H), 8.37 (d, 1H), 8.26 (dd, 1H), 5.44 (d, 1H), 4.06 (dd, 1H), 3.90 (m, 3H), 2.19 |
| | | | Precursor of ³H-compound 11 | (m, 2H). |
| | | | | 3. LCMS (ESI) = |
| | | | | 546.5 (M + H)+ |

### Example 6 Synthesis of (R)-4-fluoro-N-(5-fluoropyridin-3-yl)-2-(6-(3-fluoropyrrolidin-1-yl)pyridin-3-yl)thiazole-5-carboxamide Compound 12

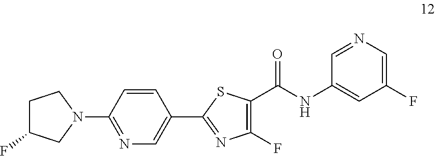 [SMILES: O=C(Nc1cncc(F)c1)c1sc(-c2ccc(N3CC[C@@H](F)C3)nc2)nc1F]

### Step-1: Synthesis of 2-bromo-4-fluorothiazole-5-carbonyl chloride (R)

To a solution of 2-bromo-4-fluorothiazole-5-carboxylic acid Q (0.3 g, 1.33 mmol) in THF (6 mL) was added (COCl)₂ (0.34 g, 2.65 mmol) and DMF (102.12 uL, 1.33 mmol). The mixture was stirred at 20° C. for 0.5 hr. The reaction mixture was concentrated under reduced pressure to afford title compound R (0.3 g, 92% yield) as a yellow solid.

### Step-2: Synthesis of 2-bromo-4-fluoro-N-(5-fluoropyridin-3-yl)thiazole-5-carboxamide (T)

To a solution of 5-fluoropyridin-3-amine (165.08 mg, 1.47 mmol) and compound R (300 mg, 1.23 mmol) in THF (8 mL) was added pyridine (297.14 uL, 3.68 mmol). The mixture was stirred for 2 hours at 20° C. The residue was poured into ice-water (100 mL), the aqueous phase was extracted with ethyl acetate (30 mL*3). The combined organic phase was washed with brine (30 mL*3), dried with anhydrous Na₂SO₄, filtered, and concentrated in vacuum to give a residue. The residue was purified by prep-TLC (PE:EA=1:1, Rf=0.5) to afford compound T (300 mg, 69% yield) as a white solid. 1H NMR (400 MHz, DMSO-d6) δ=10.65 (s, 1H), 8.68 (s, 1H), 8.41-8.37 (m, 1H), 8.07-8.00 (m, 1H)

### Step-3: Synthesis of (R)-4-fluoro-N-(5-fluoropyridin-3-yl)-2-(6-(3-fluoropyrrolidin-1-yl)pyridin-3-yl)thiazole-5-carboxamide (Compound 12)

A mixture of compound 3 (200 mg, 624.78 μmol), (R)-(6-(3-fluoropyrrolidin-1-yl)pyridin-3-yl)boronic acid (182.53 mg, 624.78 umol), XPhos Pd G₂ (491.58 mg, 624.78 umol), K₃PO₄ (397.86 mg, 1.87 mmol) in H₂O (0.2 mL) and Tol. (1 mL) was degassed and purged with N₂ for 3 times, and then the mixture was stirred at 70° C. for 0.5 hr under N₂ atmosphere. The residue was poured into sat. EDTA (100 mL) and stirred for 2 h. The aqueous phase was extracted with ethyl acetate (30 mL*3). The combined organic phase was washed with brine (30 mL*3), dried with anhydrous Na₂SO₄, filtered, and concentrated in vacuum. The crude was purified by prep-HPLC column: Phenomenex C18 75*30 mm*3 um; mobile phase: [water(HCl)-ACN]; B %: 15%-55%, 8 min) to afford compound 12 (50 mg, 19% yield) as a yellow solid.

1H NMR (400 MHz, DMSO-d6) δ=10.65 (s, 1H), 8.77 (s, 1H), 8.68-8.61 (m, 1H), 8.44-8.37 (m, 1H), 8.23-8.10 (m, 2H), 6.86 (d, J=9.2 Hz, 1H), 5.63-5.41 (m, 1H), 4.11-3.68 (m, 3H), 3.65-3.52 (m, 1H), 2.43-2.12 (m, 2H) LCMS: 406.1 [M+H]⁺

### Example 7 Synthesis of (R)-4-fluoro-N-(6-fluoropyridin-3-yl)-2-(6-(3-fluoropyrrolidin-1-yl)pyridin-3-ylthiazole-5-carboxamide Compound 13

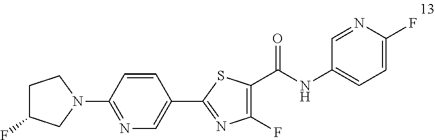 [SMILES: O=C(Nc1ccc(F)nc1)c1sc(-c2ccc(N3CC[C@@H](F)C3)nc2)nc1F]

### Step-1: Synthesis of tert-butyl (4-chloro-5-formylthiazol-2-yl)carbamate (M)

To a solution of 2-amino-4-chlorothiazole-5-carbaldehyde L (10 g, 61.50 mmol) in dioxane (100 mL) was added DMAP (751.35 mg, 6.15 mmol) and (Boc)₂O (15.54 mL, 67.65 mmol). The mixture was heated to 60° C. and stirred for 3 hours. The mixture was cooled to 25° C., poured into ice-water (200 mL). The aqueous phase was extracted with ethyl acetate (50 mL*3). The combined organic phase was washed with brine (50 mL*3), dried over anhydrous Na₂SO₄, filtered and concentrated in vacuum to give a residue. The residue was purified by silica gel chromatography (Petroleum ether/Ethyl acetate=10/1 to 5/1) to afford title compound M (12 g, 74% yield) as a yellow solid.

¹H NMR (400 MHz, DMSO-d6) δ=9.85 (s, 1H), 1.50 (s, 9H)

LCMS: 263.0 [M+H]⁺

### Step-2: Synthesis of tert-butyl N-(4-fluoro-5-formylthiazol-2-yl) carbamate (N)

To the solution of compound M (10 g, 38.06 mmol) in DMSO (100 mL) was added CsF (115.64 g, 761.29 mmol). The mixture was heated to 130° C. and stirred for 48 hours. The reaction mixture was cooled to 25° C., poured into ice-water (1000 mL). The aqueous phase was extracted with ethyl acetate (300 mL*3). The combined organic phase was washed with brine (300 mL*3), dried over anhydrous Na₂SO₄, filtered and concentrated in vacuum to give a residue. The residue was purified by prep-HPLC (column: Phenomenex C18 75*30 mm*3 um; mobile phase: [water (NH₄HCO₃)-ACN]; B %: 10%-40%, 8 min) to afford title compound N (2.5 g, 27% yield) as a yellow solid.

¹H NMR (400 MHz, CDCl₃) δ=9.96 (s, 1H), 9.23-9.17 (m, 1H), 1.60 (s, 9H)

LCMS: 247.2 [M+H]⁺

### Step-3: Synthesis of 2-amino-4-fluorothiazole-5-carbaldehyde (O)

To a solution of compound N (2.5 g, 10.15 mmol) in DCM (50 mL) was added ZnBr₂ (6.86 g, 30.46 mmol). The mixture was heated to 40° C. and stirred for 16 hours. The mixture was cooled to 25° C., the reaction mixture was concentrated in vacuum to give a residue. The residue was purified by silica gel column chromatography (PE/EA=10:1/1:1) to afford title compound 0 (1.1 g, 74% yield) as a yellow solid.

1H NMR (400 MHz, DMSO-d6) δ=9.59 (s, 1H), 8.95 (s, 2H)

LCMS: 147.2 [M+H]⁺

### Step-4: Synthesis of 2-bromo-4-fluorothiazole-5-carbaldehyde (P)

To a solution of compound O (1 g, 6.84 mmol) in ACN (40 mL) was added CuBr₂ (1.53 g, 6.84 mmol) and a solution of isopenty nitrite (1.45 g, 14.03 mmol) in ACN (20 mL). The mixture was stirred at 65° C. for 0.5 hr. The reaction mixture was quenched by addition ice-water (100 mL) at 25° C. The aqueous phase was extracted with ethyl acetate (100 mL*3). The combined organic layers were washed with brine (50 mL*2), dried over anhydrous Na₂SO₄, filtered and concentrated under reduced pressure to give a residue. The residue was purified by column chromatography (Petroleum ether/Ethyl acetate=30/1 to 1/1) to afford title compound P (540 mg, 38% yield) as a white solid.

1H NMR (400 MHz, DMSO-d6) δ=9.87 (s, 1H)

### Step-5: Synthesis of 2-bromo-4-fluorothiazole-5-carboxylic acid (Q)

To a solution of compound P (500 mg, 2.38 mmol) in t-BuOH (4.2 mL) and H₂O (2.4 mL) was added 2-methylbut-2-ene (659.50 mg, 9.40 mmol) and NaH₂PO₄ (791.19 mg, 6.59 mmol) and NaClO₂ (987 mg, 10.91 mmol). The mixture was stirred at 20° C. for 0.5 hr. The reaction was poured into ice-water (50 mL). The aqueous phase was extracted with ethyl acetate (50 mL*3). Then the aqueous phase was neutralized with citric acid(sat.) to pH=5-6 and extracted with ethyl acetate (50 mL*3). The combined organic phase was washed with brine (100 mL*3), dried with anhydrous Na₂SO₄, filtered and concentrated in vacuum to afford title compound Q (400 mg, 74% yield) as a white solid.

1H NMR (400 MHz, DMSO-d6) δ=14.05 (brs, 1H)

### Step-6: Synthesis of 2-bromo-4-fluorothiazole-5-carbonyl chloride (R)

To a solution of compound Q (0.3 g, 1.33 mmol) in THF (6 mL) was added (COCl)₂ (0.34 g, 2.65 mmol) and DMF (102.12 uL, 1.33 mmol). The mixture was stirred at 20° C. for 0.5 hr. The reaction mixture was concentrated under reduced pressure to afford title compound R (0.3 g, 92% yield) as a yellow solid.

### Step-7: Synthesis of 2-bromo-4-fluoro-N-(6-fluoropyridin-3-yl)thiazole-5-carboxamide (S)

To a solution of 6-fluoropyridin-3-amine (150 mg, 1.34 mmol) and compound R (270 mg, 1.10 mmol) in THF (2 mL) was added pyridine (647.99 uL, 8.03 mmol). The mixture was stirred for 0.5 hours at 20° C. The residue was poured into ice-water (100 mL), the aqueous phase was extracted with ethyl acetate (30 mL*3). The combined organic phase was washed with brine (30 mL*3), dried with anhydrous Na₂SO₄, filtered and concentrated in vacuum to give a residue. The residue was purified by prep-TLC (SiO₂, PE:EA=1:1, Rf=0.5) to afford compound S (300 mg, 70% yield) as a white solid.

1H NMR (400 MHz, DMSO-d6) δ=10.49 (s, 1H), 8.46 (s, 1H), 8.23-8.14 (m, 1H), 7.26-7.18 (m, 1H)

### Step-8: Synthesis of (R)-4-fluoro-N-(6-fluoropyridin-3-yl)-2-(6-(3-fluoropyrrolidin-1-yl)pyridin-3-yl)thiazole-5-carboxamide (Compound 13)

A mixture of compound S (160 mg, 499.82 μmol), (R)-(6-(3-fluoropyrrolidin-1-yl)pyridin-3-yl)boronic acid (219.04 mg, 749.73 umol), Pd(dppf)Cl₂ (18.29 mg, 24.99 umol), K₃PO₄ (318.29 mg, 1.50 mmol) in H₂O (1 mL) and Tol. (5 mL) was degassed and purged with N₂ for 3 times, and then the mixture was stirred at 70° C. for 0.5 hrs under N₂ atmosphere. The residue was poured into saturated sat. EDTA (100 mL) and stirred for 1 h. The aqueous phase was extracted with ethyl acetate (30 mL*3). The combined organic phase was washed with brine (30 mL*3), dried with anhydrous Na₂SO₄, filtered and concentrated in vacuum. The crude was purified by prep-HPLC (column: Phenomenex luna C18 250*50 mm*10 um; mobile phase: [water(HCl)-ACN]; B %: 20%-50%, 10 min) to afford compound 13 (50 mg, 24.31% yield) as a yellow solid.

1H NMR (400 MHz, DMSO-d6) δ=10.33 (s, 1H), 8.71 (s, 1H), 8.49 (s, 1H), 8.27-8.19 (m, 1H), 8.13-8.06 (m, 1H), 7.23-7.15 (m, 1H), 6.73 (d, J=9.2 Hz, 1H), 5.60-5.40 (m, 1H), 3.92-3.61 (m, 3H), 3.60-3.48 (m, 1H), 2.39-2.10 (m, 2H)

LCMS: 406.1 [M+H]⁺

### Example Compounds 14 to 15

Following the coupling procedure as reported for the preparation of Example 13 and utilizing the building blocks and halogen derivatives indicated in Table 3, the following compounds were prepared.

TABLE 3

| | | | | 1. Yield |
|---|---|---|---|---|
| | | | | 2. ¹H NMR |
| | | | | 3. MH⁺ (ESI) |
| Ex- | | Halogen | | 4. Synthesis |
| ample | Building block | derivatives | Product | procedure |
| 14 | 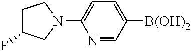 [SMILES: OB(O)c1ccc(N2CC[C@@H](F)C2)nc1] | 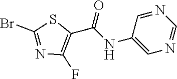 [SMILES: O=C(Nc1cncnc1)c1sc(Br)nc1F] | 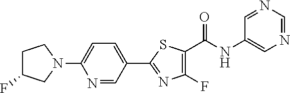 [SMILES: O=C(Nc1cncnc1)c1sc(-c2ccc(N3CC[C@@H](F)C3)nc2)nc1F] | 1. Yield % = 25.6% 2. 1H NMR (500 MHz, DMSO-d6): δ = 10.52 (s, 1 H), 9.10 (s, 2 H), 8.96 (s, |
| | | | | 1 H), 8.73-8.62 |
| | | | | (m, 1 H), 8.24- |
| | | | | 8.12 (m, 1 H), |
| | | | | 6.85 (d, J = 9.2 Hz, |
| | | | | 1 H), 5.64-5.41 (m, |
| | | | | 1 H), 4.11-3.67 (m, |
| | | | | 3 H), 3.65-3.52 (m, |
| | | | | 1 H), 2.44-2.13 (m, |
| | | | | 2 H) |
| | | | | 3. LCMS (ESI) = |
| | | | | 389.1 (M + H)+ |
| 15 | 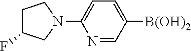 [SMILES: OB(O)c1ccc(N2CC[C@@H](F)C2)nc1] | 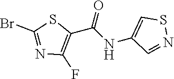 [SMILES: O=C(Nc1cnsc1)c1sc(Br)nc1F] | 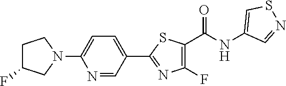 [SMILES: O=C(Nc1cnsc1)c1sc(-c2ccc(N3CC[C@@H](F)C3)nc2)nc1F] | 1. Yield % = 8.5% 2. ¹H NMR (500 MHz, DMSO-d₆): δ = 10.73 (s, 1H), 9.02 (s, 1H), 8.77 (s, |
| | | | | 1H), 8.72 (s, 1H), |
| | | | | 8.07 (d, J = 8.8 Hz, |
| | | | | 1H), 6.70 (d, J = |
| | | | | 8.8 Hz, 1H), 5.59- |
| | | | | 5.39 (m, 1H), 3.93- |
| | | | | 3.73 (m, 3H), 3.53- |
| | | | | 3.47 (m, 1H), 2.36- |
| | | | | 2.13 (m, 2H) |
| | | | | 3. LCMS (ESI) = |
| | | | | 394 (M + H)₊ |

### Example 8 Synthesis of (R)—N-(2,4-dibromo-5-fluoropyridin-3-yl)-4-fluoro-2-(6-(3-fluoropyrrolidin-1-yl)pyridin-3-yl)thiazole-5-carboxamide Compound 19 (Precursor of ³H-Compound 12)

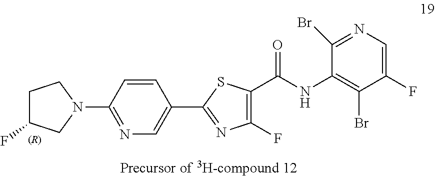

### Step-1: Synthesis of (R)-4-fluoro-2-(6-(3-fluoropyrrolidin-1-yl)pyridin-3-yl)thiazole-5-carboxylic acid (V)

To a mixture of (R)-2-(3-fluoropyrrolidin-1-yl)-5-(4,4,5,5-tetramethyl-1,3,2-dioxaborolan-2-yl)pyridine (775.60 mg, 2.65 mmol) and 2-bromo-4-fluorothiazole-5-carboxylic acid U (500 mg, 2.21 mmol) in dioxane (10 mL), H₂O (1 mL) was added Pd(dppf)Cl₂ (161.86 mg, 221.22 umol), Cs₂CO₃ (1.44 g, 4.42 mmol) at 25° C. The reaction mixture was stirred at 110° C. for 2 h under N₂. LCMS showed 41% desired was detected. The reaction mixture was stirred with palladium scavenger (100 mg), filtered and concentrated in vacuum. The residue was purified by prep-HPLC (HCl condition: column: Phenomenex luna C18 80*40 mm*3 um; mobile phase: [water(HCl)-ACN]; gradient: 15%-45% B over 7 min), then lyophilization to afford title compound V (200 mg, 29% yield) as yellow solid.

¹H NMR (400 MHz, DMSO-d6) δ=13.56 (br s, 1H), 8.73 (s, 1H), 8.06-7.96 (m, 1H), 6.63 (d, J=8.8 Hz, 1H), 5.59-5.36 (m, 1H), 3.85-3.48 (m, 4H), 2.33-2.15 (m, 2H)

LCMS: 312.1 [M+H]⁺

### Step-2: Synthesis of (R)-4-fluoro-2-(6-(3-fluoropyrrolidin-1-yl)pyridin-3-yl)thiazole-5-carbonyl chloride (W)

To a mixture of compound V (200 mg, 642.46 μmol) in DCM was added SOCl₂ (2 mL). The mixture was stirred at 70° C. for 1 hour. LCMS showed the reaction was 89% desired MS (quenched by MeOH). The reaction mixture was concentrated in vacuum. To afford compound W (200 mg, crude) as yellow solid.

LCMS: 326.2 (quenched by MeOH)

### Step-3: Synthesis of (R)—N-(2,4-dibromo-5-fluoropyridin-3-yl)-4-fluoro-2-(6-(3-fluoropyrrolidin-1-yl)pyridin-3-yl)thiazole-5-carboxamide (Compound 19)

To a mixture of 2,4-dibromo-5-fluoropyridin-3-amine (163.70 mg, 606.52 umol) in THE (2 mL) was added NaH (72.77 mg, 1.82 mmol, 60% purity) at 0° C. The mixture was stirred at 0° C. for 0.5 hours. Then compound W (200 mg, 606.52 umol) was added, the mixture was stirred at 25° C. for 1.5 h. LCMS showed the reaction was 32% desired MS. The reaction mixture was poured into sat. aq. NH₄Cl (60 mL). The aqueous phase was extracted with ethyl acetate (20 mL*3). The combined organic phase was washed with brine (20 mL*3), dried with anhydrous Na₂SO₄, filtered, and concentrated in vacuum. The residue was purified by prep-TLC (SiO₂, PE:EA=2:1). To afford compound 20 (30.12 mg, 8.55% yield) as yellow solid. 1H NMR (400 MHz, DMSO-d6): 5=10.34 (br s, 1H), 8.79-8.73 (m, 1H), 8.57 (s, 1H), 8.09-8.01 (m, 1H), 6.66 (d, J=8.8 Hz, 1H), 5.58-5.38 (m, 1H), 3.92-3.45 (m, 4H), 2.38-2.11 (m, 2H)

LCMS: 563.9 [M+H]⁺

### Example 9 Synthesis of tritiated (R)-2-(6-(3-fluoropyrrolidin-1-yl)pyridin-3-yl)-N-(isothiazol-4-yl)thiazole-5-carboxamide ³H-Compound 4

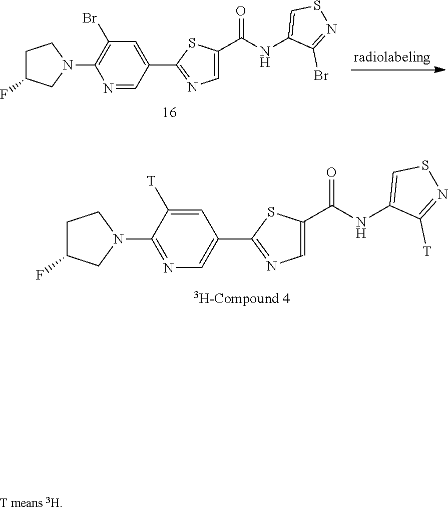

3.54 mg of the dibromo precursor (16), 9.9 mg of Pd/C(10% metal) and 50 μl DIPEA were suspended in 0.3 ml DMF. The suspension was degassed three times at the high vacuum manifold and stirred under an atmosphere of tritium gas (5.1 Ci) for 16 h at room temperature. The solvent was removed in vacuo, and labile tritium was exchanged by adding 0.3 ml of methanol, stirring the solution, and removing the solvent again under vacuo. This process was repeated twice. The dried solid was extracted with 10 ml methanol/DCM (1:1) and the suspension was filtered through a 0.2 μm nylon membrane, obtaining a clear solution. For the purification of the compound, the following HPLC conditions were used: Waters Sunfire C18, 10×250 mm; solvents A: water(+0.1% TFA (Sigma-Aldrich T6508); B: acetonitrile+0.1% TFA. The purified product ³H-Compound 4 was obtained (SA 43.5 Ci/mmol, 99% purity).

### Example 10 Synthesis of tritiated (S)-2-(2-(3-fluoropyrrolidin-1-yl)pyrimidin-5-yl)-N-(pyridin-4-yl)thiazole-5-carboxamide ³H-Compound 8

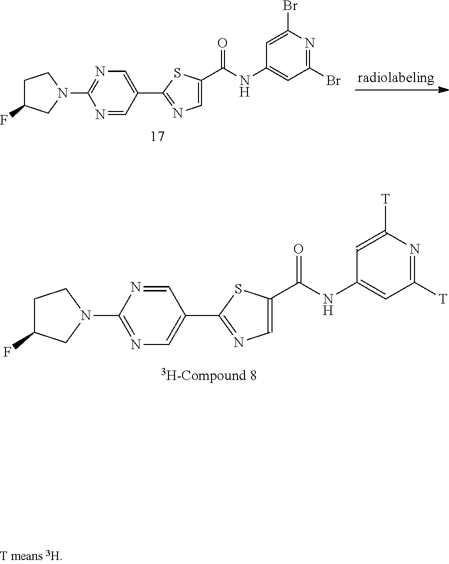

2.56 mg of the dibromo precursor (17), 11.2 mg of Pd/C(10% metal) and 20 μl DIPEA were suspended in 0.3 ml DMF. The suspension was degassed three times at the high vacuum manifold and stirred under an atmosphere of tritium gas (5.7 Ci) for 50 mn at room temperature. The solvent was removed in vacuo, and labile tritium was exchanged by adding 0.3 ml of methanol, stirring the solution, and removing the solvent again under vacuo. This process was repeated twice. The dried solid was extracted with 10 ml methanol/DCM (1:1) and the suspension was filtered through a 0.2 μm nylon membrane, obtaining a clear solution. For the purification of the compound, the following HPLC conditions were used: Waters Sunfire C18, 10×250 mm; solvents A: water(+0.1% TFA (Sigma-Aldrich T6508); B: acetonitrile+0.1% TFA. The purified product ³H-Compound 8 was obtained (SA 42.5 Ci/mmol, 99% purity).

### Example 11 Synthesis of tritiated (R)—N-(5-fluoropyridin-3-yl)-2-(6-(3-fluoropyrrolidin-1-yl)pyridin-3-yl)thiazole-5-carboxamide ³H-Compound 11

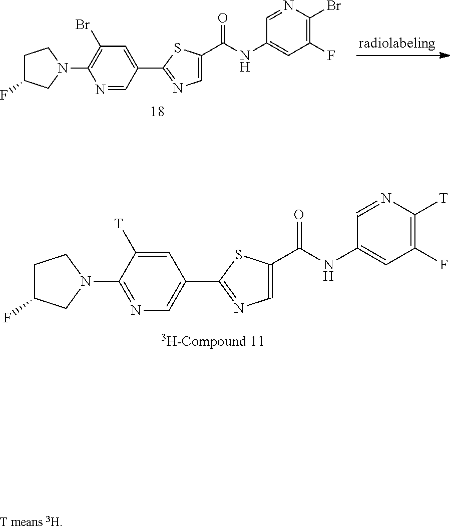

2.95 mg of the dibromo precursor (18), 17 mg of Pd/C(10% metal) and 20 μl DIPEA were suspended in 0.3 ml DMF. The suspension was degassed three times at the high vacuum manifold and stirred under an atmosphere of tritium gas (9.1 Ci) for 15 mn at room temperature. The solvent was removed in vacuo, and labile tritium was exchanged by adding 0.3 ml of methanol, stirring the solution, and removing the solvent again under vacuo. This process was repeated twice. The dried solid was extracted with 10 ml methanol/DCM (1:1) and the suspension was filtered through a 0.2 μm nylon membrane, obtaining a clear solution. For the purification of the compound, the following HPLC conditions were used: Waters Sunfire C18, 10×250 mm; solvents A: water(+0.1% TFA (Sigma-Aldrich T6508); B: acetonitrile+0.1% TFA. The purified product ³H-Compound 11 was obtained (SA 44.3 Ci/mmol, 99% purity).

### Example 12 Synthesis of tritiated (R)—N-(5-fluoropyridin-3-yl)-2-(6-(3-fluoropyrrolidin-1-yl)pyridin-3-yl)thiazole-5-carboxamide ³H-Compound 12

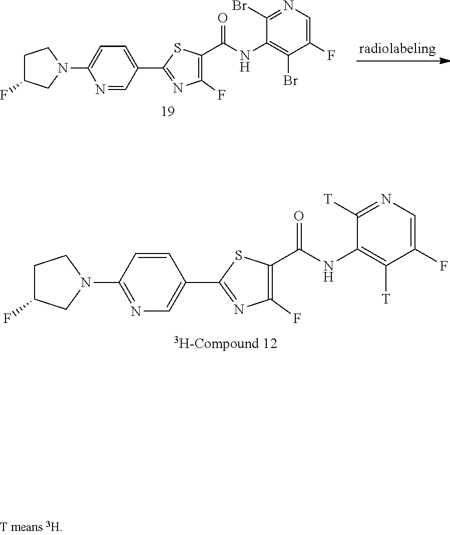

2.48 mg of the dibromo precursor (19), 34 mg of Lindlar catalyst and 50 μl DIPEA were suspended in 0.3 ml DMF. The suspension was degassed three times at the high vacuum manifold and stirred under an atmosphere of tritium gas (5.7 Ci) for 12 h at room temperature. The solvent was removed in vacuo, and labile tritium was exchanged by adding 0.3 ml of methanol, stirring the solution, and removing the solvent again under vacuo. This process was repeated twice. The dried solid was extracted with 10 ml methanol/DCM (1:1) and the suspension was filtered through a 0.2 μm nylon membrane, obtaining a clear solution. For the purification of the compound, the following HPLC conditions were used: Waters Sunfire C18, 10×250 mm; solvents A: water(+0.1% TFA (Sigma-Aldrich T6508); B: acetonitrile+0.1% TFA. The purified product ³H-Compound 12 was obtained (SA 49.23 Ci/mmol, 99% purity).

### Biological Assay Description

### 1. General Methods

Human brain material for these studies was obtained from Prof. William Seeley at the Neurodegenerative Disease Brain Bank UCSF (funding support from NIH grants P01AG019724 and P50AG023501, the Consortium for Frontotemporal Dementia Research, and the Tau Consortium), and also from Prof. Tammaryn Lashley at the Queen Square Brain Bank for Neurological Disorders, UCL. All material has been collected from donors from whom a written informed consent for brain autopsy and the use of the material and clinical information for research purposes has been obtained by the brain bank.

### 1.1 Radioligands

[³H]-Compound 1 which is described above having a specific activity of 49.1 Ci/mmol (1.0 mCi/mL) was used in all competition assays described below, except when [³H]-reference ligand to FTD patient brain-derived TDP-43 aggregates was used (specific activity of 80 Ci/mmol, 1.0 mCi/ml). [³H]-Compound 4 has a specific activity of 44 Ci/mmol (1.0 mCi/mL). [³H]-Compound 8 has a specific activity of 37 Ci/mmol (1.0 mCi/mL). [³H]-Compound 11 has a specific activity of 44 Ci/mmol (1.0 mCi/mL). [³H]-Compound 12 has a specific activity of 29 Ci/mmol (1.0 mCi/mL)

### 1.2. Preparation of Human Frontotemporal Dementia (FTD) Sarkosyl-Insoluble Brain Extracts

Human brain extracts were prepared as described in Laferriere et al., 2019, Nature Neurosc. A sample of brain tissue (frontal or temporal cortex) was homogenized at 1:4 (w/v) ratio in the homogenization-solubilization (HS) buffer at 4° C. using tissue homogenizer (Precellys) with CKmix homogenization tubes. The following sequence was used for homogenization: 3 cycles of 30 seconds at 5000 rpm (with 15 seconds pause between each cycle). Homogenized samples were aliquoted and stored at −80° C. in 1.5 mL low protein binding tubes.

Brain homogenates were thawed on ice and resuspended in HS buffer to obtain a final concentration of 2% sarkosyl, 1 unit/μL Benzonase and 1 mM MgCl₂. The samples were then incubated at 37° C. under constant shaking at 600 rpm on a thermomixer for 45 minutes (min). The supernatants were collected in a new tube (sarkosyl-soluble fraction, S1). The pellet was resuspended in 1000 μL of myelin floatation buffer and centrifuged at 20,000 g for 60 min at 4° C. The supernatant was carefully removed to remove all the floating lipids. This step was repeated if all the lipids could not be removed in a single step. The pellet was subsequently washed with Phosphate-Buffered Saline (PBS) and centrifuged for 30 min at 20,000 g at 4° C. The final pellet was resuspended in 200 μL PBS and stored at −80° C. (sarkosyl-insoluble fraction). The samples were analyzed by immunoblotting in denaturing conditions.

### 2. Biological Assays Description and Corresponding Results

2.1 Kd Determination on Human FTD Sarkosyl Insoluble Brain Extract with Micro-Radiobinding Assay of [³H]-Compound 1, 4, 8, 11, 12

Human FTD sarkosyl-insoluble brain extracts were spotted onto microarray slides. The slides were incubated with [³H]-Compound 1, [³H]-Compound 4, [³H]-Compound 8, [³H]-Compound 11, or [³H]-Compound 12 respectively, in a range of concentrations, varying from 1.93 to 300 nM. After incubation, slides were washed and scanned by a real-time autoradiography system (BeaQuant, ai4R). Quantification of the signal was performed by using the Beamage image analysis software (ai4R). Non-specific signal was determined with an excess of non-radiolabelled compound 1, 4, 8, 11 or 12 (2 μM), respectively, and specific binding was calculated by subtracting the non-specific signal from the total signal.

The Kd (Dissociation constant) and R² (parameter ranging between 0.0 and 1.0 that quantifies the goodness of fit, and the best curve fit obtained with value of 1.0) were obtained by fitting the specific binding data with non-linear regression analysis, using a one-site specific binding model in GraphPad Prism8.

### Results:

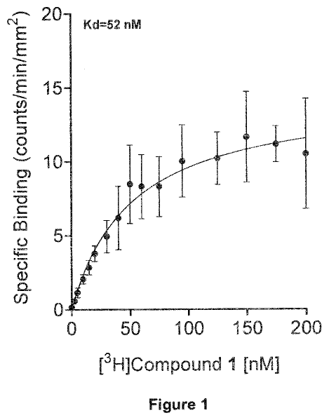

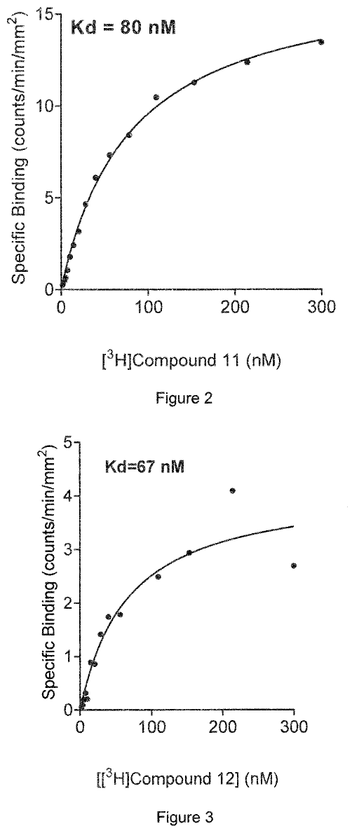

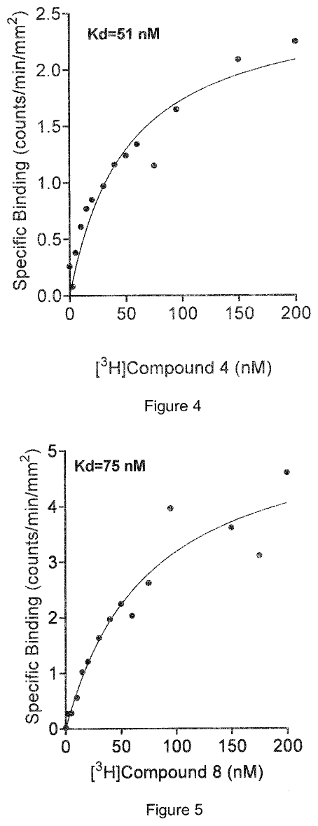

The dissociation constant (Kd) for [³H]-Compounds 1, 4, 8, 11 and 12 were determined on human FTD sarkosyl-insoluble brain extracts in a micro-radiobinding assay. [³H]-Compound 1 had a high specific binding resulting in a high dynamic range and showed a Kd value of 52 nM on human FTD sarkosyl insoluble brain extracts (FIG. 1). Data from two independent experiments resulted in the average Kd of 53±10 nM. [³H]-Compound 11 showed a Kd value of 80 nM (FIG. 2) and [³H]-Compound 12 showed a Kd value of 67 nM (FIG. 3). The dissociation constant for Compounds 4 and 8 were determined on a different batch of FTD sarkosyl-insoluble brain extracts. [³H]-Compound 4 showed a Kd value of 51 nM (FIG. 4). [³H]-Compound 8 showed a Kd value of 75 nM (FIG. 5). These data demonstrate that the compounds of the invention bind with very good affinity to TDP-43 aggregates in human FTD sarkosyl-insoluble brain extracts.

### 2.2. Micro-Radiobinding Competition Assay for the Determination of Binding Affinity

Human FTD sarkosyl-insoluble brain extracts were spotted onto microarray slides. Three batches of extracts were used. The slides were incubated with a tritiated reference ligand at 25 nM or with tritiated Compound 1 ([³H]-Compound 1) at 40 nM or 50 nM depending on the batch used. The example compounds (non-radiolabeled) were used at 1 μM and 125 nM (or 1 μM and 100 nM for compound 15). In some cases, the non-radiolabeled example compounds were further assessed for a range of different concentrations, varying from 0.24 nM to 2 μM for determination of the inhibition constant (Ki). After incubation, slides were washed and scanned by a real-time autoradiography system (BeaQuant, ai4R). Quantification of the signal was performed by using the Beamage image analysis software (ai4R). Non-specific signal was determined with an excess of non-radiolabeled reference compound (2 μM) and specific binding was calculated by subtracting the non-specific signal from the total signal. Competition was calculated as percent, where 0% was defined as the specific binding in the presence of vehicle and 100% as the values obtained in the presence of excess of the non-radiolabeled reference compound. Kᵢ values were calculated in GraphPad Prism8 by applying a nonlinear regression curve fit using a one site, specific binding model. Measurements were performed with at least two technical replicates in the two-concentration competition experiment and with one technical replicate in the experiments including a range of concentrations. For compounds tested in more than one experiment, the mean of the replicates or K; values in independent experiments is reported.

Results: Example compounds 1 to 10 were assessed for their potency to compete with the binding of [³H]-reference ligand to FTD patient brain-derived TDP-43 aggregates. Results of the micro-radiobinding competition assay for the example compounds are shown in Table 2 below as: % competition at 1 μM and 125 nM. Kᵢ values are also shown in Table 2.

Example compounds 11 to 15 were assessed for their potency to compete with the binding of [³H]-Compound 1 on two different batches of brain extracts. Results of the micro-radiobinding competition assay for the example compounds are shown in Table 3 below as: % competition at 1 μM and 125 nM (with Compound 1 at 50 nM) for compounds 11 to 14, or % competition at 1 μM and 100 nM (with Compound 1 at 40 nM) for compound 15 respectively. Kᵢ values are also shown in Table 3.

These data demonstrate that compounds of the invention can bind with good affinity to TDP-43 aggregates.

TABLE 2

| | | Human brain | Human brain | |
|---|---|---|---|---|
| | | derived TDP-43 | derived TDP-43 | |
| | | aggregates | aggregates | Human brain |
| | | Mean % | Mean % | derived TDP-43 |
| | | competition at | competition at | aggregates |
| Compound | Product | 1 μM | 125 nM | Ki [nM] |
| 1 | 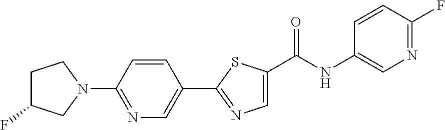 [SMILES: O=C(Nc1ccc(F)nc1)c1cnc(-c2ccc(N3CC[C@@H](F)C3)nc2)s1] | 102 | 63 | 105 |
| | 1 | | | |
| 4 | 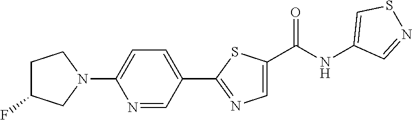 [SMILES: O=C(Nc1cnsc1)c1cnc(-c2ccc(N3CC[C@@H](F)C3)nc2)s1] | 94 | 60 | 116 |
| 5 | 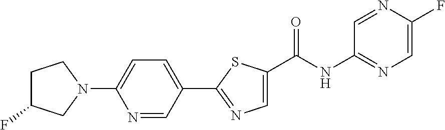 [SMILES: O=C(Nc1cnc(F)cn1)c1cnc(-c2ccc(N3CC[C@@H](F)C3)nc2)s1] | 70 | 53 | 223 |
| 6 | 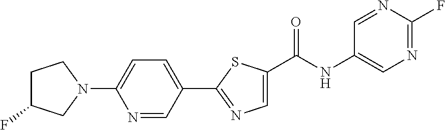 [SMILES: O=C(Nc1cnc(F)nc1)c1cnc(-c2ccc(N3CC[C@@H](F)C3)nc2)s1] | 83 | 46 | 89 |
| 7 | 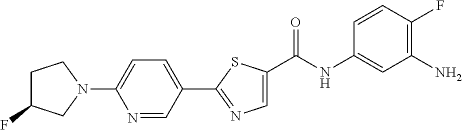 [SMILES: Nc1cc(NC(=O)c2cnc(-c3ccc(N4CC[C@H](F)C4)nc3)s2)ccc1F] | 80 | 52 | 63 |
| 8 | 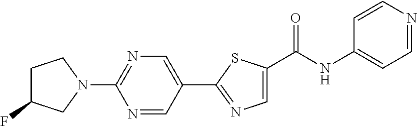 [SMILES: O=C(Nc1ccncc1)c1cnc(-c2cnc(N3CC[C@H](F)C3)nc2)s1] | 78 | 47 | 75 |
| 9 | 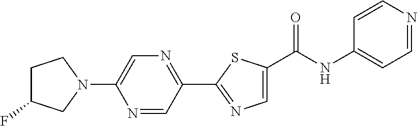 [SMILES: O=C(Nc1ccncc1)c1cnc(-c2cnc(N3CC[C@@H](F)C3)cn2)s1] | 83 | 60 | 82 |
| 10 | 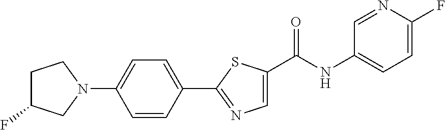 [SMILES: O=C(Nc1ccc(F)nc1)c1cnc(-c2ccc(N3CC[C@@H](F)C3)cc2)s1] | 70 | 54 | 71 |

TABLE 3

| | | Human brain | Human brain | |
|---|---|---|---|---|
| | | derived TDP-43 | derived TDP-43 | |
| | | aggregates | aggregates | Human brain |
| | | Mean % | Mean % | derived TDP-43 |
| | | competition at | competition at | aggregates |
| Example | Product | 1 μM | 125 nM | Ki [nM] |
| 11 | 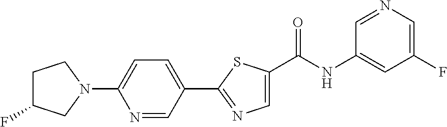 [SMILES: O=C(Nc1cncc(F)c1)c1cnc(-c2ccc(N3CC[C@@H](F)C3)nc2)s1] | 80 | 65 | 50 |
| 12 | 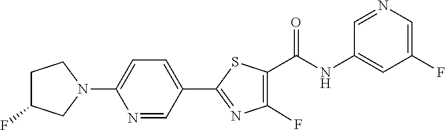 [SMILES: O=C(Nc1cncc(F)c1)c1sc(-c2ccc(N3CC[C@@H](F)C3)nc2)nc1F] | 50 | 43 | 213 |
| 13 | 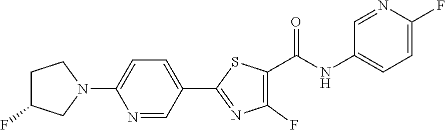 [SMILES: O=C(Nc1ccc(F)nc1)c1sc(-c2ccc(N3CC[C@@H](F)C3)nc2)nc1F] | 75 | 42 | 107 |
| 14 | 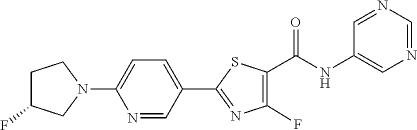 [SMILES: O=C(Nc1cncnc1)c1sc(-c2ccc(N3CC[C@@H](F)C3)nc2)nc1F] | 77 | 38 | 153 |
| 15 | 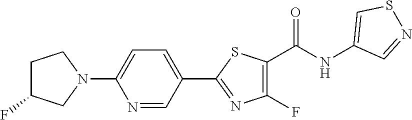 [SMILES: O=C(Nc1cnsc1)c1sc(-c2ccc(N3CC[C@@H](F)C3)nc2)nc1F] | 92 | 47* | 10 |

*Mean % competition at 100 nM

1. A compound having the formula (I) 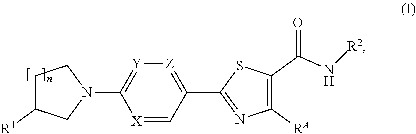
or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N;
R^{A} is H or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.

2. A compound according to claim 1 having the formula (Ia) 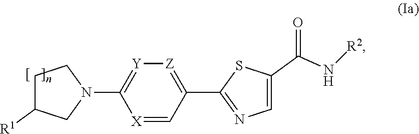
or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.

3. A compound according to claim 1 having the formula (Ib) 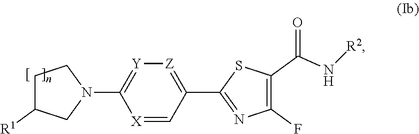
or a detectably labelled compound, stereoisomer, polymorph, racemic mixture, tautomer, pharmaceutically acceptable salt, hydrate, or solvate thereof, or mixtures thereof;
wherein
n is 1 or 2;
R¹ is H or F;
X, Y and Z are independently CH or N; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.

4. The compound according to claim 1, wherein n is 1, and wherein

| | | |
|---|---|---|
| X is N; | Y is CH; | Z is CH; or |
| X is N; | Y is CH; | Z is N; or |
| X is N; | Y is N; | Z is CH; or |
| X is CH; | Y is CH; | Z is CH. |

5. The compound according to claim 1, wherein
R² is 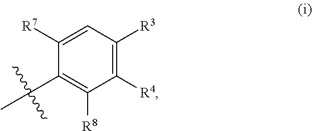
wherein R³ is F, R⁴ is —NH₂, R⁷ and R⁸ are H; or 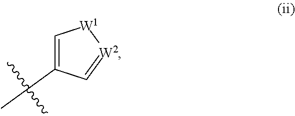
wherein W¹ is N, S or O and W² is N; or 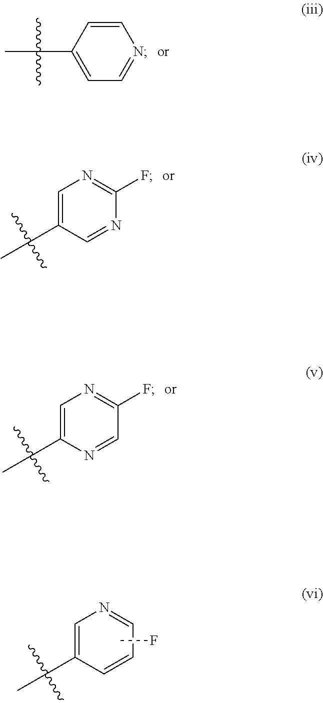
wherein 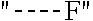
indicates that “F” may be present or absent; or

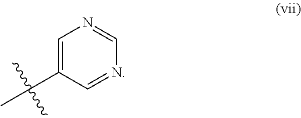 [SMILES: *c1cncnc1]

6. The compound according to claim 1, which is selected from

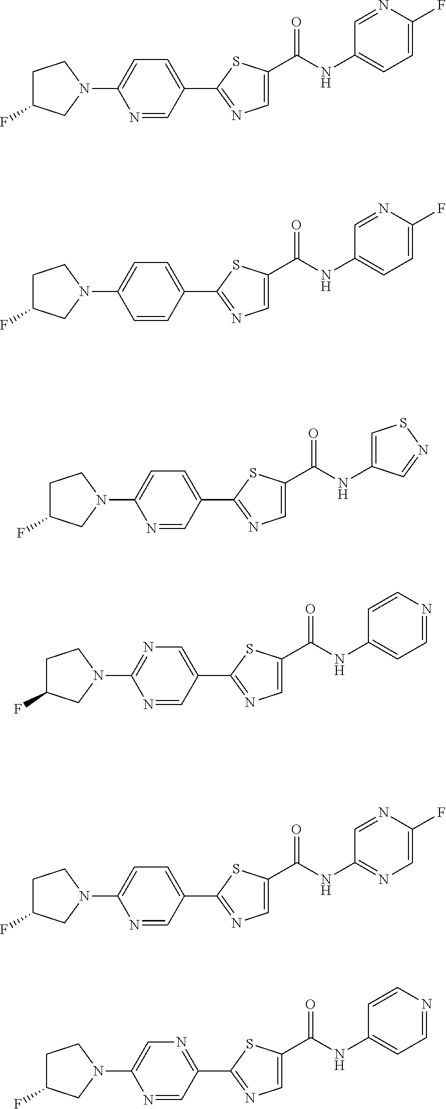 [SMILES: O=C(Nc1ccc(F)nc1)c1cnc(-c2ccc(N3CC[C@@H](F)C3)nc2)s1 ; O=C(Nc1ccc(F)nc1)c1cnc(-c2ccc(N3CC[C@@H](F)C3)cc2)s1 ; O=C(Nc1cnsc1)c1cnc(-c2ccc(N3CC[C@@H](F)C3)nc2)s1 ; O=C(Nc1ccncc1)c1cnc(-c2cnc(N3CC[C@H](F)C3)nc2)s1 ; O=C(Nc1cnc(F)cn1)c1cnc(-c2ccc(N3CC[C@@H](F)C3)nc2)s1 ; O=C(Nc1ccncc1)c1cnc(-c2cnc(N3CC[C@@H](F)C3)cn2)s1]

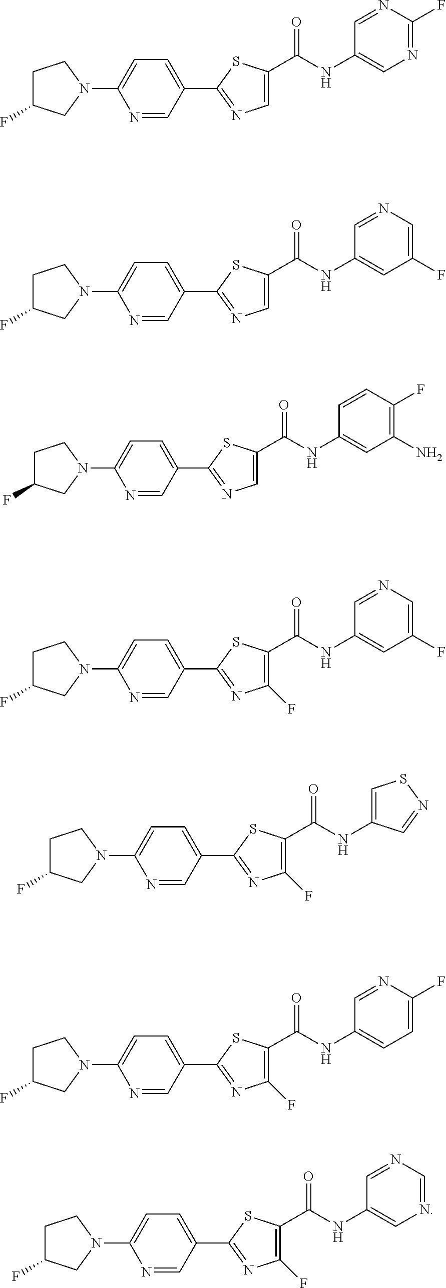

7. The compound according to claim 1, which comprises a detectable label wherein the detectable label is preferably ³H or ¹⁸F.

8. (canceled)

9. The compound according to claim 7, having the formula (I-T) 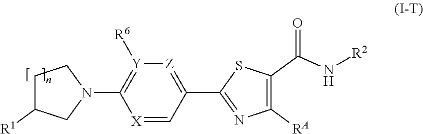
or a stereoisomer, polymorph, racemic mixture, tautomer, pharmaceutically acceptable salt, hydrate, or solvate thereof, or mixtures thereof;
wherein
n is 1 or 2;
R¹ is H or F;
X and Z are independently CH or N;
Y is CR⁶
R⁶ is T or H;
T is ³H;
R^{A} is H or F;
and wherein
R² is 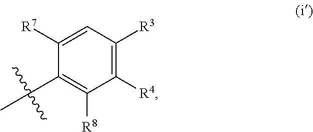
wherein R³ is F, R⁴ is —NH₂, and at least one of R⁷ and R⁸ is T and, if applicable, the other is H; preferably R⁷ and R⁸ are T; or wherein
R² is 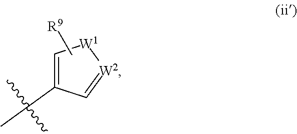
wherein W¹ is N, S or O (preferably S); W² is N, and R⁹ is Tor wherein
R² is 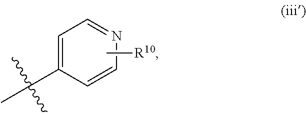
wherein R¹⁰ is T, and R⁶ is T; or wherein
R² is 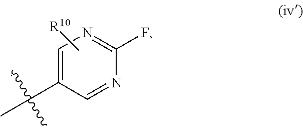
wherein R¹⁰ is T, and R⁶ is T; or wherein
R² is 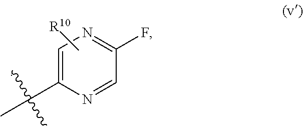
wherein R¹⁰ is T, and R⁶ is T; or wherein
R² is 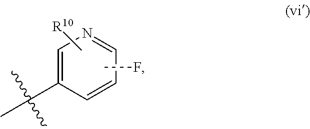
wherein R¹⁰ is T, and R⁶ is T; or wherein
R² is 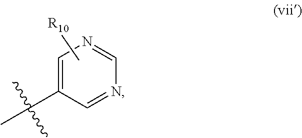
wherein R¹⁰ is T, and R⁶ is T; or wherein
R² is 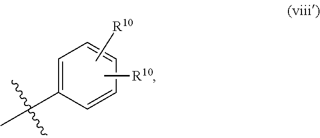
wherein R¹⁰ is T and R⁶ is H; or wherein;
R² is 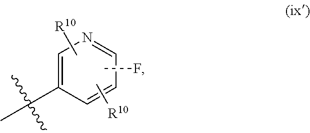
wherein R¹⁰ is T and R⁶ is H; 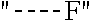
indicates that “F” may be present or absent.

10. The compound according to claim 9, wherein the compound is selected from 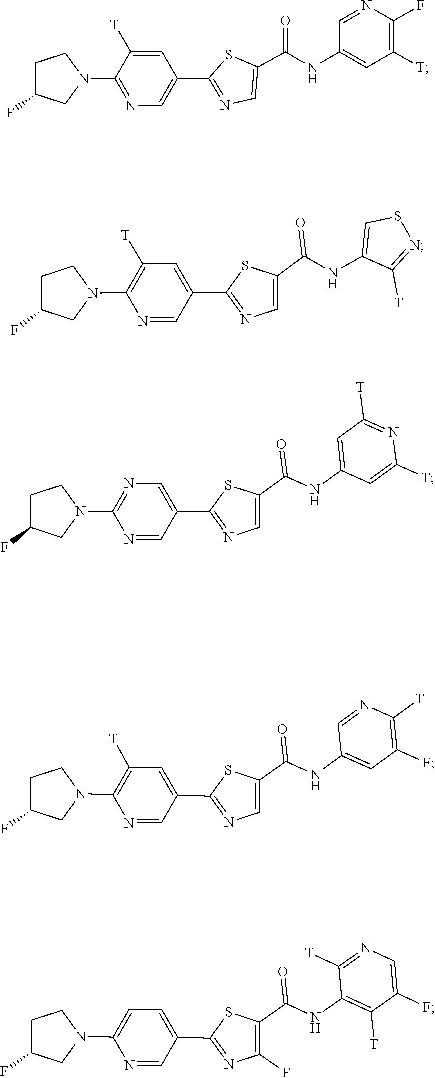
wherein T is ³H.

11. The compound according to claim 7, having the formula (I-F) 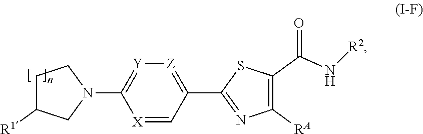
or a stereoisomer, polymorph, racemic mixture, tautomer, pharmaceutically acceptable salt, hydrate, or solvate thereof, or mixtures thereof;
wherein
R^{1′} is ¹⁸F;
n is 1 or 2;
X, Y and Z are independently CH or N;
R^{A} is H or F; and
R² is a 5- or 6-membered carbocyclic or heterocyclic ring which can be optionally substituted with F, NH₂, CN and/or CH₃, wherein the heterocyclic ring contains one or more heteroatoms selected from N, O and/or S.

12. The compound according to claim 11, which is

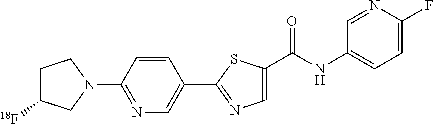 [SMILES: O=C(Nc1ccc(F)nc1)c1cnc(-c2ccc(N3CC[C@@H]([18F])C3)nc2)s1]

13. A diagnostic composition comprising a compound according to claim 7, and optionally at least one physiologically acceptable carrier, diluent, adjuvant and/or excipient.

14. (canceled)

15. A method of imaging of TDP-43 aggregates, wherein a compound according to claim 7 is employed, particularly wherein the imaging is by positron emission tomography.

16. A method of diagnosis of a disease, disorder or abnormality associated with TDP-43 aggregates or of a TDP-43 proteinopathy, or a predisposition thereto, wherein a compound according to claim 7 is employed, particularly wherein the diagnosis is conducted by positron emission tomography.

17. A method which is selected from
(i) a method of diagnosing a disease, disorder or abnormality associated with TDP-43 aggregates or a TDP-43 proteinopathy, or a predisposition thereto, in a subject, the method comprising the steps:
(a) Administering a compound according to claim 7 to the subject;
(b) Allowing the compound to bind to the TDP-43 aggregates;
(c) Detecting the compound bound to the TDP-43 aggregates; and optionally further comprising the steps of:
(d) Generating an image representative of the location and/or amount of the compound bound to the TDP-43 aggregates,
(e) Optionally comparing the generated image with the control image of a healthy control subject, wherein an increase in binding signal indicates that the subject is suffering from or at risk of developing a disease, disorder or abnormality associated with TDP-43 aggregates or a TDP-43 proteinopathy;
(ii) a method of positron emission tomography (PET) imaging of TDP-43 aggregates in a tissue of a subject, the method comprising the steps:
(a) Administering a compound according to claim 7 to the subject;
(b) Allowing the compound to penetrate the tissue of the subject and to bind to the TDP-43 aggregates; and
(c) Detecting the compound bound to the TDP-43 aggregates by collecting a positron emission tomography (PET) image of the tissue of the subject; wherein the tissue is preferably a tissue of the central nervous system (CNS), an eye tissue, or a brain tissue, more preferably wherein the tissue is brain tissue;
(iii) a method for the detection and optional quantification of TDP-43 aggregates in a tissue of a subject, the method comprising the steps:
(a) Bringing a sample or a specific body part or body area suspected to contain TDP-43 aggregates into contact with a compound according to claim 7;
(b) Allowing the compound to bind to the TDP-43 aggregates;
(c) Detecting the compound bound to the TDP-43 aggregates using positron emission tomography; and
(d) Optionally quantifying the amount of the compound bound to the TDP-43 aggregates;
(iv) a method of collecting data for the diagnosis of a disease, disorder or abnormality associated with TDP-43 aggregates or for the diagnosis of a TDP-43 proteinopathy, or a predisposition thereto, the method comprising the steps:
(a) Bringing a sample or a specific body part or body area suspected to contain TDP-43 aggregates into contact with a compound according to claim 7;
(b) Allowing the compound to bind to the TDP-43 aggregates;
(c) Detecting the compound bound to the TDP-43 aggregates; and
(d) Optionally correlating the presence or absence of the compound bound to the TDP-43 aggregates with the presence or absence of TDP-43 aggregates in the sample or specific body part or body area:
(v) a method of collecting data for monitoring the progression of a disease, disorder or abnormality associated with TDP-43 aggregates or for monitoring the progression of a TDP-43 proteinopathy in a patient, the method comprising the steps:
(a) Bringing a sample, a specific body part or body area suspected to contain TDP-43 aggregates into contact with the compound according to claim 7;
(b) Allowing the compound to bind to the TDP-43 aggregates:
(c) Detecting the compound bound to the TDP-43 aggregates;
(d) Optionally correlating the presence or absence of the compound bound to the TDP-43 aggregates with the presence or absence of TDP-43 aggregates in the sample or specific body part or body area; and
(e) Optionally repeating steps (a) to (c) and, if present, optional step (d) at least one time:
(vi) a method of collecting data for predicting responsiveness of a patient suffering from a disease, disorder or abnormality associated with TDP-43 aggregates or suffering from a TDP-43 proteinopathy to a treatment of the disease, disorder or abnormality associated with TDP-43 aggregates with a medicament or to a treatment of the TDP-43 proteinopathy with a medicament, the method comprising the steps:
(a) Bringing a sample, a specific body part or body area suspected to contain TDP-43 aggregates into contact with a compound according to claim 7;
(b) Allowing the compound to bind to the TDP-43 aggregates:
(c) Detecting the compound bound to the TDP-43 aggregates;
(d) Optionally correlating the presence or absence of the compound bound to the TDP-43 aggregates with the presence or absence of TDP-43 aggregates in the sample or specific body part or body area; and
(e) Optionally repeating steps (a) to (c) and, if present, optional step (d) at least one time.

18. (canceled)

19. (canceled)

20. (canceled)

21. (canceled)

22. (canceled)

23. (canceled)

24. (canceled)

25. (canceled)

26. A TDP-43 aggregates' biomarker, a TDP-43 proteinopathy biomarker, a TDP-43 proteinopathy diagnostic agent, or a TDP-43 proteinopathy diagnostic tool comprising a compound according to claim 7.

27. (canceled)

28. An in vitro analytical reference or an in vitro screening tool comprising a compound according to claim 7.

29. The method according to claim 16, wherein the disease, disorder or abnormality associated with TDP-43 aggregates or the TDP-43 proteinopathy is selected from Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

30. The method according to claim 29, wherein the disease, disorder or abnormality associated with TDP-43 aggregates or the TDP-43 proteinopathy is selected from amyotrophic lateral sclerosis (ALS), Alzheimer's disease (AD), Frontotemporal dementia (FTD) including Frontotemporal lobar dementia TDP-43, Frontotemporal lobar degeneration with TDP-43 inclusions (FTLD-TDP), and limbic-predominant age-related TDP-43 encephalopathy (LATE).

31. (canceled)

32. (canceled)

33. (canceled)

34. A compound having the formula (II) 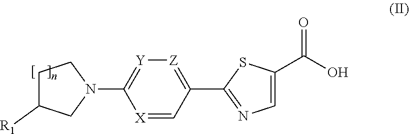
or a stereoisomer, polymorph, racemic mixture, tautomer, pharmaceutically acceptable salt, hydrate, or solvate thereof, or mixtures thereof,
wherein
n is 1 or 2;
X, Y and Z are independently CH or N; and
R¹ is H or F.

35. A compound having the formula (II′) 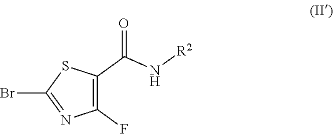
wherein R² is as defined in claim 1.

36. A compound having the formula (III) 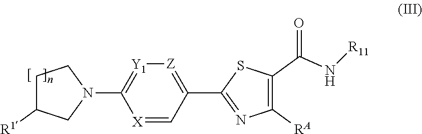
or a stereoisomer, polymorph, racemic mixture, tautomer, pharmaceutically acceptable salt, hydrate, or solvate thereof, or mixtures thereof,
wherein
n is 1 or 2;
R¹ is H or F;
Z is CH or N;
Y¹ is selected from C—Br, C—I, and C—H;
R^{A} is H or F;
R¹¹ is a 5- or 6-membered carbocyclic or heterocyclic ring which can be optionally substituted with Br, I, F, NH₂, CN and/or CH₃, wherein the heterocyclic ring contains one or more heteroatoms selected from N, O and S;
and at least one of Y¹ and/or R¹¹ comprises Br or I.

37. A compound having the formula (IV), 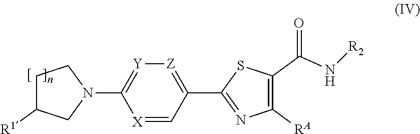
or a stereoisomer, polymorph, racemic mixture, tautomer, pharmaceutically acceptable salt, hydrate, or solvate thereof, or mixtures thereof,
wherein
n is 1 or 2;
X, Y and Z are independently CH or N;
R^{A} is H or F;
R² is 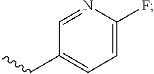
and
R¹² is a leaving group (LG), preferably a mesylate group.

38. A kit for preparing a radiopharmaceutical preparation comprising a compound of formula (III) as defined in claim 36.

39. The method according to claim 17, wherein the disease, disorder or abnormality associated with TDP-43 aggregates or the TDP-43 proteinopathy is selected from Frontotemporal dementia (FTD, such as Sporadic or familial with or without motor-neuron disease (MND), with progranulin (GRN) mutation, with C9orf72 mutations, with TARDBP mutation, with valosine-containing protein (VCP) mutation, linked to chromosome 9p, corticobasal degeneration, frontotemporal lobar degeneration (FTLD) including Frontotemporal lobar dementia TDP-43 or Frontotemporal lobar degeneration with TDP-43 inclusions (FTLD-TDP), Argyrophilic grain disease, Pick's disease, semantic variant primary progressive aphasia (svPPA), behavioural variant FTD (bvFTD), Nonfluent Variant Primary Progressive Aphasia (such as nfvPPA), Amyotrophic lateral sclerosis (ALS, such as Sporadic ALS, with TARDBP mutation, with angiogenin (ANG) mutation), Alexander disease (AxD), limbic-predominant age-related TDP-43 encephalopathy (LATE), Chronic Traumatic Encephalopathy, Perry syndrome, Alzheimer's disease (AD, including sporadic and familial forms of AD), Down syndrome, Familial British dementia, Polyglutamine diseases (Huntington's disease and spinocerebellar ataxia type 3 (SCA3; also known under Machado Joseph Disease)), Hippocampal sclerosis dementia and Myopathies (Sporadic inclusion body myositis, Inclusion body myopathy with a mutation in the valosin-containing protein (VCP); also Paget disease of bone and frontotemporal dementia), Oculo-pharyngeal muscular dystrophy with rimmed vacuoles, Myofibrillar myopathies with mutations in the myotilin (MYOT) gene or mutations in the gene coding for desmin (DES), Traumatic Brain Injury (TBI), Dementia with Lewy Bodies (DLB) and Parkinson's disease (PD), preferably, the disease, the disorder or the abnormality associated with TDP-43 aggregates or the TDP-43 proteinopathy is selected from Frontotemporal dementia (FTD), amyotrophic lateral sclerosis (ALS), Alzheimer's disease (AD), Parkinson's disease (PD), Chronic Traumatic Encephalopathy (CTE), and limbic-predominant age-related TDP-43 encephalopathy (LATE).

40. The method according to claim 39, wherein the disease, disorder or abnormality associated with TDP-43 aggregates or the TDP-43 proteinopathy is selected from amyotrophic lateral sclerosis (ALS), Alzheimer's disease (AD), Frontotemporal dementia (FTD) including Frontotemporal lobar dementia TDP-43, Frontotemporal lobar degeneration with TDP-43 inclusions (FTLD-TDP), and limbic-predominant age-related TDP-43 encephalopathy (LATE).

41. A kit for preparing a radiopharmaceutical preparation comprising a compound of formula (IV) as defined in claim 37.
